(19) United States
(12) Patent Application Publication
(10) Pub. No.: US 2026/0209758 A1
(43) Pub. Date: Jul. 23, 2026

(54) EvolvR Polypeptides and Methods of Use Thereof

(71) Applicant: The Regents of the University of California, Oakland, CA (US)

(72) Inventors: Juan E. Hurtado Koolik, Berkeley, CA (US); David V. Schaffer, Danville, CA (US); John E. Dueber, San Francisco, CA (US); Adam Joseph Schieferecke, Larkspur, CA (US)

(21) Appl. No.: 19/453,890

(22) Filed: Jan. 20, 2026

Related U.S. Application Data
(60) Provisional application No. 63/747,791, filed on Jan. 21, 2025.

(51) Int. Cl. C12N 15/11; C12N 9/12; C12N 9/22; C12N 15/10

(52) U.S. Cl. CPC C12N 15/111; C12N 9/1252; C12N 9/226; C12N 15/1024; C07K 2319/09; C07K 2319/095; C07K 2319/80; C12N 2310/20; C12Y 207/07007

(57) ABSTRACT

The present disclosure provides an EvolvR polypeptides. An EvolvR polypeptide of the present disclosure (i.e., a subject EvolvR polypeptide) comprises: (a) an enzymatically active RNA-guided endonuclease (CRISPR-Cas effector protein such as a Slug-nCas9) that introduces a single-stranded break in a target DNA (i.e., is a nickase); and (b) an error-prone DNA polymerase. In some cases, (a) is fused to (b). In some cases, (a) and (b) are fused to first and second members, respectively, of a dimerization pair, and there (a) and (b) are brought together upon dimerization. In some cases the dimerization is constitutive and in some cases the dimerization is inducible (e.g., using a dimerizing agent).

### CROSS-REFERENCE

This application claims the benefit of U.S. Provisional Patent Application No. 63/747,791 filed Jan. 21, 2025, which application is incorporated herein by reference in its entirety.

### INCORPORATION BY REFERENCE OF SEQUENCE LISTING PROVIDED AS AN XML FILE

A Sequence Listing is provided herewith as a Sequence Listing XML, “BERK-550_SeqList_20260227.xml” created on Feb. 27, 2026 and having a size of 318,998 bytes. The contents of the Sequence Listing XML are incorporated by reference herein in their entirety.

### I. INTRODUCTION

From the late 20th century, researchers have engineered broadly impactful biotechnologies by hypermutating genes of interest and screening the resulting populations of genetic variants to isolate those with enhanced biomolecular function, a method since referred to as directed evolution.

Directed evolution classically begins with the diversification of genes encoding biomolecules of interest in vitro, typically by using error-prone PCR1, DNA shuffling², or saturation mutagenesis³. These genes are stably introduced into cells or viruses either as replicating extrachromosomal elements or genomically-integrated transgenes, such that a given variant's cDNA sequence is retrievable by harvesting the cells' DNA. Upon expression of the genetic library, selective pressures are applied to the host population that either eliminate dysfunctional variants or enhance the persistence or reproduction of functional variants to the degree that they execute the desired function. Such functionalities have included enhanced or novel catalytic activity in enzymes for biomanufacturing⁴; specific and high-affinity binding of proteins with ligands^{5,6}, proteins⁷, or nucleic acids⁸ for research and therapeutic applications; and improved infectivity, tissue-specific tropism, or manufacturing of engineered viral vectors for the delivery of gene therapies to target cells^{9,10,11}. Advantageously, iterative cycles of diversification and selection for desired functions can be executed without prior knowledge of the mechanisms underlying these functionalities. As such, directed evolution is a powerful technique that facilitates the engineering and fine-tuning of incompletely understood biological properties, such as cellular drug resistance or cell tropism of viral vectors.

Despite its historically successful implementation for engineering biotechnology, conventional directed evolution's reliance on discrete steps of library diversification and delivery of the library into a host population limits the diversity of variants that can be tested in each round to the number of host cells that can be transformed, while multiple rounds of directed evolution are labor-intensive as well as limiting for both the number of genes of interest that can be evolved in parallel and the practical duration of directed evolution campaigns. To eliminate diversity bottlenecks related to the transformation of genetic hosts and to improve the scalability of directed evolution campaigns, researchers have developed technologies enabling in vivo continuous evolution of target genes by recruiting error-prone DNA polymerases^{12,13,14,15} or base editors ^{16,17,18,19} to genes of interest directly within host cells, as recently reviewed²⁰. Continuous genetic diversifiers eliminate bottlenecks arising from the transformation of libraries, while continuous and simultaneous diversification and selection allow early “hits” to seamlessly accumulate additional functionally advantageous mutations, allowing unperturbed exploration of arbitrarily distant fitness peaks for as long as the diversifier remains active.

Nevertheless, the directed evolution of new biomolecular properties requires selective pressures that faithfully interrogate these properties. Though activities such as protein-protein binding and enzyme catalysis can depend relatively little on the host cell expressing it, other biological processes of great biotechnological interest can only be interrogated under species and cell type-specific biological contexts. These processes include the signaling kinetics of transmembrane receptors such as GPCRs and synthetic T cell receptors^{21,22}, as well as novel orthogonal receptor-ligand signaling²³. Accordingly, to engineer phenotypes relying on human cell biology via directed evolution, researchers have developed continuous diversifiers that facilitate the targeted hypermutation of genes of interest directly within human cell lines. Existing targeted hypermutators for in vivo continuous evolution in mammalian cells consist of either orthogonal viral polymerases that diversify genes of interest in viral genomes, or CRISPR-guided nucleobase deaminases that chemically mutate C or A nucleotides near gRNA target sites. However, neither of these modalities is suitable for diversifying all four nucleotides at endogenous mammalian genomic loci. Though orthogonal viral error-prone polymerases diversify all nucleotides within genes of interest, the necessary use of viral infection and replication as a means of target gene expression and selection limits the use of techniques such as AdPol and VEGAS to applications in which the gene of interest's functionality can be coupled to viral replication, allows cheater variants to confound directed evolution campaigns²⁴, and relinquishes the capacity to assess functional variants under transcriptionally-normalized and clinically-relevant expression strengths or investigate functional variation arising from perturbation of splicing and regulatory elements. CRISPR-targeted nucleobase deaminases (CRISPR-X and dCas9-AIDx)^{17,18} can efficiently mutate mammalian genomic loci directly, but their reliance on deaminases critically limits the diversity substitutions that can be reliably accessed to transition mutations (C to T, A to G, G to A, T to C), forfeiting access to the vast majority of missense mutations (FIG. 5).

Thus, compositions and methods capable of diversifying all 4 nucleotides within mammalian genomic loci to generate all 12 substitutions would facilitate unprecedented access to previously untapped diversity spaces during in vivo continuous directed evolution campaigns in mammalian cells. Such composition and methods are provided herein.

### II. SUMMARY

The work described in the experimental examples below demonstrate that EvolvR, a targeted mutagenesis method using CRISPR-guided DNA polymerases, can be further engineered to improve diversity generation in mammalian cells. Compared to previous work, compositions and methods described herein can improve mutational efficiencies, achieve a more even distribution of mutational frequencies across editing windows, and overcome limitations associated with delivering and expressing full-length RNA-guided nickase-polymerase complexes to cells.

EvolvR localizes error-prone DNA synthesis to a user-defined locus by generating a priming nick at a target site. Typically, a nickase composed of Cas9 harboring mutation D10A such that it cuts only the gRNA target strand (nCas9) can be fused to an error-prone E. coli Pol I and can be directed by a gRNA to generate a single-stranded break at a target locus. After nCas9 dissociation, Pol I is thought to utilize the nicked strand's exposed 3′ end as a primer for low-fidelity DNA synthesis, displacing the incumbent strand as it generates substitution mutations (FIG. 1A). Substitutions are “locked in” upon evasion of mismatch repair or cell division, generating new library members. As with CRISPR-guided deaminases, EvolvR's capacity to diversify genes directly within the native genome eliminates screening bottlenecks arising from inefficiencies in delivery of pre-generated libraries to host cells. However, unlike CRISPR-guided deaminases, EvolvR leverages error-prone DNA synthesis for mutagenesis and diversifies all four nucleotides.

As such, the inventors have engineered improvements into EvolvR to generate diversity in mammalian cells with high efficiency and large window lengths per gRNA. For example, through experimentation, the inventors surprisingly found that among the many different Cas9 proteins tested, Staphylococcus lugdunensis nickase Cas9 (Slug-nCas9) provided the best mix of desirable properties. For example, to compensate for gRNA-dependent variability in EvolvR's performance, the inventors developed an EvolvR using Slug-nCas9-a high-fidelity PAM-flexible Cas9 ortholog—to drastically increase the number of gRNAs that can be used to functionally target EvolvR for the diversification of a targeted locus. EvolvR was used to identify previously unreported drug-resistant MAP2K1 variants in A375 melanoma cells generated via transversion mutations. The inventors further discovered that EvolvR's mutation window and substitution biases are limited by mismatch tolerance as determined both by the gRNA sequence and a given Cas9 variant's biochemical properties and refined EvolvR by modifying its nickase and gRNA properties to improve mutation outcomes. Moreover, PAM-flexible targeting considerably increases the availability of potential target loci, enabling the diversification of virtually any position within the human genome.

In addition to using knowledge of gRNA mismatch tolerance to improve the mutational efficiency and mutational frequency distribution generated by EvolvR, the inventors have overcome hurdles to delivering and expressing full-length RNA-guided nickase-polymerase fusion proteins. For example, many RNA-guided nickase-polymerase fusion proteins are too large to be delivered by a lentivirus vector containing a marker gene. Even when delivered to target cells by other means (e.g. plasmid transfection), the inventors have discovered that the size of the fusion limits expression levels, which can affect editing efficiency. To overcome this problem, the inventors demonstrate that heterodimerizing nickase and polymerase components of EvolvR (e.g., via fusion of each to a member of a dimerizing pair) can be separately encoded to facilitate improved delivery to target cells.

Therefore, the present disclosure provides new compositions and methods for diversifying all four nucleotides efficiently within native genomic loci in mammalian cells.

Provided are compositions and methods for using such compositions (e.g., methods of modifying a target DNA). For example, provided are compositions that include an EvolvR polypeptide that includes: (a) a Staphylococcus lugdunensis nickase Cas9 (Slug-nCas9) capable of introducing a single-stranded break in a target DNA; and (b) an error-prone DNA polymerase capable of synthesizing a new strand on the target DNA. In some cases, the EvolvR polypeptide is a fusion polypeptide comprising the Slug-nCas9 fused to the DNA polymerase (i.e., the interaction between the Slug-nCas9 and the DNA polymerase is covalent). In some cases, the Slug-nCas9 is fused to a first member of a dimerization pair and the DNA polymerase is fused to a second member of the dimerization pair, such that the first and second members dimerize (in some cases via a dimerizer) to bring together the Slug-nCas9 and the DNA polymerase (i.e., the interaction between the Slug-nCas9 and the DNA polymerase is non-covalent). In some cases, the Slug-nCas9 recognizes an NNGR protospacer adjacent motif (PAM) site. In some cases, the Slug-nCas9 recognizes a more flexible PAM site (e.g., an NNG PAM site). In some cases, the Slug-nCas9 contains or is appended to a nuclear export sequence (NES).

In some cases, the DNA polymerase is an Escherichia coli DNA polymerase I. In some cases, the DNA polymerase comprises an amino acid that is at least 85% identical to the Escherichia coli DNA polymerase I amino acid sequence of SEQ ID NO: 3. In some cases, the DNA polymerase is a DNA polymerase beta, a DNA polymerase iota, a DNA polymerase nu, a DNA polymerase eta, or a DNA polymerase kappa. In some cases, the DNA polymerase includes D424A, I709N, and A759R mutations as numbered relative to SEQ ID NO: 3. In some cases, the DNA polymerase comprises D424A, I709N, A759R, F742Y, and P796H mutations as numbered relative to SEQ ID NO: 3. In some cases, the DNA polymerase does not include an N-terminal flap endonuclease domain (see, e.g., Poll5MΔ (1-325) of SEQ ID NO: 9). In some cases, the DNA polymerase contains or is appended to a nuclear export sequence (NES).

Reagents, compositions, and kits/systems that find use in practicing the subject methods are provided.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A-1E demonstrates that EvolvR facilitates targeted diversification of mammalian genomic loci. FIG. 1A depicts the activity of EvolvR. EvolvR consists of a Cas9 with an inactivated RuvC domain (nCas9) fused to an error-prone DNA polymerase. First, nCas9 generates an RNA-guided single-stranded break in the gRNA target strand. After nCas9 dissociation, the error-prone DNA polymerase performs error-prone DNA synthesis, displacing the incumbent strand and generating substitution errors in the process. Pink lines represent the position of the PAM site. Red crosses represent the presence of a Cas9-generated single-stranded break. FIG. 1B shows a schematic of the BFP-to-GFP assay. The schematic depicts a fluorescent reporter gene for EvolvR mutagenesis using an example gRNA generating a nick in the sense strand 15 bp in the 5′ direction from the codon encoding residue H67. Substitution 199C>T results in missense mutation H67Y and yields expression of GFP instead of BFP. Top to bottom: SEQ ID NOs: 193-197. FIG. 1C provides the experimental setup. HEK293 cells expressing BFP are transfected with a plasmid encoding strong expression of mCherry-tagged EvolvR. Transfection efficiency is quantified the following day to allow normalization of the final frequency of GFP positive cells by the total frequency of cells expressing EvolvR on day 1. On day 6, the cells are harvested and analyzed by flow cytometry to quantify the frequency of GFP positive cells. FIG. 1D demonstrates that both enhanced nickase Cas9 (enCas9) and error-prone Pol I are required for increasing the frequency of H67Y BFP to GFP mutations at the target locus. The percentage of GFP positive cells generated by each construct was measured by flow cytometry and normalized by transfection efficiency. Green dots represent separate biological replicates. “Off target” indicates the expression of a gRNA targeting safe harbor locus AAVS1. All other constructs were coexpressed with a gRNA targeting a nick 15 bp upstream of the codon encoding H67. Double asterisks indicate a p value of less than 0.01, as determined by a two-way ANOVA and Tukey's HSD test. FIG. 1E shows that EvolvR elevates substitution rates throughout the gRNA target sequence and further downstream of the nick as measured by the next-generation sequencing of BFP amplicons. The total frequency of substitution variants was quantified at each position within the BFP reference sequence. Positional coordinates are written with reference to the expected direction of the polymerase, where more positive numbers are further in the 3′ direction with respect to the gRNA target strand. Position 0 corresponds to the position of the nick generated by enCas9. The blue rectangle marks the footprint of the gRNA target sequence. The green vertical line marks the position of the C to T substitution conferring the H67Y missense mutation.

FIG. 2A-2D demonstrates that EvolvR-mediated directed evolution uncovered novel drug-resistant MAP2K1 exon 6 variants. The diagram in FIG. 2A depicts the use of three gRNAs targeting the sense strand in green and the antisense strand in red of exon 6 of MAP2K1. Black markers represent enCas9 cut sites. Top to bottom: SEQ ID NOs: 198-201. The diagram in FIG. 2B depicts the workflow used to evolve new selumetinib-resistant MAP2K1 variants using EvolvR. A375 cells are transiently transfected with plasmids encoding expression of EvolvR and gRNAs targeting MAP2K1. Cells were cultured without selection to allow MAP2K1 variants to evolve before initiating selection with media containing 1 μM selumetinib. Cells were serially passaged until colonies growing rapidly under selective media were visible with the naked eye. gDNA was harvested from the cells and amplicon-sequenced to identify substitution mutations that are enriched in the population. The table in FIG. 2C shows the substitution types and resulting missense mutations of variants enriched under selection of A375 cells by selumetinib after being transfected by EvolvR. All substitution variants enriched cannot be generated by deaminases. Fold-enrichment is quantified by dividing the frequency of substitution variants generated by EvolvR by the frequency of those substitution variants in non-transfected cells. Allele labels indicate that mutations were co-represented and appeared together in all reads, resulting in a single selumetinib-resistant variant. Multiple numbers under “Mut. Fold enrichment over WT” correspond to the variant's enrichment in multiple replicates. Enrichment of variants in different biological replicates are distinguishable by the green, blue, and purple text in the column indicating fold-enrichment. FIG. 2D shows that all variants enriched by at least 227-fold exhibited higher MAP2K1-ERK signaling activity than wildtype MAP2K1 at all concentrations tested. MAP2K1-ERK signaling as measured by SRE-driven expression of a luciferase reporter. Error bars represent 95% confidence intervals.

FIG. 3A-3F demonstrates that enhanced nickase fidelity improves the diversity and frequency of EvolvR substitutions. The schematic in FIG. 3A represents two gRNAs of different lengths targeting the same target sequence within BFP, where one gRNA is 20 nt in length and the other 18 nt in length. gRNA sequences are labelled in red. gBFP15's PAM sequence is labelled in gold. Top to bottom: SEQ ID NOs: 202-205. FIG. 3B provides that truncating gBFP15 enhances the mutation rate of EvolvR when nicking Cas9 is fused to Poll but not when a high-fidelity nickase is fused to Poll. The percentage of GFP positive cells generated by each construct was measured by flow cytometry and normalized by transfection efficiency. Green dots represent separate biological replicates. Black lines represent mean percent GFP positive cells. FIGS. 3C and 3D demonstrate that the use of a truncated gRNA enhances the diversity generated by nCas9-Poll5M relative to when a full-length gRNA is used. The total frequency of substitution variants was quantified at each position within the BFP reference sequence (FIG. 3C). Positional coordinates are written with reference to the expected direction of the polymerase, where more positive numbers are further in the 3′ direction with respect to the gRNA target strand. Position 0 corresponds to the position of the nick generated by nCas9. The blue rectangle marks the footprint of the gRNA target sequence. The number of detectable unique substitution mutations across all four nucleotides increased when EvolvR was coexpressed with a truncated gRNA (tru-gBFP15) (FIG. 3D). Dots represent the mean frequency of individual substitution variants within the gRNA target sequence 18 bp from the PAM site (positions-3 to 15 in c) across two biological replicates. Asterisk represents a p value of less than 0.05 as calculated using a two-tailed student's t test. FIG. 3E shows that the use of a truncated gRNA decreases the total frequency of indels generated by nCas9-Poll5M relative to when a full-length gRNA is used. Double asterisks and single asterisks represents a p value less than 0.01 and 0.05 respectively as calculated by a one-way ANOVA and Tukey's HSD test. The diagram in FIG. 3F depicts proposed mechanism underlying increases in substitution rates at certain positions within the gRNA target sequence when truncated gBFP15 is used instead of full length gBFP15. Substitutions that are tolerable to nCas9 with full-length gRNAs may not be tolerable when complexed with truncated gRNAs, protecting new substitution errors on the target strand from additional cycles of strand displacement.

FIG. 4A-4C demonstrates that a high-fidelity, PAM-flexible nickase targeting the sense strand facilitates consistent mutagenesis of bp within the gRNA target sequence. The schematic in FIG. 4A represents a panel of 24 gRNAs targeting the sense and antisense strands both upstream and downstream of H67Y using NNG PAM sites. gRNA sequences are depicted in purple, while PAM sequences are depicted in gold. Lowercase g's correspond to mismatched 5′ terminal guanines. Labels next to gRNA sequences indicate the position of the cut generated by the gRNA followed by the strand cut, the length of the gRNA, and the complementarity of the 5′ terminal guanine to the target sequence. Lowercase g's indicate that the 5′ terminal guanine is mismatched to the target sequence. Top to bottom: SEQ ID NOs: 206-231. FIG. 4B shows that NNG-Slug-nCas9-Poll5MΔ (SEQ ID NO: 25) reliably mutates positions downstream of nCas9-generated nick overlapping with the gRNA target sequence. The percentage of GFP positive cells generated by each construct was measured by flow cytometry and normalized by transfection efficiency. Dots represent individual biological replicates. Identically colored dots indicate biological replicates of the same gRNA condition. Diamonds represent gRNAs that overlap with nucleotide substitution 199C>T, while circles represent gRNAs not overlapping with nucleotide substitution 199C>T. FIG. 4C provides a Sankey diagram demonstrating that NNG-Slug-nCas9-Poll5MΔ (SEQ ID NO: 25) (NNG-Slug-nCas9-Poll5M is SEQ ID NO: 24) consistently generates GFP positive cells in over half of gRNAs nicking upstream of H67Y on the sense strand, but not in gRNAs targeting the antisense strand. gRNAs targeting the antisense strand or nicking downstream of H67 do not generally generate H67Y missense mutations consistently.

FIG. 5 demonstrates that transition mutations facilitate a minority of missense mutations. This heatmap shows the minimum number of substitutions necessary for converting each of 64 codons to encode any of the other 19 amino acids or stop codons (represented by an asterisk). The left heatmap represents missense mutations accessible with all 12 substitutions. The right heatmap represents missense mutations accessible using only transition mutations.

FIG. 6A-6B provides examples of plasmids for expression of EvolvR in human cells. FIG. 6A provides schematics representing the length and composition of EvolvR variants tested. eSpnCas9 represents SpCas9 with mutations D10A, K848A, K1003A, and R1060A introduced (henceforth referred to as enCas9). Numbers represent length in bp. Poll5MΔ indicates the deletion of the first 325 amino acids from Poll5M. The map in FIG. 6B depicts a plasmid encoding EvolvR for expression in mammalian cells which was constructed as an mCherry-tagged enCas9 flanked by two nuclear localization sequences and fused to Poll5M's Klenow fragment with or without Poll5M's flap endonuclease domain.

FIG. 7A-7C EvolvR generated selumetinib-resistant MAP2K1 substitution variants. FIG. 7A, Brightfield microscope images showing representative A375 cell cultures under selective media 36 days after initiation of selection. FIG. 7B, EvolvR targeted to exon 6 of MAP2K1 generated substitution variants that were enriched compared to untransfected cells by 10 to 50,000-fold after 40 days of selection, while enCas9 alone targeted to exon 6 generated fewer substitution variants enriched by at least 10-fold and EvolvR targeted to a nonexisting BFP gene generated no enriched variants. Triangles, diamonds, and circles correspond to different biological replicates. Green, orange, red, and blue points represent A, T, G, and C variants respectively. Green dashed lines mark the 10-fold enrichment threshold. FIG. 7C, Representative CRISPRESSO allele tables showing enriched alleles containing multiple substitutions in cell populations generated by EvolvR targeted to exon 6 of MAP2K1. Top to bottom: SEQ ID NOs: 232-238.

FIG. 8 Mismatch tolerance bias model of EvolvR mutagenesis. The schematic above depicts a proposed mechanism for explaining variability in EvolvR's performance across different gRNA and nickases. Unedited target sites (1) undergo initial recognition nCas9 (2), enabling the initiation of R loop formation (3). Complete R loop formation facilitates nicking Cas9 to generate a single-stranded break (4). Cas9 dissociates after DNA-DNA hybridization (5), allowing Pol I to initiate strand-displacing DNA synthesis (6). After resection of the 5′ flap by native exonucleases (7), nCas9 has the potential to reassociate, nick, dissociate, and perform additional rounds of strand-displacing synthesis (8,9,10,11,12). At steps in which nCas9 is bound to a nicked substrate (4,10), the replication fork can collide with and displace Cas9 resulting in a single-ended double stranded break (13), initiating homology-directed repair which Pol I may participate in. Alternatively, when a mismatched but ligated target locus exists at the point of DNA replication (7), mutations may be stably installed as substitution variants in one of the two daughter cells (14). Green arrows represent steps expected to synergize with EvolvR-mediated mutagenesis, while red arrows represent steps expected to impede EvolvR-mediated mutagenesis.

FIG. 9A-9B EvolvR mutagenicity at a given position depends on gRNA design and nuclease engineering. EvolvR mutagenicity varies depending on gRNA spacer sequence, gRNA design and nuclease variant fused to Poll5MA. The percentage of GFP positive cells generated by each construct was measured by flow cytometry and normalized by transfection efficiency. Green, yellow, red, and black dots represent that three, two, one, or zero out of three biological replicates contained GFP positive cells, respectively. Black lines represent mean % GFP positive cells. gBFP3 and gBFP15 indicate the use of gRNAs enabling nicking 3 bp and 15 bp upstream of H67. 20G indicates the use 20 nt-long gRNAs with additional 5′ terminal guanines.

FIG. 10A-10D PAM-flexible nickases facilitate targeted mutagenesis of BFP when fused to Poll5MA. FIG. 10A, FIG. 10B, Schematics representing the composition of EvolvR variants tested. Numerical values indicate the length of individual components. eNme2Cas9.NR represents a nickase derived from a PAM flexible variant of Neisseria meningitidis (Nme2) Cas9 containing mutations D16A, K104T, D152A, F260L, A263T, A303S, D451V, E932K, N1031S, R1033G, K1044R, Q1047R, V1056A. SlugCas9 represents a PAM nickase derived from Staphylococcus lugdunensis containing mutation D10A. The diagram depicts a BFP fluorescent reporter gene for EvolvR mutagenesis and a panel of spacer sequences generating nicks in either strand and either direction from the nucleotide C199 encoding the H67Y missense mutation. Light orange and purple lines overlap with unique target sequences in the sense strand of BFP, while dark orange and purple lines overlap with unique target sequences in the antisense strand of BFP. Top to bottom: SEQ ID NOs: 239-240 (FIG. 10A); 241-242 (FIG. 10B). FIG. 10C, FIG. 10D, eNme2-nCas9-Poll5MΔ and Slug-nCas9 facilitate guide-dependent mutagenesis of BFP. The percentage of GFP positive cells generated by each construct was measured by flow cytometry and normalized by transfection efficiency. Dots represent individual biological replicates. Black lines represent mean percent GFP positive cells. gRNA numbers correspond to the spacers labelled in the diagrams in a and b. The absence or presence of a “T” after the gRNA label indicates the use of long or truncated gRNAs. eNme2-nCas9-Poll5MΔ was coexpressed with 23 nt-long and 21 nt-long truncated gRNAs, while Slug-nCas9-Poll5MΔ (SEQ ID NO: 27) (Slug-nCas9-Poll5M is SEQ ID NO: 26) was coexpressed with 20 nt-long and 18 nt-long truncated gRNAs.

FIG. 11A-11C Optimal Slug-nCas9-Poll5MΔ mutagenicity requires at least 20 bp of complementarity with target site. FIG. 11A, Map depicts a plasmid encoding expression of EvolvR comprising an mCherry-tagged SlugCas9 (D10A) fused to Poll5MΔ at its C terminus. FIG. 11B, Diagram depicts a panel of 39 gRNAs targeting 8 different spacer sequences in the BFP gene. Decimal labels correspond to the spacer sequence (first digit) followed by an identifier for a gRNA targeting that spacer sequence (second digit). Numbers to the right of gRNA sequences in purple correspond to the length of complementarity to the target sequence. Blue G's correspond to the G complementary to nucleotide C199, which can undergo mutagenesis to T to generate GFP positive cells. Red lowercase g's represent mismatched terminal guanines. Top to bottom: SEQ ID NOs: 243-284. FIG. 11C, Slug-nCas9-Poll5MΔ facilitates guide-dependent mutagenesis of BFP most consistently when gRNAs share 20 or 21 bp of complementarity with the target strand. The percentage of GFP positive cells generated by each construct was measured by flow cytometry and normalized by transfection efficiency. Dots represent individual biological replicates. gRNA numbers correspond to the spacers labelled in b. bp labels under groups of gRNAs indicate the number of nucleotides within a given gRNA that are complementary to their target strand.

FIG. 12A-12B 5′ terminal mismatches affect EvolvR's substitution rates differently depending on gRNA sequence. FIG. 12A, Diagram depicts a panel of eight gRNAs targeting four distinct spacer sequences in the BFP gene. Decimal labels correspond to the spacer sequence (first digit) followed by an identifier for a gRNA targeting that spacer sequence (second digit). Numbers to the right of gRNA sequences in purple correspond to the length of complementarity to the target sequence. Blue G's correspond to the G complementary to nucleotide C199, which can undergo mutagenesis to T to generate GFP positive cells. Red lowercase letters represent mismatched terminal purines, which are added to test EvolvR's mutagenesis using 21 nt-long gRNAs with and without 5′ terminal mismatches. Top to bottom: SEQ ID NOs: 285-294. FIG. 12B, The effect of 5′ terminal mismatched purines in 21 nt-long gRNAs results in guide-dependent effects on EvolvR mutagenesis. The percentage of GFP positive cells generated by each construct was measured by flow cytometry and normalized by transfection efficiency. Dots represent individual biological replicates. Green, yellow, red, or no dots represent that three, two, one, or zero out of three biological replicates contained GFP positive cells, respectively. Black lines represent mean percent GFP positive cells. gRNA labels correspond to labels in (A).

FIG. 13A-13B depict a system for characterizing diversification of user-defined loci in cytoplasmic DNA using the poxvirus vaccinia as a model. Top to bottom: SEQ ID NOs: 295-298.

FIG. 14A-14C provide data showing that non-fused nickase+polymerase expressed from separate plasmids but subsequently bound using a leucine zipper and directed to the target site with an on-target gRNA confer diversification of a transgene encoded in vaccinia virus. FIG. 14A depicts the percent GFP-positive cells following passage of recombinant vaccinia virus containing a stably incorporated BFP gene in HEK293 cells that were previously transfected with a plasmid encoding 1) BFP-targeted gRNA with a target region corresponding to SEQ ID NO: 141 and NNG-PAM-utilizing nSlug-Cas9 (i.e., nicking Slug-cas9/the “nickase”), and a separate plasmid encoding 2) Poll5M (i.e., the “polymerase”). FIG. 14B depicts representative examples of the raw-data dot plots associated with the quantitative data depicted in FIG. 14A. FIG. 14C depicts a rare variant analysis following next-generation (Illumina amplicon) sequencing of the Empty Vector or nngnSLUGCas9-LZ (Strong)+LZ (Basic)-Poll5M samples from FIGS. 14A and 14B. An increased number of unique rare variants were observed in the nngnSLUGCas9-LZ (Strong)+LZ (Basic)-Poll5M sample relative to the Empty Vector control. These data (FIG. 14A-14C) provide an example that the stronger the affinity of the acidic peptide domain (which in this example is appended C-terminal to the nickase) for the basic peptide domain (which in this example is appended N-terminal to the polymerase), the higher the observed mutation rate. Thus, the mutation rate is tunable, i.e., controllable, by using different dimerization pairs.

FIG. 15A-15C provide data showing that non-fused nickase+polymerase expressed from separate plasmids but subsequently bound using a leucine zipper and directed to the target site with a different on-target gRNA confer diversification of a transgene encoded in vaccinia virus. These data provide another example (using a different gRNA relative to that used in FIG. 14A-14C) that the stronger the affinity of the acidic peptide domain (which in this example is appended C-terminal to the nickase) for the basic peptide domain (which in this example is appended N-terminal to the polymerase), the higher the observed mutation rate. Thus, the mutation rate is tunable, i.e., controllable, by using different dimerization pairs.

FIG. 16A-16D provides example nucleic acid and protein sequences, many of which were used in the working examples. As examples: SEQ ID NO: 55 is NES-LZ (Basic)-Poll5M; SEQ ID NO: 56 is NES-nngnSLUGCas9-LZ; SEQ ID NO: 57 is NES-nngnSLUGCas9-LZ; SEQ ID NO: 58 is NES-nngnSLUGCas9-LZ; SEQ ID NO: 59 is NES_Fused NNG-Slug-nCas9-Poll5M; SEQ ID NO: 140 is U6 Promoter used to Express gRNA; SEQ ID NO: 142 is template binding region (+5′ G Overhang) for gRNA used in FIG. 14A-14B.

### IV. DEFINITIONS

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “naturally-occurring” as used herein as applied to a nucleic acid, a protein, a cell, or an organism, refers to a nucleic acid, cell, protein, or organism that is found in nature.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

“Heterologous,” as used herein, refers to a nucleotide or amino acid sequence that is not found in the native nucleic acid or protein, respectively. For example, relative to a Cas9 polypeptide, a heterologous polypeptide can include an amino acid sequence from a protein other than the Cas9 polypeptide (e.g., a protein tag, a detectable marker protein, a localization signal, a DNA polymerase, etc.). Thus, for example, a polymerase polypeptide is heterologous to a Cas9 polypeptide. Likewise, when a guide RNA is engineered to target a eukaryotic target, the engineered guide sequence can be said to be heterologous to the constant region (scaffold) of the guide RNA. When an exogenous nucleic acid or protein is introduced into a cell, it can be referred to as heterologous to the cell.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, nucleotide sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant nucleotide sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such artificial combination can be carried out to join together nucleic acid segments of desired functions to generate a desired combination of functions.

Similarly, the term “recombinant” polypeptide refers to a polypeptide which is not naturally occurring, e.g., is made by the artificial combination of two otherwise separated segments of amino acid sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change can be achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression (the coding sequence can also be said to be operably linked to the promoter). As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a prokaryotic host cell is a genetically modified prokaryotic host cell (e.g., a bacterium), by virtue of introduction into a suitable prokaryotic host cell of a heterologous nucleic acid, e.g., an exogenous nucleic acid that is foreign to (not normally found in nature in) the prokaryotic host cell, or a recombinant nucleic acid that is not normally found in the prokaryotic host cell; and a eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### V. DETAILED DESCRIPTION

### EvolvR Polypeptides

The present disclosure provides an EvolvR polypeptides. An EvolvR polypeptide of the present disclosure (i.e., a subject EvolvR polypeptide) comprises: (a) an enzymatically active RNA-guided endonuclease (CRISPR-Cas effector protein such as a Slug-nCas9) that introduces a single-stranded break in a target DNA (i.e., is a nickase); and (b) an error-prone DNA polymerase. A subject EvolvR polypeptide can also be referred to herein as a “mutator.” In some cases, (a) is fused to (b). In some cases, (a) and (b) are fused to first and second members, respectively, of a dimerization pair, and there (a) and (b) are brought together upon dimerization. In some cases the dimerization is constitutive and in some cases the dimerization is inducible (e.g., using a dimerizing agent).

In some embodiments, the enzymatically active RNA-guided endonuclease is a Staphylococcus lugdunensis nickase Cas9 (Slug-nCas9). As such, in some cases, an EvolvR polypeptide includes: (a) a Staphylococcus lugdunensis nickase Cas9 (Slug-nCas9) (which is capable of introducing a single-stranded break in a target DNA); and (b) an error-prone DNA polymerase capable of synthesizing a new strand on the target DNA.

In some cases, an EvolvR polypeptide of the present disclosure comprises, in order from N-terminus to C-terminus: a) an enzymatically active RNA-guided endonuclease that introduces a single-stranded break in a target DNA (e.g., a Slug-nCas9); and b) an error-prone DNA polymerase. In some cases, an EvolvR polypeptide of the present disclosure comprises, in order from N-terminus to C-terminus: a) an error-prone DNA polymerase; and b) an enzymatically active RNA-guided endonuclease (e.g., a Slug-nCas9) that introduces a single-stranded break in a target DNA.

In some cases, an EvolvR polypeptide of the present disclosure comprises, in order from N-terminus to C-terminus: a) an enzymatically active RNA-guided endonuclease that introduces a single-stranded break in a target DNA (e.g., a Slug-nCas9); b) a peptide linker; and c) an error-prone DNA polymerase.

In some instances, the EvolvR polypeptide comprises one or more nuclear localization signals (NLSs). For example, in some cases, the EvolvR polypeptide comprises a single NLS at or near (e.g., within 50 amino acids of) the N-terminus of the EvolvR polypeptide. In some cases, the EvolvR polypeptide comprises 2, 3, or 4 NLSs at or near (e.g., within 50 amino acids of) the N-terminus of the EvolvR polypeptide. In some cases, the EvolvR polypeptide comprises a single NLS at or near (e.g., within 50 amino acids of) the C-terminus of the EvolvR polypeptide. In some cases, the EvolvR polypeptide comprises 2, 3, or 4 NLSs at or near (e.g., within 50 amino acids of) the C-terminus of the EvolvR polypeptide. In some cases, the EvolvR polypeptide comprises NLSs at or near (e.g., within 50 amino acids of) the C-terminus and at or near (e.g., within 50 amino acids of) the N-terminus (e.g., a single NLS at each, or more than one, e.g., 2, 3, 4, 5, etc. NLS at each). In some cases, the EvolvR polypeptide does not include an NLS (e.g., in some cases the EvolvR polypeptide instead includes a nuclear export signal (NES).

In some cases, an EvolvR polypeptide of the present disclosure comprises, in order from N-terminus to C-terminus: a) an error-prone DNA polymerase; b) a peptide linker; and c) an enzymatically active RNA-guided endonuclease that introduces a single-stranded break in a target DNA (e.g., a Slug-nCas9). In some instances, the EvolvR polypeptide comprises one or more NLSs. For example, in some cases, the EvolvR polypeptide comprises a single NLS at or near (e.g., within 50 amino acids) the N-terminus of the EvolvR polypeptide. In some cases, the EvolvR polypeptide comprises 2, 3, or 4 NLSs at or near (e.g., within 50 amino acids) the N-terminus of the EvolvR polypeptide. In other instances, in some cases, the EvolvR polypeptide comprises a single NLS at or near (e.g., within 50 amino acids) the C-terminus of the EvolvR polypeptide. In some cases, the EvolvR polypeptide comprises 2, 3, or 4 NLSs at or near (e.g., within 50 amino acids) the C-terminus of the EvolvR polypeptide. In some cases, the EvolvR polypeptide comprises one or more NLSs (e.g., 2, 3, 4, or more) NLSs at or near (e.g., within 50 amino acids) each the C-terminus and the N-terminus.

In some cases, an EvolvR polypeptide of the present disclosure includes a linker polypeptide (sometimes referred to as a linker). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)n, glycine-serine polymers (including, for example, (GS)n, (GSGGS)n (SEQ ID NO:108), and (GGSGGS)n (SEQ ID NO:109), where n is an integer of at least one); glycine-alanine polymers; and alanine-serine polymers. Exemplary linkers can comprise amino acid sequences including, but not limited to, GGSG (SEQ ID NO:111), GGSGG (SEQ ID NO: 112), GSGSG (SEQ ID NO:113), GSGGG (SEQ ID NO:114), GGGSG (SEQ ID NO: 115), GSSSG (SEQ ID NO: 116), and the like. Also suitable is a linker having the sequence (GGGGS)n (SEQ ID NO:110), where n is an integer of from 1 to 10 (e.g., 1, 2, 3, 4, 5, 6, 7, 8, 9, or 10). The ordinarily skilled artisan will recognize that design of a peptide conjugated to any desired element can include linkers that are all or partially flexible, such that the linker can include a flexible linker as well as one or more portions that confer less flexible structure.

An EvolvR polypeptide of the present disclosure can exhibit a high degree of processivity. The processivity of DNA synthesis by a DNA polymerase is defined as the number of nucleotides that a polymerase can incorporate into DNA during a single template-binding event, before dissociating from a DNA template.

In some cases, an EvolvR polypeptide of the present disclosure, when complexed with a guide RNA, exhibits a target mutation rate of from 10⁻⁸ to 10⁻² mutations per nucleotide per genome replication event (e.g., 10⁻² to 10⁻⁷, 10⁻² to 10⁻⁶, 10⁻² to 10⁻⁵, 10 2 to 10⁻⁴, 10⁻² to 10⁻³, 10⁻³ to 10⁻⁸, 10⁻³ to 10⁻⁷, 10⁻³ to 10⁻⁶, 10⁻³ to 10⁻⁵, 10⁻³ to 10⁻⁴, 10⁻⁴ to 10⁻⁸, 10⁻⁴ to 10⁻⁷, 10⁻⁴ to 10⁻⁶, 10⁻⁴ to 10⁻⁵, 10-5 to 10⁻⁸, 10-5 to 10⁻⁷, 10-5 to 10⁻⁶, 10-6 to 10⁻⁸, 10-6 to 10⁻⁷, or 10⁻⁷ to 10⁻⁸). In some cases, an EvolvR polypeptide of the present disclosure, when complexed with a guide RNA, exhibits a target mutation rate of greater than 10⁻⁸ mutations per nucleotide per genome replication event, e.g., greater than 108, greater than 10⁻⁷, greater than 10⁻⁶, greater than 10⁻⁵, greater than 10⁻⁴, greater than 10⁻³, or greater than 10⁻², mutations per nucleotide per genome replication event. In some cases, an EvolvR polypeptide of the present disclosure, when complexed with a guide RNA, exhibits a target mutation rate of from 10⁻⁸ to 10⁻⁷ mutations per nucleotide per genome replication event. In some cases, an EvolvR polypeptide of the present disclosure, when complexed with a guide RNA, exhibits a target mutation rate of from 10⁻⁷ to 10⁻⁶ mutations per nucleotide per genome replication event. In some cases, an EvolvR polypeptide of the present disclosure, when complexed with a guide RNA, exhibits a target mutation rate of from 10⁻⁷ to 10⁻⁵ mutations per nucleotide per genome replication event. In some cases, an EvolvR polypeptide of the present disclosure, when complexed with a guide RNA, exhibits a target mutation rate of from 10⁻⁵ to 10⁻⁴ mutations per nucleotide per genome replication event. In some cases, an EvolvR polypeptide of the present disclosure, when complexed with a guide RNA, exhibits a target mutation rate of from 10⁻⁴ to 10⁻³ mutations per nucleotide per genome replication event. In some cases, an EvolvR polypeptide of the present disclosure, when complexed with a guide RNA, exhibits a target mutation rate of from 10⁻⁴ to 10⁻² mutations per nucleotide per genome replication event. In some cases, an EvolvR polypeptide of the present disclosure, when complexed with a guide RNA, exhibits a target mutation rate of from 10⁻³ to 10⁻² mutations per nucleotide per genome replication event.

In some cases, an EvolvR polypeptide of the present disclosure, when complexed with a guide RNA, exhibits a target mutation rate of 1 mutation per nucleotide per genome replication event.

In some cases, an EvolvR polypeptide of the present disclosure, when complexed with a guide RNA, exhibits a ratio of target mutation rate to global mutation rate of at least 1.5:1, at least 2:1, at least 5:1, at least 10:1, at least 25:1, at least 50:1, at least 10²:1, at least 5×10²:1, at least 10³:1, at least 5×10³:1, at least 10⁴:1, or more than 10⁴:1. In some cases, an EvolvR polypeptide of the present disclosure, when complexed with a guide RNA, exhibits a ratio of target mutation rate to global mutation rate of from about 1.5:1 to 10⁴:1, e.g., from about 1.5:1 to 2:1, from 2:1 to 5:1, from 5:1 to 10:1, from 10:1 to 25:1, from 25:1 to 50:1, from 50:1 to 10²:1, from 10²:1 to 5×10²:1, from 5×10²:1 to 10³:1, from 10³:1 to 5×10³:1, from 5×10³:1 to 10⁴:1, or more than 10⁴:1.

In some cases, an EvolvR polypeptide of the present disclosure, when complexed with a guide RNA, exhibits a target mutation rate that is at least 2-fold higher than the target mutation rate exhibited by the error-prone DNA polymerase present in the EvolvR polypeptide when the error-prone DNA polymerase is not part of the RNA-guided endonuclease present in the EvolvR polypeptide. In some cases, an EvolvR polypeptide of the present disclosure, when complexed with a guide RNA, exhibits a target mutation rate that is at least 2-fold, at least 5-fold, at least 10-fold, at least 50-fold, at least 10²-fold, at least 5×10²-fold, at least 10³-fold, at least 5×10³-fold, or at least 10⁴-fold, higher than the target mutation rate exhibited by the error-prone DNA polymerase present in the EvolvR polypeptide when the error-prone DNA polymerase is not part of the RNA-guided endonuclease present in the EvolvR polypeptide. In some cases, an EvolvR polypeptide of the present disclosure, when complexed with a guide RNA, exhibits a target mutation rate that is more than 10⁴-fold higher than the target mutation rate exhibited by the error-prone DNA polymerase present in the EvolvR polypeptide when the error-prone DNA polymerase is not part of the RNA-guided endonuclease present in the EvolvR polypeptide.

In some cases, an EvolvR polypeptide of the present disclosure, when complexed with a guide RNA, introduces mutations at a distance of from 1 nucleotide to 10⁴ nucleotides from a nick in a target DNA introduced by the RNA-guided endonuclease. For example, in some cases, an EvolvR polypeptide of the present disclosure, when complexed with a guide RNA, introduces mutations at a distance of from 1 nucleotide (nt) to 10 nucleotides (nt), from 1 nt to 100 nt, from 1 nt to 150 nt, from 10 nt to 50 nt, from 50 nt to 100 nt, from 50 nt to 150 nt, from 50 nt to 500 nt, from 100 nt to 500 nt, from 500 nt to 10³ nt, from 10³ nt to 5×10³ nt, or from 5×10³ nt to 10⁴ nt from a nick in a target DNA introduced by the RNA-guided endonuclease. In some cases, an EvolvR polypeptide of the present disclosure, when complexed with a guide RNA, introduces mutations at a distance of from 1 nt to 10 nt from a nick in a target DNA introduced by the RNA-guided endonuclease. In some cases, an EvolvR polypeptide of the present disclosure, when complexed with a guide RNA, introduces mutations at a distance of from 1 nt to 25 nt from a nick in a target DNA introduced by the RNA-guided endonuclease. In some cases, an EvolvR polypeptide of the present disclosure, when complexed with a guide RNA, introduces mutations at a distance of from 10 nt to 25 nt from a nick in a target DNA introduced by the RNA-guided endonuclease. In some cases, an EvolvR polypeptide of the present disclosure, when complexed with a guide RNA, introduces mutations at a distance of from 1 nt to 50 nt from a nick in a target DNA introduced by the RNA-guided endonuclease. In some cases, an EvolvR polypeptide of the present disclosure, when complexed with a guide RNA, introduces mutations at a distance of from 10 nt to 50 nt from a nick in a target DNA introduced by the RNA-guided endonuclease. In some cases, an EvolvR polypeptide of the present disclosure, when complexed with a guide RNA, introduces mutations at a distance of from 25 nt to 50 nt from a nick in a target DNA introduced by the RNA-guided endonuclease. In some cases, an EvolvR polypeptide of the present disclosure, when complexed with a guide RNA, introduces mutations at a distance of from 1 nt to 150 nt from a nick in a target DNA introduced by the RNA-guided endonuclease. In some cases, an EvolvR polypeptide of the present disclosure, when complexed with a guide RNA, introduces mutations at a distance of from 1 nt to 100 nt from a nick in a target DNA introduced by the RNA-guided endonuclease. In some cases, an EvolvR polypeptide of the present disclosure, when complexed with a guide RNA, introduces mutations at a distance of from 10 nt to 100 nt from a nick in a target DNA introduced by the RNA-guided endonuclease. In some cases, an EvolvR polypeptide of the present disclosure, when complexed with a guide RNA, introduces mutations at a distance of from 50 nt to 100 nt from a nick in a target DNA introduced by the RNA-guided endonuclease.

In some cases, the EvolvR polypeptide has a length of no more than about 3000 amino acids. In some cases, the EvolvR polypeptide has a length of from about 1000 amino acids to about 3000 amino acids. In some cases, the EvolvR polypeptide has a length of from about 1000 amino acids to about 1250 amino acids, from about 1250 amino acids to about 1500 amino acids, from about 1500 amino acids to about 1750 amino acids, from about 1750 amino acids to about 2000 amino acids, from about 2000 amino acids to about 2250 amino acids, from about 2250 amino acids to about 2500 amino acids, from about 2500 amino acids to about 2750 amino acids, or from about 2750 amino acids to about 3000 amino acids.

Mutations that can be introduced into a target nucleic acid by a subject EvolvR polypeptide include insertions, deletions, substitutions, and the like.

### Slug-nCas9

As noted above, a subject EvolvR polypeptide includes an enzymatically active RNA-guided endonuclease (CRISPR-Cas effector protein) that introduces a single-stranded break in a target DNA (i.e., is a nickase). In some embodiments, the enzymatically active RNA-guided endonuclease (CRISPR-Cas effector protein) is a Staphylococcus lugdunensis nickase Cas9 (“Slug-nCas9”) (also referred to as “nSlug-Cas9”) [the “n” denotes nickase Cas9].

An example of a Slug-nCas9 is provided as SEQ ID NO: 2. The protein of SEQ ID NO: 2 includes a D10A mutation relative to the wild type Slug-Cas9 (also referred to as SlugCas9), which renders it a nickase, and thus a Slug-nCas9.

SlugCas9 natively recognizes NNGR PAM sites, and so does the Slug-nCas9 of SEQ ID NO: 2. Thus, the Slug-nCas9 of SEQ ID NO: 2 can also be referred to as “NNGR-Slug-nCas9” or “NNGR-nSlug-Cas9.” In some cases, the Slug-nCas9 is a variant that allows for more flexible PAM sequence recognition. For example, the Slug-nCas9 of SEQ ID NO: 1 is a variant (a PAM flexible variant) that recognizes NNG instead of NNGR, and thus the Slug-nCas9 of SEQ ID NO: 1 is sometimes referred to as “NNG-Slug-nCas9” (or “NNG-nSlug-Cas9”).

In some cases, the Slug-nCas9 of a subject EvolvR polypeptide is an NNGR-Slug-nCas9. In some cases, the Slug-nCas9 of a subject EvolvR polypeptide is an NNG-Slug-nCas9.

In some cases, the Slug-nCas9 (e.g., NNG-Slug-nCas9) of a subject EvolvR polypeptide includes an amino acid sequence having 85% or more (e.g., 90% or more, 95% or more, 98% or more, 99% or more, or 100%) amino acid sequence identity to SEQ ID NO: 1. In some cases, the Slug-nCas9 of a subject EvolvR polypeptide includes an amino acid sequence having 95% or more (e.g., 98% or more, 99% or more, or 100%) amino acid sequence identity to SEQ ID NO: 1. In some cases, the Slug-nCas9 of a subject EvolvR polypeptide includes the amino acid sequence of SEQ ID NO: 1.

In some cases, the Slug-nCas9 (e.g., NNGR-Slug-nCas9) of a subject EvolvR polypeptide includes an amino acid sequence having 85% or more (e.g., 90% or more, 95% or more, 98% or more, 99% or more, or 100%) amino acid sequence identity to SEQ ID NO: 2. In some cases, the Slug-nCas9 of a subject EvolvR polypeptide includes an amino acid sequence having 95% or more (e.g., 98% or more, 99% or more, or 100%) amino acid sequence identity to SEQ ID NO: 2. In some cases, the Slug-nCas9 of a subject EvolvR polypeptide includes the amino acid sequence of SEQ ID NO: 2.

### CRISPR-Cas Effector Polypeptides

In some embodiments, e.g., when the EvolvR polypeptide is composed of two different proteins (e.g., using a dimerizing pair), the CRISPR-Cas effector polypeptide is a Slug-nCas9. However, in other embodiments, e.g., when the EvolvR polypeptide is composed of two different proteins (e.g., using a dimerizing pair), the CRISPR-Cas effector polypeptide is not a Slug-nCas9.

Examples of CRISPR-Cas effector polypeptides that can be used instead of a Slug-nCas9 include, but are not necessarily limited to: Type II CRISPR-Cas effector polypeptides, Type III CRISPR Cas effector polypeptides, Type V CRISPR Cas effector polypeptides, and Type VI CRISPR-Cas effector polypeptides.

In some cases, the CRISPR-Cas effector polypeptide is a type II CRISPR-Cas effector polypeptide. In some cases, the type II CRISPR-Cas effector polypeptide is a Cas9 polypeptide, e.g., Staphylococcus aureus Cas9, Streptococcus pyogenes Cas9 (SpCas9), etc. In some cases, the CRISPR-Cas effector polypeptide is a variant of a wild-type SpCas9 and comprises one or more of the following substitutions: A61R, L1111R, A1322R, D1135L, S1136W, G1218K, E1219Q, N1317R, R1333P, R1335A, and T1337R. In some cases, the CRISPR-Cas effector polypeptide is an SpG polypeptide or a SpRY polypeptide; see, e.g., Walton et al. (2020) Science 368:290, and WO 2019/051097. SpRY is capable of targeting almost all protospacer-adjacent motifs (PAMs) (NRN>NYN). In some cases, the CRISPR-Cas effector polypeptide is a miniaturized nSpRY-Cas9 for which amino acids have been deleted. For example, a suitable CRISPR-Cas effector polypeptide is an SpCas9 polypeptide includes D1135V, R1135Q, and T1137R substitutions, relative to wild-type SpCas9. As another example, a suitable CRISPR-Cas effector polypeptide is an SpCas9 polypeptide includes D1135V, R1335Q, T1337R, and G1218R substitutions, relative to wild-type SpCas9. As another example, a suitable CRISPR-Cas effector polypeptide is an SpCas9 polypeptide includes D1135L, S1136W, G1218K, E1219Q, R1335A, and T1337R substitutions, relative to wild-type SpCas9. As another example, a suitable CRISPR-Cas effector polypeptide is an SpCas9 polypeptide includes L1111R, A1322R, D1135L, S1136W, G1218K, E1219Q, R1335A, and T1337R substitutions, relative to wild-type SpCas9. As another example, a suitable CRISPR-Cas effector polypeptide is an SpCas9 polypeptide includes A61R, L1111R, A1322R, D1135L, S1136W, G1218K, E1219Q, N1317R, R1333P, R1335A, and T1337R substitutions, relative to wild-type SpCas9. The amino acid sequence of a wild-type SpCas9 polypeptide is provided as SEQ ID NO: 31. As another example, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence of any one of SEQ ID NOs: 31-32, and 43-46.

In some cases, the CRISPR-Cas effector polypeptide is a type V CRISPR-Cas effector polypeptide, e.g., a Cas12a, a Cas12b, a Cas12c, a Cas12d, or a Cas12e polypeptide (see, e.g., SEQ ID NOs: 33-35, and 41-42 for examples of Cas12a; SEQ ID NO: 36 for an example of Cas12b). In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide (see, e.g., SEQ ID NOs: 37 and 39-40 for examples of Cas13 sequences). In some cases, the CRISPR-Cas effector polypeptide is a Cas14 polypeptide. In some cases, the CRISPR-Cas effector polypeptide is a Cas14a polypeptide, a Cas 14b polypeptide, or a Cas14c polypeptide. Cas7-11 is an example of a Type III-E CRISPR Cas effector (see, e.g., SEQ ID NO: 38).

For example, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence of any one of SEQ ID NOs: 31-46. In some cases, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence of any one of SEQ ID NOs: 43-46.

In some cases, a CRISPR-Cas effector polypeptide suitable for use in a method, a system, or a composition of the present disclosure is a nickase CRISPR-Cas effector polypeptide, i.e., a CRISPR-Cas effector polypeptide that, when complexed with a guide RNA, binds to a target nucleic acid and cleaves only one strand of the target nucleic acid. For example, in some cases, a CRISPR-Cas effector polypeptide is a SpyCas9 polypeptide comprising a D10A substitution. In some cases, a CRISPR-Cas effector polypeptide is a Slug-nCas9.

As noted above, a DNA-or protein-modifying enzyme-containing EvolvR polypeptide of the present disclosure (e.g., a deaminase-containing base editor of the present disclosure) comprises an RNA-guided enzyme that exhibits nickase activity. Suitable nickases are described elsewhere herein.

In some cases, a suitable RNA-guided enzyme that exhibits nickase activity comprises an amino acid sequence having at least 80%, at least 85%, at least 90%, at least 95%, at least 98%, at least 99%, or 100%, amino acid sequence identity to the following “nicking high fidelity” Cas9 amino acid sequence:

(SEQ ID NO: 117)
DKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTAFDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGALSRKLINGIRDKQSGKTILDFLKSDG
FANRNFMALIHDDSLTFKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYW
RQLLNAKLITQRKFDNLTKAERGGLSELDKAGFIKRQLVETRAITKHVA
QILDSRMNTKYDENDKLIREVKVITLKSKLVSDFRKDFQFYKVREINNY
HHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRD
FATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKN
PIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAF
KYFDTTIDRKRYTSTKEVLDATLIHQSITGLYETRIDLSQLGGD.

In some cases, a suitable RNA-guided enzyme that exhibits nickase activity comprises an amino acid sequence having at least 80%, at least 85%, at least 90%, at least 95%, at least 98%, at least 99%, or 100%, amino acid sequence identity to the following “nicking enhanced” Cas9 amino acid sequence:

(SEQ ID NO: 118)
DKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLADDSIDNKVLTRSDKNRGKSDNVPSEEVVKKMKNYW
RQLLNAKLITQRKFDNLTKAERGGLSELDKAGFIKRQLVETRQITKHVA
QILDSRMNTKYDENDKLIREVKVITLKSKLVSDFRKDFQFYKVREINNY
HHAHDAYLNAVVGTALIKKYPALESEFVYGDYKVYDVRKMIAKSEQEIG
KATAKYFFYSNIMNFFKTEITLANGEIRKAPLIETNGETGEIVWDKGRD
FATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKN
PIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAF
KYFDTTIDRKRYTSTKEVLDATLIHQSITGLYETRIDLSQLGGD.

In some cases, a suitable RNA-guided enzyme that exhibits nickase activity comprises an amino acid sequence having at least 80%, at least 85%, at least 90%, at least 95%, at least 98%, at least 99%, or 100%, amino acid sequence identity to the following “nicking” Cas9 amino acid sequence:

(SEQ ID NO: 119)
DKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVA
QILDSRMNTKYDENDKLIREVKVITLKSKLVSDFRKDFQFYKVREINNY
HHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRD
FATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKN
PIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAF
KYFDTTIDRKRYTSTKEVLDATLIHQSITGLYETRIDLSQLGGD.

In some cases, in addition to being a nickase, a CRISPR-Cas effector protein (e.g., a Cas9 protein) is a variant. In some cases, such a variant is a high fidelity (HF) protein such as a HF Cas9 protein (also referred to as SpCas9-HF1 or HF1- and also-HF2, -HF3, -HF4) (e.g., see Kleinstiver et al. (2016) Nature 529:490). For example, amino acids N497, R661, Q695, and Q926 can be substituted, e.g., with alanine. In some cases, a suitable parent Cas9 protein exhibits altered PAM specificity, e.g., in some cases the Cas9 is a VRVRFRR Cas9 (which recognizes an NG PAM instead of NGG) see, e.g., Nishimasu et al., Science. 2018 Sep. 21;361 (6408): 1259-1262; and Kleinstiver et al. (2015) Nature 523:481. Additional examples of Cas9 variants that can be used, include, but are not limited to: HiFiCas9 (e.g., R691A), eSpCas9 (e.g., K810A, K1003A, R1060A), eSpCas9 (e.g., D1135E), HypaCas9 (e.g., N692A, M694A, Q695A, H698A), xCas9 (e.g., E108G, S217A, A262T, S4091, E480K, E543D, M6941, E1219V), Sniper-Cas9 (e.g., F539S, M763I, K890N), evoCas9 (e.g., M495V, Y515N, K526E, R661Q), SpartaCas (e.g., D23A, T67L, Y128V, D1251G), LZ3Cas9 (e.g., N690C, T7691, G915M, N980K), miCas9 (e.g., SV40 NLS linker fused with brex27 motif), SuperFi-Cas9 (e.g., Y1010D, Y1013D, Y1016D, V1018D, R1019D, Q1027D, K1031D) (see, e.g., Allemailem et al, Int J Mol Sci. 2023 Apr. 11; 24 (8): 7052).

In some cases, the Cas9 is an iGeoCas9 (see, e.g., international patent publication WO2024112479, which is incorporated herein by reference for such disclosure). For example, in some cases, the CRISPR-Cas effector protein comprises an amino acid sequence that is 80% or more (e.g., 85% or more, 90% or more, 92% or more, 95% or more, 97% or more, 98% or more, 99% or more) identical to iGeoCas9, which is the same as the wild type GeoCas9, but with the following mutations: E149G, T1821, N206D, P466Q, Q817R, E843K, E884G, and K908R (see, e.g., WO2024112479 as well as Chen et al., bioRxiv. Preprint. 2023 Nov. 15: doi: 10.1101/2023.11.15.566339). In some cases, the CRISPR-Cas effector protein comprises an amino acid sequence that is 92% or more (e.g., 95% or more, 97% or more, 98% or more, 99% or more) identical to iGeoCas9.

For additional information related to programmable gene editing tools (e.g., CRISPR-Cas RNA-guided proteins such as Cas9, Cas12a, Cas13, etc., Zinc finger proteins such as Zinc finger nucleases, TALE proteins such as TALENs, CRISPR-Cas guide RNAs, PAMs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163 (3): 759-71; Makarova et al, Nat Rev Microbiol. 2015 November; 13 (11): 722-36; Shmakov et al., Mol Cell. 2015 Nov. 5;60 (3): 385-97; Jinek et al., Science. 2012 Aug. 17;337 (6096): 816-21; Chylinski et al., RNA Biol. 2013 May; 10 (5): 726-37; Ma et al., Biomed Res Int. 2013; 2013:270805; Hou et al., Proc Natl Acad Sci USA. 2013 Sep. 24; 110 (39): 15644-9; Jinek et al., Elife. 2013; 2: e00471; Pattanayak et al., Nat Biotechnol. 2013 September; 31 (9): 839-43; Qi et al, Cell. 2013 Feb. 28; 152 (5): 1173-83; Wang et al., Cell. 2013 May 9; 153 (4): 910-8; Auer et. al., Genome Res. 2013 Oct. 31; Chen et. al., Nucleic Acids Res. 2013 Nov. 1;41 (20): e19; Cheng et. al., Cell Res. 2013 October; 23 (10): 1163-71; Cho et. al., Genetics. 2013 November; 195 (3): 1177-80; DiCarlo et al., Nucleic Acids Res. 2013 April; 41 (7): 4336-43; Dickinson et. al., Nat Methods. 2013 October; 10 (10): 1028-34; Ebina et. al., Sci Rep. 2013; 3:2510; Fujii et. al, Nucleic Acids Res. 2013 Nov. 1;41 (20): e187; Hu et. al., Cell Res. 2013 November; 23 (11): 1322-5; Jiang et. al., Nucleic Acids Res. 2013 Nov. 1;41 (20): e188; Larson et. al., Nat Protoc. 2013 November; 8 (11): 2180-96; Mali et. at., Nat Methods. 2013 October; 10 (10): 957-63; Nakayama et. al., Genesis. 2013 December; 51 (12): 835-43; Ran et. al., Nat Protoc. 2013 November; 8 (11): 2281-308; Ran et. al., Cell. 2013 Sep. 12; 154 (6): 1380-9; Upadhyay et. al., G3 (Bethesda). 2013 Dec. 9;3 (12): 2233-8; Walsh et. al., Proc Natl Acad Sci USA. 2013 Sep. 24; 110 (39): 15514-5; Xie et. al., Mol Plant. 2013 Oct. 9; Yang et. al., Cell. 2013 Sep. 12; 154 (6): 1370-9; Briner et al., Mol Cell. 2014 Oct. 23;56 (2): 333-9; Burstein et al., Nature. 2016 Dec. 22-Epub ahead of print; Gao et al., Nat Biotechnol. 2016 Jul 34 (7): 768-73; Shmakov et al., Nat Rev Microbiol. 2017 March; 15 (3): 169-182; Makarova et al., Nat Rev Microbiol. 2020 February; 18 (2): 67-83; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

### Guide Nucleic Acids

A nucleic acid molecule (e.g., a crRNA or a hybridized crRNA/tracrRNA) that binds to a CRISPR-Cas effector protein (e.g., a Cas9 such as Slug-nCas9, a Cas12 such as Cas12a, a Cas13, etc.), forming a ribonucleoprotein complex (RNP), and targets the complex to a specific target sequence within a target nucleic acid (e.g., target DNA or target RNA) is referred to herein as a “guide RNA” or “gRNA.” It is to be understood that in some cases, a hybrid DNA/RNA can be made such that a guide RNA includes DNA bases in addition to RNA bases—but the term “guide RNA” is still used herein to encompass such hybrid molecules.

A guide RNA provides target specificity to the complex (the RNP complex) by including a targeting segment, which includes a “guide sequence” (also referred to as a “targeting sequence” or a “spacer”), which is a nucleotide sequence that is complementary to (and hybridizes to) a target sequence of a target nucleic acid, e.g., a target DNA, (and thereby can be said to “target” a specific sequence or “target” a specific gene). The guide sequence can be changed each time a new target sequence is selected. In some cases, e.g., in some cases when the CRISPR-Cas effector protein is a Cas9, e.g., a Slug-nCas9, the guide sequence is 17-25 nucleotides (nt) long (e.g., 17-24, 17-23, 17-22, 17-21, 17-20, 17-19, 17-18, 18-25, 18-24, 18-23, 18-22, 18-21, 18-20, 18-19, 19-25, 19-24, 19-23, 19-22, 19-21, 19-20, 20-25, 20-24, 20-23, 20-22, or 20-21 nt). In some cases, the guide sequence is 18-22 nt long (e.g., 18-21, 18-20, 18-19, 19-22, 19-21, 19-20, 20-22, or 20-21 nt). In some cases, the guide sequence is 20nt long. In some cases, the target sequence is 17-25 nucleotides (nt) long (e.g., 17-24, 17-23, 17-22, 17-21, 17-20, 17-19, 17-18, 18-25, 18-24, 18-23, 18-22, 18-21, 18-20, 18-19, 19-25, 19-24, 19-23, 19-22, 19-21, 19-20, 20-25, 20-24, 20-23, 20-22, or 20-21 nt). In some cases, the target sequence is 18-22 nt long (e.g., 18-21, 18-20, 18-19, 19-22, 19-21, 19-20, 20-22, or 20-21 nt). In some cases, the target sequence is 20nt long.

A guide RNA also includes a portion that interacts with (binds to) the CRISPR-Cas effector protein. Because this region does not need to change each time a new target sequence is selected, this region is referred to as a “constant region” or “scaffold” (or “handle” or “repeat” or “protein-binding segment”). As would be known to one of ordinary skill in the art, depending on which type of CRISPR-Cas effector protein/system is being used, in some cases, the constant region is 5′ of the guide sequence (i.e., the guide sequence is 3′ of the constant region) (e.g., in cases where the PAM is located upstream of the target sequence), and in other cases (e.g., when a Cas9 is used), the constant region is 3′ of the guide sequence (i.e., the guide sequence is 5′ of the constant region) (e.g., in cases where the PAM is located downstream of the target sequence).

A guide RNA can be referred to by the protein to which it corresponds. For example, when a CRISPR-Cas effector protein is a Cas9 protein, the corresponding guide RNA can be referred to as a “Cas9 guide RNA.” Likewise, when a CRISPR-Cas effector protein is a Cas12a protein, the corresponding guide RNA can be referred to as a “Cas12a guide RNA,” and when a CRISPR-Cas effector protein is a Cas13 protein, the corresponding guide RNA can be referred to as a “Cas13 guide RNA.”

As will be known to one of ordinary skill in the art, in some embodiments, a guide RNA includes two separate nucleic acid molecules: an “activator” and a “targeter” (or tracrRNA and crRNA) and can be referred to as a “dual guide RNA”, a “double-molecule guide RNA”, a “two-molecule guide RNA”, or a “dgRNA.” In some embodiments, the guide RNA is one molecule (e.g., for some class 2 CRISPR-Cas proteins, the corresponding natural guide RNA is a single molecule; and in some cases, an activator and targeter (crRNA and tracrRNA) can be covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, a guide RNA includes a 5′ terminal purine. In some cases, the 5′ terminal purine is a guanine.

A gRNA can be selected using software. As a non-limiting example, considerations for selecting a gRNA can include, e.g., the PAM sequence for the CRISPR-Cas effector protein to be used, and strategies for minimizing off-target modifications. Tools, such as NUPACK and the CRISPR Design Tool, can provide sequences for preparing the gRNA, for assessing target modification efficiency, and/or assessing cleavage at off-target sites.

As would be understood to one of ordinary skill in the art, the guide RNA can be introduced into a cell as an RNA (or as a DNA/RNA hybrid), or can be introduced as a nucleic acid encoding the RNA (e.g., a DNA such as an expression vector such as a viral, plasmid, or minicircle DNA), in which case the cell transcribes the RNA from the introduced DNA. In some cases, the nucleotide sequence encoding the guide RNA is operably linked to a promoter (e.g., a Pol III promoter such as U6 or H1). In some cases, one or more guide RNAs (e.g., 1, 2, 3, 4, 5, 6, 1-10, 1-8, 1-6, 1-5, 1-4, 1-3, 2-10, 2-8, 2-6, 2-5, 2-4, 3-10, 3-8, 3-6, 3-5, two or more, three or more, four or more, or five or more) (or nucleotide sequences that encode said guide RNAs) can be introduced into the same cell (e.g., to target different sequences of the same target nucleic, to target different target nucleic acids, etc.).

Scaffold sequences for various CRISPR-Cas guide RNAs are known in the art. For example, in some cases, the portion of the targeter-RNA (e.g., crRNA) that contributes to the scaffold (i.e., is 3′ of the guide sequence) (e.g., when using an S. pyogenes Cas9 protein) includes: GUUUUAGAGCUAUGCUGUUUUG (SEQ ID NO: 120). In some cases, it includes: GUUUUAGAGCUA (SEQ ID NO: 121). in some cases, the activator-RNA (e.g., tracrRNA) (e.g., when using an S. pyogenes Cas9 protein) includes:

(SEQ ID NO: 122)
AAACAGCAUAGCAAGUUAAAAUAAGGCUAGUCCGUUAUCAACUUGAAAA
AGUGGCACCGAGUCGGUGCUU.

Examples of scaffold sequences include, but are not limited to:
GUUUUAGAGCUAGAAAUAGCAAGUUAAAAUAAGGCUAGUCCG (SEQ ID NO: 123) (e.g., when using an S. pyogenes Cas9 protein);
GUUUUAGAGCUAGAAAUAGCAAGUUAAAAUAAGGCUAGUCCGUUAUCAACUUG AAAAAGUGGCACCGAGUCGGUGCUU (SEQ ID NO: 124) (e.g., when using an S. pyogenes Cas9 protein); and
GUUUCAGUACUCUGGAAACAGAAUCUACUGAAACAAGACAAUAUGUCGUGUUU AUCCCAUCAAUUUAUUGGUGGGAUUUUU (SEQ ID NO: 139) (e.g., when using a Slug-Cas9 such as a Slug-nCas9 protein such as NNG-Slug-nCas9).

Mutations/variants of the above sequences can also be used and many suitable examples will be known to one of ordinary skill in the art.

Examples of crRNA repeat sequences (also known as the scaffold) for Cas12a proteins include:

```
LbCas12a crRNA:
  (SEQ ID NO: 125)
AAUUUCUACUAAGUGUAGAU-[spacer] 3′
AsCas12a crRNA:
  (SEQ ID NO: 126)
AAUUUCUACUCUUGUAGAU-[spacer] 3′
FnCas12a crRNA:
  (SEQ ID NO: 127)
AAUUUCUACUGUUGUAGAU-[spacer] 3′
PmCas12a crRNA:
  (SEQ ID NO: 128)
AAUUUCUACUAUUGUAGAU-[spacer] 3′
MbCas 12a/Mb2Cas12a/Mb3Cas 12a crRNA:
  (SEQ ID NO: 129)
AAUUUCUACUGUUUGUAGAU-[spacer] 3′
TsCas12a crRNA
  (SEQ ID NO: 130)
AAUUUCUACUGUUGUAGAU-[spacer] 3′
BsCas12a crRNA
  (SEQ ID NO: 131)
AAUUUCUACUAUUGUAGAU-[spacer] 3′
```

The following sequences are each an example of a scaffold of a naturally existing Cas13a guide RNA (e.g., a scaffold that is 5′ of the guide sequence) (See, e.g., Feng et al., Anal Chem. 2023 Jan. 10; 95(1): 206-217):

```
  (SEQ ID NO: 132)
GUAAGAGACUACCUCUAUAUGAAAGAGGACUAAAAC
(Listeria seeligeri) (“Lse”) (LseCas13a)
  (SEQ ID NO: 133)
GAUAUAGACCACCCCAAUAUCGAAGGGGACUAAAAC
(Leptotrichia shahii) (“Lsh”) (LshCas13a)
  (SEQ ID NO: 134)
AUUUAGACCACCCCAAAAAUGAAGGGGACUAAAAC
(Leptotrichia buccalis) (“Lbu”) (LbuCas13a)
(SEQ ID NO: 135)
GACCACCCCAAAAAUGAAGGGGACUAAAAC
(Leptotrichia buccalis) (“Lbu”) (LbuCas13a)
  (SEQ ID NO: 136)
GAUUUAGACUACCCCAAAAACGAAGGGGACUAAAAC
(LwaCas13a)
  (SEQ ID NO: 137)
GUCACAACUCCCAUGUAGGCGGAGACUGCAAC
(TccCas13a)
(SEQ ID NO: 138)
GGAUUUAGAGUACCCCAAAAAUGAAGGGGACUAAAAC
(LtrCas13a)
```

In some cases, a guide RNA has one or more modifications, e.g., one or more base modifications, one or more backbone modifications (e.g., modified internucleotide linkages), one or more sugar modifications, or any combination thereof. For example, in some cases, a subject guide RNA includes one or more locked nucleic acids (LNAs).

Examples of modified backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable nucleic acids having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

Suitable polynucleotides comprise a sugar substituent group selected from: OH; F; O—, S—, or N-alkyl; O—, S—, or N-alkenyl; O—, S-or N-alkynyl; or O-alkyl-O-alkyl, wherein the alkyl, alkenyl and alkynyl may be substituted or unsubstituted C.sub. 1 to C10 alkyl or C₂ to C₁₀ alkenyl and alkynyl. Particularly suitable are O((CH₂)ₙO)ₘCH₃, O(CH₂)NOCH₃, O(CH₂)ₙNH₂, O(CH₂)ₙCH₃, O(CH₂)NONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. 2′-modified RNA is RNA that has had a modification at the 2′ position of the ribose ring. This modification can increase the stability of the RNA and make it more resistant to nucleases. Examples include, but are not limited to: 2′-O-methylation, and 2′-fluoro-modifications. A suitable modification includes 2′-methoxyethoxy (2′—O—CH₂CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂) 2ON (CH₃) 2 group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido (5,4-b) (1,4) benzoxazin-2 (3H)-one), phenothiazine cytidine (1H-pyrimido (5,4-b) (1,4) benzothiazin-2 (3H)-one), G-clamps such as a substituted phenoxazine cytidine (e.g. 9-(2-aminoethoxy)-H-pyrimido (5,4-(b) (1,4) benzoxazin-2 (3H)-one), carbazole cytidine (2H-pyrimido (4,5-b) indol-2-one), pyridoindole cytidine (H-pyrido (3′,2′: 4,5) pyrrolo (2,3-d) pyrimidin-2-one).

### Error-Prone DNA Polymerases

A number of error-prone DNA polymerases are known in the art, and any known error-prone DNA polymerase is suitable for use in an EvolvR polypeptide of the present disclosure. A suitable error-prone DNA polymerase possesses nick translating activity.

Suitable error-prone DNA polymerases include, but are not limited to, Taq polymerase, Thermus flavus DNA polymerase I, Thermus thermophilus HB-8 DNA polymerase I, Thermophilus ruber DNA polymerase I, Thermophilus brokianus DNA polymerase I, Thermophilus caldophilus GK14 DNA polymerase I, Thermophilus filoformis DNA polymerase I, Bacillus stearothermophilus DNA polymerase I, Bacillus caldotonex YT-G DNA polymerase I, and Bacillus caldovelox YT-F DNA polymerase I. Suitable error-prone DNA polymerases include, but are not limited to, a Niastella koreensis error-prone DNA polymerase, a Mucilaginibacter paludis error-prone DNA polymerase, a Methylobacterium extorquens error-prone DNA polymerase, and a Stenotrophomonas maltophilia error-prone DNA polymerase.

In some cases, a suitable error-prone DNA polymerase comprises an amino acid sequence having 85% or more (e.g., 90% or more, 95% or more, 98% or more, 99% or more, or 100%) amino acid sequence identity to the DNA polymerase I amino acid sequence of any one of SEQ ID NOs: 3-9. In some cases, a suitable error-prone DNA polymerase comprises an amino acid sequence having 85% or more (e.g., 90% or more, 95% or more, 98% or more, 99% or more, or 100%) amino acid sequence identity to the amino acid sequence of any one of SEQ ID NOs: 3-19 (which are Poll, Poll1M (D424A), Poll2M (D424A, I709N), Poll3M (D424A; I709N; A759R), Poll3M-TBD (increases mutagenesis window length), Poll5M, Poll5MΔ (1-325), Phi29 DNA polymerase, T5 DNA polymerase, T7 DNA polymerase, Sequenase, DNA polymerase lota, DNA polymerase n, DNA polymerase K, DNA polymerase 0, DNA polymerase v, and E. coli DNA polymerase IV, respectively).

In some cases, a suitable error-prone DNA polymerase is Escherichia coli DNA polymerase I, with three fidelity-reducing mutations; this error-prone DNA polymerase is referred to as Poll3M. Poll3M comprises D424A, I709N, and A759R substitutions relative to wild-type E. coli DNA polymerase I. In some cases, a suitable error-prone DNA polymerase comprises an amino acid sequence having 85% or more (e.g., 90% or more, 95% or more, 98% or more, 99% or more, or 100%) amino acid sequence identity to the DNA polymerase I amino acid sequence of SEQ ID NO: 3; where the DNA polymerase has an Ala at amino acid position 424, an Asn at amino acid position 709, and an Arg at amino acid position 759 of SEQ ID NO: 3, or a corresponding amino acid in another DNA polymerase.

In some cases, a suitable error-prone DNA polymerase is Escherichia coli DNA polymerase I, with five fidelity-reducing mutations: D242A, I709N, A759R, F742Y, and P796H relative to wild-type E. coli DNA polymerase I. In some cases, a suitable error-prone DNA polymerase comprises an amino acid sequence having 85% or more (e.g., 90% or more, 95% or more, 98% or more, 99% or more, or 100%) amino acid sequence identity to the DNA polymerase I amino acid sequence of SEQ ID NO: 3; where the DNA polymerase has an Ala at amino acid position 242, an Asn at amino acid position 709, an Arg at amino acid position 759, a Tyr at amino acid position 742, and a His at amino acid position 796 of SEQ ID NO: 3; or corresponding amino acids in another DNA polymerase.

In some cases, a suitable error-prone DNA polymerase comprises an amino acid sequence having at least 85%, at least 90%, at least 95%, at least 98%, or at least 99%, amino acid sequence identity to the DNA polymerase I amino acid sequence of SEQ ID NO: 3; where the DNA polymerase has an Ala at amino acid position 424, and an Asn at amino acid position 709.

In some cases, a suitable error-prone DNA polymerase comprises an amino acid sequence having 85% or more (e.g., 90% or more, 95% or more, 98% or more, 99% or more, or 100%) amino acid sequence identity to the Phi29 DNA polymerase amino acid sequence of SEQ ID NO: 10.

In some cases, a suitable error-prone DNA polymerase comprises an amino acid sequence having 85% or more (e.g., 90% or more, 95% or more, 98% or more, 99% or more, or 100%) amino acid sequence identity to the T5 DNA polymerase amino acid sequence of SEQ ID NO: 11.

In some cases, a suitable error-prone DNA polymerase comprises an amino acid sequence having 85% or more (e.g., 90% or more, 95% or more, 98% or more, 99% or more, or 100%) amino acid sequence identity to the T7 DNA polymerase amino acid sequence of SEQ ID NO: 12.

In some cases, a suitable error-prone DNA polymerase comprises an amino acid sequence having 85% or more (e.g., 90% or more, 95% or more, 98% or more, 99% or more, or 100%) amino acid sequence identity to the Sequenase amino acid sequence of SEQ ID NO: 13.

In some cases, a suitable error-prone DNA polymerase comprises an amino acid sequence having 85% or more (e.g., 90% or more, 95% or more, 98% or more, 99% or more, or 100%) amino acid sequence identity to the DNA polymerase iota amino acid sequence of SEQ ID NO: 14.

In some cases, a suitable error-prone DNA polymerase comprises an amino acid sequence having 85% or more (e.g., 90% or more, 95% or more, 98% or more, 99% or more, or 100%) amino acid sequence identity to the DNA polymerase n amino acid sequence of SEQ ID NO: 15.

In some cases, a suitable error-prone DNA polymerase comprises an amino acid sequence having 85% or more (e.g., 90% or more, 95% or more, 98% or more, 99% or more, or 100%) amino acid sequence identity to the DNA polymerase K amino acid sequence of SEQ ID NO: 16.

In some cases, a suitable error-prone DNA polymerase comprises an amino acid sequence having 85% or more (e.g., 90% or more, 95% or more, 98% or more, 99% or more, or 100%) amino acid sequence identity to the DNA polymerase e amino acid sequence of SEQ ID NO: 17.

In some cases, a suitable error-prone DNA polymerase comprises an amino acid sequence having 85% or more (e.g., 90% or more, 95% or more, 98% or more, 99% or more, or 100%) amino acid sequence identity to the DNA polymerase v (nu) amino acid sequence of SEQ ID NO: 18.

In some cases, a suitable error-prone DNA polymerase comprises an amino acid sequence having 85% or more (e.g., 90% or more, 95% or more, 98% or more, 99% or more, or 100%) amino acid sequence identity to the E. coli DNA polymerase IV amino acid sequence of SEQ ID NO: 19.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase iota having the following amino acid sequence:
EKLGVEPEEEGGGDDDEEDAEAWAMELADVGAAASSQGVHDQVLPTPNASSRVIV HVDLDCFYAQVEMISNPELKDKPLGVQQKYLVVTCNYEARKLGVKKLMNVRDAKEK CPQLVLVNGEDLTRYREMSYKVTELLEEFSPVVERLGFDENFVDLTEMVEKRLQQL QSDELSAVTVSGHVYNNQSINLLDVLHIRLLVGSQIAAEMREAMYNQLGLTGCAGVA SNKLLAKLVSGVFKPNQQTVLLPESCQHLIHSLNHIKEIPGIGYKTAKCLEALGINSVR DLQTFSPKILEKELGISVAQRIQKLSFGEDNSPVILSGPPQSFSEEDSFKKCSSEVEAK NKIEELLASLLNRVCQDGRKPHTVRLIIRRYSSEKHYGRESRQCPIPSHVIQKLGTGN YDVMTPMVDILMKLFRNMVNVKMPFHLTLLSVCFCNLKALNTAKKGLIDYYLMPSLST TSRSGKHSFKMKDTHMEDFPKDKETNRDFLPSGRIESTRTRESPLDTTNFSKEKDIN EFPLCSLPEGVDQEVFKQLPVDIQEEILSGKSREKFQGKGSVSCPLHASRGVLSFFS KKQMQDIPINPRDHLSSSKQVSSVSPCEPGTSGFNSSSSSYMSSQKDYSYYLDNRL KDERISQGPKEPQGFHFTNSNPAVSAFHSFPNLQSEQLFSRNHTTDSHKQTVATDS HEGLTENREPDSVDEKITFPSDIDPQVFYELPEAVQKELLAEWKRAGSDFHIGHK (SEQ ID NO: 14); and in some cases has a length of 740 amino acids. In some cases, such a DNA polymerase generates T→G substitutions.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase iota having the following amino acid sequence (amino acids 1-445 of DNA polymerase iota):

(SEQ ID NO: 28)
EKLGVEPEEEGGGDDDEEDAEAWAMELADVGAAASSQGVHDQVLPTPNA
SSRVIVHVDLDCFYAQVEMISNPELKDKPLGVQQKYLVVTCNYEARKLG
VKKLMNVRDAKEKCPQLVLVNGEDLTRYREMSYKVTELLEEFSPVVERL
GFDENFVDLTEMVEKRLQQLQSDELSAVTVSGHVYNNQSINLLDVLHIR
LLVGSQIAAEMREAMYNQLGLTGCAGVASNKLLAKLVSGVFKPNQQTVL
LPESCQHLIHSLNHIKEIPGIGYKTAKCLEALGINSVRDLQTFSPKILE
KELGISVAQRIQKLSFGEDNSPVILSGPPQSFSEEDSFKKCSSEVEAKN
KIEELLASLLNRVCQDGRKPHTVRLIIRRYSSEKHYGRESRQCPIPSHV
IQKLGTGNYDVMTPMVDILMKLFRNMVNVKMPFHLTLLSVCFCNLKALN
TAK;

and in some cases has a length of 445 amino acids. In some cases, such a DNA polymerase generates T→G substitutions. In some cases, such a DNA polymerase has a T→G error rate approaching 1.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase iota having the following amino acid sequence (amino acids 26-445 of DNA polymerase iota):
ELADVGAAASSQGVHDQVLPTPNASSRVIVHVDLDCFYAQVEMISNPELKDKPLGVQ QKYLVVTCNYEARKLGVKKLMNVRDAKEKCPQLVLVNGEDLTRYREMSYKVTELLE EFSPVVERLGFDENFVDLTEMVEKRLQQLQSDELSAVTVSGHVYNNQSINLLDVLHI RLLVGSQIAAEMREAMYNQLGLTGCAGVASNKLLAKLVSGVFKPNQQTVLLPESCQ HLIHSLNHIKEIPGIGYKTAKCLEALGINSVRDLQTFSPKILEKELGISVAQRIQKLSFGE DNSPVILSGPPQSFSEEDSFKKCSSEVEAKNKIEELLASLLNRVCQDGRKPHTVRLIIR RYSSEKHYGRESRQCPIPSHVIQKLGTGNYDVMTPMVDILMKLFRNMVNVKMPFHL TLLSVCFCNLKALNTAK (SEQ ID NO: 29); and in some cases has a length of 419 amino acids. In some cases, such a DNA polymerase generates T→G substitutions. In some cases, such a DNA polymerase has a T→G error rate approaching 1.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase nu (v) having the following amino acid sequence:
ENYEALVGFDLCNTPLSSVAQKIMSAMHSGDLVDSKTWGKSTETMEVINKSSVKYS VQLEDRKTQSPEKKDLKSLRSQTSRGSAKLSPQSFSVRLTDQLSADQKQKSISSLTL SSCLIPQYNQEASVLQKKGHKRKHFLMENINNENKGSINLKRKHITYNNLSEKTSKQ MALEEDTDDAEGYLNSGNSGALKKHFCDIRHLDDWAKSQLIEMLKQAAALVITVMYT DGSTQLGADQTPVSSVRGIVVLVKRQAEGGHGCPDAPACGPVLEGFVSDDPCIYIQI EHSAIWDQEQEAHQQFARNVLFQTMKCKCPVICFNAKDFVRIVLQFFGNDGSWKHV ADFIGLDPRIAAWLIDPSDATPSFEDLVEKYCEKSITVKVNSTYGNSSRNIVNQNVRE NLKTLYRLTMDLCSKLKDYGLWQLFRTLELPLIPILAVMESHAIQVNKEEMEKTSALLG ARLKELEQEAHFVAGERFLITSNNQLREILFGKLKLHLLSQRNSLPRTGLQKYPSTSE AVLNALRDLHPLPKIILEYRQVHKIKSTFVDGLLACMKKGSISSTWNQTGTVTGRLSA KHPNIQGISKHPIQITTPKNFKGKEDKILTISPRAMFVSSKGHTFLAADFSQIELRILTHL SGDPELLKLFQESERDDVFSTLTSQWKDVPVEQVTHADREQTKKVVYAVVYGAGKE RLAACLGVPIQEAAQFLESFLQKYKKIKDFARAAIAQCHQTGCVVSIMGRRRPLPRIH AHDQQLRAQAERQAVNFVVQGSAADLCKLAMIHVFTAVAASHTLTARLVAQIHDELL FEVEDPQIPECAALVRRTMESLEQVQALELQLQVPLKVSLSAGRSWGHLVPLQEAW GPPPGPCRTESPSNSLAAPGSPASTQPPPLHFSPSFCL (SEQ ID NO: 18); and in some cases has a length of 899 amino acids. In some cases, such a DNA polymerase generates G->T substitutions.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase eta (n) having the following amino acid sequence:
ATGQDRVVALVDMDCFFVQVEQRQNPHLRNKPCAVVQYKSWKGGGIIAVSYEARA FGVTRSMWADDAKKLCPDLLLAQVRESRGKANLTKYREASVEVMEIMSRFAVIERA SIDEAYVDLTSAVQERLQKLQGQPISADLLPSTYIEGLPQGPTTAEETVQKEGMRKQ GLFQWLDSLQIDNLTSPDLQLTVGAVIVEEMRAAIERETGFQCSAGISHNKVLAKLAC GLNKPNRQTLVSHGSVPQLFSQMPIRKIRSLGGKLGASVIEILGIEYMGELTQFTESQ LQSHFGEKNGSWLYAMCRGIEHDPVKPRQLPKTIGCSKNFPGKTALATREQVQWW LLQLAQELEERLTKDRNDNDRVATQLVVSIRVQGDKRLSSLRRCCALTRYDAHKMS HDAFTVIKNCNTSGIQTEWSPPLTMLFLCATKFSASAPSSSTDITSFLSSDPSSLPKVP VTSSEAKTQGSGPAVTATKKATTSLESFFQKAAERQKVKEASLSSLTAPTQAPMSNS PSKPSLPFQTSQSTGTEPFFKQKSLLLKQKQLNNSSVSSPQQNPWSNCKALPNSLP TEYPGCVPVCEGVSKLEESSKATPAEMDLAHNSQSMHASSASKSVLEVTQKATPNP SLLAAEDQVPCEKCGSLVPVWDMPEHMDYHFALELQKSFLQPHSSNPQVVSAVSH QGKRNPKSPLACTNKRPRPEGMQTLESFFKPLTH (SEQ ID NO: 15); and in some cases has a length of 713 amino acids.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase eta (n) having the following amino acid sequence:
ATGQDRVVALVDMDCFFVQVEQRQNPHLRNKPCAVVQYKSWKGGGIIAVSYEARA FGVTRSMWADDAKKLCPDLLLAQVRESRGKANLTKYREASVEVMEIMSRFAVIERA SIDEAYVDLTSAVQERLQKLQGQPISADLLPSTYIEGLPQGPTTAEETVQKEGMRKQ GLFQWLDSLQIDNLTSPDLQLTVGAVIVEEMRAAIERETGFQCSAGISHNKVLAKLAC GLNKPNRQTLVSHGSVPQLFSQMPIRKIRSLGGKLGASVIEILGIEYMGELTQFTESQ LQSHFGEKNGSWLYAMCRGIEHDPVKPRQLPKTIGCSKNFPGKTALATREQVQWW LLQLAQELEERLTKDRNDNDRVATQLVVSIRVQGDKRLSSLRRCCALTRYDAHKMS HDAFTVIKNCNTSGIQTEWSPPLTMLFLCATKFSASAPSSSTDITSFLSSDPSSLPKVP VTSSEAKTQGSGPAVTATKKATTSLESFFQKAAERQKVKEASLSSLTAPTQAPMSN (SEQ ID NO: 48); and in some cases has a length of 511 amino acids.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase theta (e) having the following amino acid sequence:

(SEQ ID NO: 17)
NLLRRSGKRRRSESGSDSFSGSGGDSSASPQFLSGSVLSPPPGLGRCLKAAAAGE
CKPTVPDYERDKLLLANWGLPKAVLEKYHSFGVKKMFEWQAECLLLGQVLEGKNLV
YSAPTSAGKTLVAELLILKRVLEMRKKALFILPFVSVAKEKKYYLQSLFQEVGIKVDGY
MGSTSPSRHFSSLDIAVCTIERANGLINRLIEENKMDLLGMVVVDELHMLGDSHRGYL
LELLLTKICYITRKSASCQADLASSLSNAVQIVGMSATLPNLELVASWLNAELYHTDFR
PVPLLESVKVGNSIYDSSMKLVREFEPMLQVKGDEDHVVSLCYETICDNHSVLLFCP
SKKWCEKLADIIAREFYNLHHQAEGLVKPSECPPVILEQKELLEVMDQLRRLPSGLDS
VLQKTVPWGVAFHHAGLTFEERDIIEGAFRQGLIRVLAATSTLSSGVNLPARRVIIRTP
IFGGRPLDILTYKQMVGRAGRKGVDTVGESILICKNSEKSKGIALLQGSLKPVRSCLQ
RREGEEVTGSMIRAILEIIVGGVASTSQDMHTYAACTFLAASMKEGKQGIQRNQESV
QLGAIEACVMWLLENEFIQSTEASDGTEGKVYHPTHLGSATLSSSLSPADTLDIFADL
QRAMKGFVLENDLHILYLVTPMFEDWTTIDWYRFFCLWEKLPTSMKRVAELVGVEE
GFLARCVKGKVVARTERQHRQMAIHKRFFTSLVLLDLISEVPLREINQKYGONRGQIQ
SLQQSAAVYAGMITVFSNRLGWHNMELLLSQFQKRLTFGIQRELCDLVRVSLLNAQR
ARVLYASGFHTVADLARANIVEVEVILKNAVPFKSARKAVDEEEEAVEERRNMRTIW
VTGRKGLTEREAAALIVEEARMILQQDLVEMGVQWNPCALLHSSTCSLTHSESEVKE
HTFISQTKSSYKKLTSKNKSNTIFSDSYIKHSPNIVQDLNKSREHTSSFNCNFQNGNQ
EHQTCSIFRARKRASLDINKEKPGASQNEGKTSDKKVVQTFSQKTKKAPLNFNSEKM
SRSFRSWKRRKHLKRSRDSSPLKDSGACRIHLQGQTLSNPSLCEDPFTLDEKKTEF
RNSGPFAKNVSLSGKEKDNKTSFPLQIKQNCSWNITLTNDNFVEHIVTGSQSKNVTC
QATSVVSEKGRGVAVEAEKINEVLIQNGSKNQNVYMKHHDIHPINQYLRKQSHEQTS
TITKQKNIIERQMPCEAVSSYINRDSNVTINCERIKLNTEENKPSHFQALGDDISRTVIP
SEVLPSAGAFSKSEGQHENFLNISRLQEKTGTYTTNKTKNNHVSDLGLVLCDFEDSF
YLDTQSEKIIQQMATENAKLGAKDTNLAAGIMQKSLVQQNSMNSFQKECHIPFPAEQ
HPLGATKIDHLDLKTVGTMKQSSDSHGVDILTPESPIFHSPILLEENGLFLKKNEVSVT
DSQLNSFLQGYQTQETVKPVILLIPQKRTPTGVEGECLPVPETSLNMSDSLLFDSFSD
DYLVKEQLPDMQMKEPLPSEVTSNHFSDSLCLQEDLIKKSNVNENQDTHQQLTCSN
DESIIFSEMDSVQMVEALDNVDIFPVQEKNHTVVSPRALELSDPVLDEHHQGDQDGG
DQDERAEKSKLTGTRQNHSFIWSGASFDLSPGLQRILDKVSSPLENEKLKSMTINFS
SLNRKNTELNEEQEVISNLETKQVQGISFSSNNEVKSKIEMLENNANHDETSSLLPRK
ESNIVDDNGLIPPTPIPTSASKLTFPGILETPVNPWKTNNVLQPGESYLFGSPSDIKNH
DLSPGSRNGFKDNSPISDTSFSLQLSQDGLQLTPASSSSESLSIIDVASDQNLFQTFIK
EWRCKKRFSISLACEKIRSLTSSKTATIGSRFKQASSPQEIPIRDDGFPIKGCDDTLVV
GLAVCWGGRDAYYFSLQKEQKHSEISASLVPPSLDPSLTLKDRMWYLQSCLRKESD
KECSVVIYDFIQSYKILLLSCGISLEQSYEDPKVACWLLDPDSQEPTLHSIVTSFLPHEL
PLLEGMETSQGIQSLGLNAGSEHSGRYRASVESILIFNSMNQLNSLLQKENLQDVFR
KVEMPSQYCLALLELNGIGFSTAECESQKHIMQAKLDAIETQAYQLAGHSFSFTSSD
DIAEVLFLELKLPPNREMKNQGSKKTLGSTRRGIDNGRKLRLGRQFSTSKDVLNKLK
ALHPLPGLILEWRRITNAITKVVFPLQREKCLNPFLGMERIYPVSQSHTATGRITFTEP
NIQNVPRDFEIKMPTLVGESPPSQAVGKGLLPMGRGKYKKGFSVNPRCQAQMEER
AADRGMPFSISMRHAFVPFPGGSILAADYSQLELRILAHLSHDRRLIQVLNTGADVFR
SIAAEWKMIEPESVGDDLRQQAKQICYGIIYGMGAKSLGEQMGIKENDAACYIDSFKS
RYTGINQFMTETVKNCKRDGFVQTILGRRRYLPGIKDNNPYRKAHAERQAINTIVQG
SAADIVKIATVNIQKQLETFHSTFKSHGHREGMLQSDQTGLSRKRKLQGMFCPIRGG
FFILQLHDELLYEVAEEDVVQVAQIVKNEMESAVKLSVKLKVKVKIGASWGELKDFDV.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase kappa (κ) having the following amino acid sequence:
DSTKEKCDSYKDDLLLRMGLNDNKAGMEGLDKEKINKIIMEATKGSRFYGNELKKEK QVNQRIENMMQQKAQITSQQLRKAQLQVDRFAMELEQSRNLSNTIVHIDMDAFYAA VEMRDNPELKDKPIAVGSMSMLSTSNYHARRFGVRAAMPGFIAKRLCPQLIIVPPNF DKYRAVSKEVKEILADYDPNFMAMSLDEAYLNITKHLEERQNWPEDKRRYFIKMGSS VENDNPGKEVNKLSEHERSISPLLFEESPSDVQPPGDPFQVNFEEQNNPQILQNSVV FGTSAQEVVKEIRFRIEQKTTLTASAGIAPNTMLAKVCSDKNKPNGQYQILPNRQAVM DFIKDLPIRKVSGIGKVTEKMLKALGIITCTELYQQRALLSLLFSETSWHYFLHISLGLG STHLTRDGERKSMSVERTFSEINKAEEQYSLCQELCSELAQDLQKERLKGRTVTIKL KNVNFEVKTRASTVSSVVSTAEEIFAIAKELLKTEIDADFPHPLRLRLMGVRISSFPNE EDRKHQQRSIIGFLQAGNQALSATECTLEKTDKDKFVKPLEMSHKKSFFDKKRSERK WSHQDTFKCEAVNKQSFQTSQPFQVLKKKMNENLEISENSDDCQILTCPVCFRAQG CISLEALNKHVDECLDGPSISENFKMFSCSHVSATKVNKKENVPASSLCEKQDYEAH PKIKEISSVDCIALVDTIDNSSKAESIDALSNKHSKEECSSLPSKSFNIEHCHQNSSSTV SLENEDVGSFRQEYRQPYLCEVKTGQALVCPVCNVEQKTSDLTLFNVHVDVCLNKS FIQELRKDKFNPVNQPKESSRSTGSSSGVQKAVTRTKRPGLMTKYSTSKKIKPNNPK HTLDIFFK (SEQ ID NO: 16); and in some cases has a length of 870 amino acids.

In some cases, a suitable error-prone DNA polymerase comprises an amino acid sequence having 85% or more (e.g., 90% or more, 95% or more, 98% or more, 99% or more, or 100%) amino acid sequence identity to a DNA polymerase kappa (κ) having the following amino acid sequence:
DSTKEKCDSYKDDLLLRMGLNDNKAGMEGLDKEKINKIIMEATKGSRFYGNELKKEK QVNQRIENMMQQKAQITSQQLRKAQLQVDRFAMELEQSRNLSNTIVHIDMDAFYAA VEMRDNPELKDKPIAVGSMSMLSTSNYHARRFGVRAAMPGFIAKRLCPQLIIVPPNF DKYRAVSKEVKEILADYDPNFMAMSLDEAYLNITKHLEERQNWPEDKRRYFIKMGSS VENDNPGKEVNKLSEHERSISPLLFEESPSDVQPPGDPFQVNFEEQNNPQILQNSVV FGTSAQEVVKEIRFRIEQKTTLTASAGIAPNTMLAKVCSDKNKPNGQYQILPNRQAVM DFIKDLPIRKVSGIGKVTEKMLKALGIITCTELYQQRALLSLLFSETSWHYFLHISLGLG STHLTRDGERKSMSVERTFSEINKAEEQYSLCQELCSELAQDLQKERLKGRTVTIKL KNVNFEVKTRASTVSSVVSTAEEIFAIAKELLKTEIDADFPHPLRLRLMGVRISSFPNE EDRKHQQRSIIGFLQAGNQALSATECTLEKTDKDKFVKPLE (SEQ ID NO: 49); and in some cases has a length of 560 amino acids.

In some cases, the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 8. In some cases, the error-prone DNA polymerase comprises an amino acid sequence having 95% or more (e.g., 98% or more, 99% or more, or 100%) amino acid sequence identity to the truncated Poll5M nucleic acid sequence of SEQ ID NO: 8. In some cases, the error-prone DNA polymerase comprises the amino acid sequence of SEQ ID NO: 8.

In some cases, the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 9. In some cases, the error-prone DNA polymerase comprises an amino acid sequence having 95% or more (e.g., 98% or more, 99% or more, or 100%) amino acid sequence identity to the truncated Poll5M nucleic acid sequence of SEQ ID NO: 9. In some cases, the error-prone DNA polymerase comprises the amino acid sequence of SEQ ID NO: 9.

In some cases, the Slug-nCas9 of a subject EvolvR polypeptide is an NNGR-Slug-nCas9 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 8. In some cases, the Slug-nCas9 of a subject EvolvR polypeptide is an NNG-Slug-nCas9 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 8.

In some cases, the Slug-nCas9 of a subject EvolvR polypeptide is an NNGR-Slug-nCas9 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 9. In some cases, the Slug-nCas9 of a subject EvolvR polypeptide is an NNG-Slug-nCas9 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 9.

In some cases, the Slug-nCas9 of a subject EvolvR polypeptide includes an amino acid sequence having 85% or more (e.g., 90% or more, 95% or more, 98% or more, 99% or more, or 100%) amino acid sequence identity to SEQ ID NO: 1 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 8. In some cases, the Slug-nCas9 of a subject EvolvR polypeptide includes an amino acid sequence having 85% or more (e.g., 90% or more, 95% or more, 98% or more, 99% or more, or 100%) amino acid sequence identity to SEQ ID NO: 2 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 8.

In some cases, the Slug-nCas9 of a subject EvolvR polypeptide includes an amino acid sequence having 85% or more (e.g., 90% or more, 95% or more, 98% or more, 99% or more, or 100%) amino acid sequence identity to SEQ ID NO: 1 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 9. In some cases, the Slug-nCas9 of a subject EvolvR polypeptide includes an amino acid sequence having 85% or more (e.g., 90% or more, 95% or more, 98% or more, 99% or more, or 100%) amino acid sequence identity to SEQ ID NO: 2 and the error-prone DNA polymerase comprises an amino acid sequence having 85% or more (e.g., 90% or more, 95% or more, 98% or more, 99% or more, or 100%) amino acid sequence identity to the truncated Poll5M nucleic acid sequence of SEQ ID NO: 9.

### Dimerization Pair (Dimerizing Pair)

In some cases, the CRISPR-Cas effector polypeptide (a nickase that cleaves one strand of a target DNA, e.g., a Slug-nCas9) is fused to one member (a first member, i.e., a first fusion partner) of a dimerization pair and the error-prone DNA polymerase (e.g., Poll5MΔ (1-325), e.g., SEQ ID NO: 9) is fused to the other member (the second member, i.e., a second fusion partner) of the dimerization pair. The first fusion partner of the CRISPR-Cas effector polypeptide (e.g., a Slug-nCas9), and the second fusion partner of the error-prone DNA polymerase constitute a “dimer pair” (also referred to as a “dimerizing pair” or a “dimerization pair”).

A dimerization pair is a pair of polypeptides (e.g., protein domains) that dimerize with one another. Each member of the dimerization pair can be fused to a protein, e.g., ‘member 1’ (i.e., a first fusion partner) of a given dimerization pair can be fused to a ‘protein 1’ while ‘member 2’ (i.e., a second fusion partner) of the dimerization pair can be fused to a ‘protein 2’, and dimerization of member 1 with member 2 therefore brings protein 1 and protein 2 together. In other words, each member (each polypeptide) of the dimer pair can be part of a different polypeptide, and when the members of the binding pair (the dimer pair) are brought into close proximity with one another (e.g., bind to/dimerize with one another), the two different polypeptides (heterologous polypeptides) to which the dimer pair members are fused are brought into proximity with one another and can be said to dimerize (i.e., as a consequence of the members of the dimer pair dimerizing). As an example, in some cases a dimerization domain (the “first” dimerization domain of a pair) may be any engineered domain that orthogonally binds to another dimerization domain (the “second” dimerization domain of the pair).

In some cases, the members of a dimerization pair bind constitutively (they will bind to one another as long as they are both present and have access to one another) and the pair can be referred to as a constitutive dimerization pair. For example, constitutive domains/elements can associate with their binding partner under suitable conditions (e.g., physiologically constitutive dimerization domains/elements will dimerize under physiologic conditions without the need for introduction of an initiator of dimerization). As such, a dimerization inducer (a dimerizing agent) is not required for dimerization of constitutive dimerization pairs.

In some cases, dimerization of the dimerization pair is inducible (they will not bind to one another in the absence of an inducer even when both are present and have access to one another) and the pair can be referred to as an inducible dimerization pair. The inducer of an inducible dimerization pair can be referred to as a dimerizing agent.

The member can be fused at any convenient location. For example, in some cases it is fused N-terminal to the CRISPR-Cas effector polypeptide (e.g., Slug-nCas9), e.g., at or near (e.g., within 50 amino acids) the N-terminus of the CRISPR-Cas effector polypeptide (e.g., Slug-nCas9). In some cases, it is fused N-terminal to the error-prone DNA polymerase, e.g., at or near (e.g., within 50 amino acids) the N-terminus of the error-prone DNA polymerase. In some cases it is fused C-terminal to the CRISPR-Cas effector polypeptide (e.g., Slug-nCas9), e.g., at or near (e.g., within 50 amino acids) the C-terminus of the CRISPR-Cas effector polypeptide (e.g., Slug-nCas9). In some cases, it is fused C-terminal to the error-prone DNA polymerase, e.g., at or near (e.g., within 50 amino acids) the C-terminus of the error-prone DNA polymerase.

### Constitutive Dimerization Pairs

As noted above, in some embodiments, the members of a dimerization pair bind constitutively (they will bind to one another as long as they are both present and have access to one another) and the pair can be referred to as a constitutive dimerization pair. Many such pairs will be known to one of ordinary skill in the art and any convenient pair can be used.

Examples of pairs of dimerization domains include, but are not limited to, helix-turn-helix-based designed heterodimers (DHDs) (e.g., those described in Chen et al., Nature. 2019 565:106-111), heterospecific synthetic coiled-coil synthetic leucine zippers (synZIPs) (e.g., those described in, e.g., Thompson et al., ACS Synth. Biol. 2012 1:118-29; Reinke et al., JACS 2010 132:6025-31; and Cho et al Cell 2018 173:1426-1438), miniproteins (Nature 2017 550:74-79), intrabodies (Chen et al Human Gene Therapy 1994 5:595-601). scFvs, nanobodies, Fabs, DARPins and monobodies could also be used, among many others. For example, if synZIPs are used, one dimerization domain may be BZip (RR) and the other one may be AZip (EE). SYNZIP 1 to SYNZIP 48, and BATF, FOS, ATF4, ATF3, BACHI, JUND, NFE2L3, and HEPTAD may be used in some cases. Examples also include SpyCatcher and SpyTag (and all possible variations of same, as would be known to one of ordinary skill in the art), as well as GFP-10/GFP11 (see, e.g., Hatlem et al., Int J Mol Sci. 2019 Apr. 30; 20 (9): 2129; and Kamiyama et al., Nat Commun. 2016 Mar. 18; 7:11046).

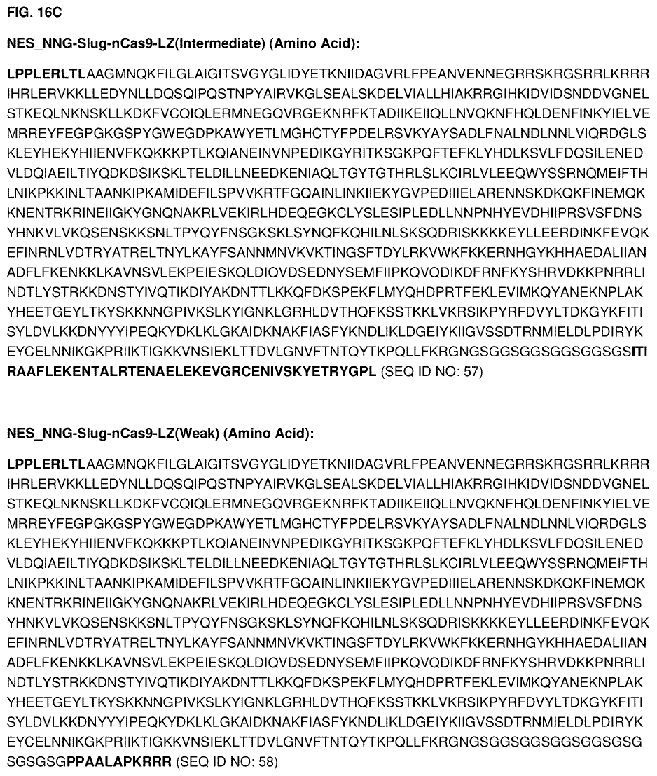

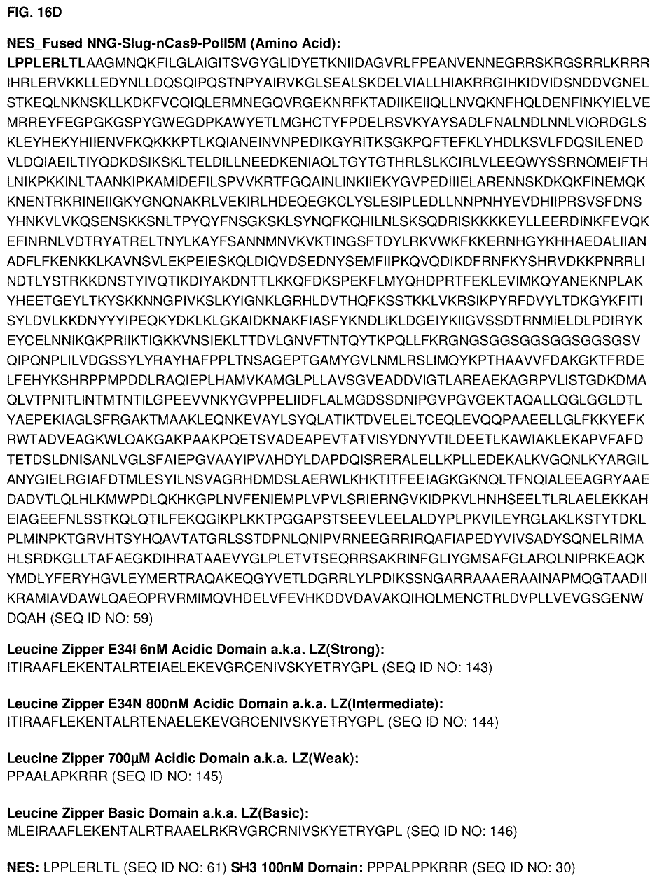

Examples of constitutive dimerization domains to be used as part of a constitutive dimerization pair include, but are not limited to, leucine zipper polypeptides such as (see, e.g., FIG. 16):
Leucine Zipper E34| 6 nM Acidic Domain a.k.a. LZ (Strong):

(SEQ ID NO: 143)
ITIRAAFLEKENTALRTEIAELEKEVGRCENIVSKYETRYGPL
(also listed as SEQ ID NO: 51)

Leucine Zipper E34N 800 nM Acidic Domain a.k.a. LZ (Intermediate):

(SEQ ID NO: 144)
ITIRAAFLEKENTALRTENAELEKEVGRCENIVSKYETRYGPL
(also listed as SEQ ID NO: 52)

Leucine Zipper 700 μM Acidic Domain a.k.a. LZ (Weak):

(SEQ ID NO: 145)
PPAALAPKRRR (also listed as SEQ ID NO: 53)

Leucine Zipper Basic Domain a.k.a. LZ (Basic):

(SEQ ID NO: 146)
MLEIRAAFLEKENTALRTRAAELRKRVGRCRNIVSKYETRYGPL
(also listed as SEQ ID NO: 54)

Illustrative examples of Slug-nCas9 proteins and a DNA-polymerase fused to the above sequences are presented in FIG. 16. See, e.g., SEQ ID NOs: 55-58. These proteins also include an optional N-terminal NES sequence (i.e., an NES can be present in some cases and absent in some cases).

In some cases, each member of the binding pair is a coiled-coil domain. Examples of suitable coiled-coil domains include, but are not limited to:

SYNZIP14:
(SEQ ID NO: 147)
NDLDAYEREAEKLEKKNEVLRNRLAALENELATLRQEVASMKQELQS
SYNZIP17:
(SEQ ID NO: 148)
NEKEELKSKKAELRNRIEQLKQKREQLKQKIANLRKEIEAYK
SYNZIP18:
(SEQ ID NO: 149)
SIAATLENDLARLENENARLEKDIANLERDLAKLEREEAYF

In some embodiments, the first dimerization domain binds to the target dimerization with a low affinity. In these embodiments, the first dimerization domain may bind to the target dimerization with a Kd of greater than 10 nM (e.g., a K the range of 50 nM to 1000 nM, or at least 100 nM to 500 nM), as measured by the method of Thompson (ACS Synth. Biol. 2012 1:118-29).

Dimerization domains that bind to one another with a low affinity can be engineered from high affinity interactions relatively straightforwardly. For example, domains that interact with one another with a high affinity may be modified to decrease the affinity of the interaction. Such methods include, but are not limited to e.g., random (untargeted) and targeted (directed) mutagenesis, alanine scanning, and screening (e.g., phage display, etc.) methods. For example, synZIPs are around 30 amino acids in length and are composed of eight a-helical turns, with 5 leucines that spaced every 7 aa. To decrease the affinity of synZIP, one could remove 7 residues at a time from the ends to remove a single heptad repeat at a time. As such, if a low affinity synZIP is used one of the synthetic leucine zipper domains may have up to seven a-helical turns (e.g., 5, 6, or 7 turns), not the full complement of eight a-helical turns.

A skilled artisan will recognize that many heterologous domains whose associations are constitutive are well known in the art. Examples described in the art include, but are not limited to, heterodimerization of PDZ domains from the mammalian proteins neuronal nitric oxide synthase (nNOS) and syntrophin (Ung et al. (2001) EMBO J. 20:3728-3737; herein incorporated by reference in its entirety), heterodimerization of the Xenopus XLIM1 and LDB1 proteins (Ung et al. (2001) EMBO J. 20:3728-3737; herein incorporated by reference in its entirety), oligomerization of RFG (also named ELE1 or ARA70) through its coiled-coil domain (Monaco et al. (2001) Oncogene 20:599-608; herein incorporated by reference in its entirety), oligomerization of the leucine zipper domain of yeast GCN4 (Harbury et al. (1993) Science 262:1401-1407; herein incorporated by reference in its entirety), and oligomerization of the TEL helix-loop-helix (HLH) domain (Golub et al. (1996) Mol. Cell. Biol. 16, 4107-4116; herein incorporated by reference in its entirety) and their variants.

In some instances, the affinity of a dimerization pair may be assessed, estimated, and/or quantitated in various ways. For example, in some instances, affinity may be assessed, estimated, and/or quantitated by a biochemical or biophysical method. Useful methods for assessing, estimating, and/or determining absolute and/or relative and/or estimated affinities may include but are not limited to e.g., affinity electrophoresis, bimolecular fluorescence complementation (BiFC), bio-layer interferometry, co-immunoprecipitation, dual polarization interferometry (DPI), dynamic light scattering (DLS), flow-induced dispersion analysis (FIDA), fluorescence correlation spectroscopy, fluorescence polarization/anisotropy, fluorescence resonance energy transfer (FRET), isothermal titration calorimetry (ITC), microscale thermophoresis (MST), phage display, proximity ligation assay (PLA), quantitative immunoprecipitation combined with knock-down (QUICK), rotating cell-based ligand binding assay, static light scattering (SLS), single colour reflectometry (SCORE), surface plasmon resonance (SPR), tandem affinity purification (TAP), and the like.

Examples of dimerization domains also include: PDZ domains from the mammalian proteins neuronal nitric oxide synthase (nNOS) and syntrophin.

GTPase Binding Domain (GBD) from the actin polymerization switch N-WASP is yet another example of a dimerization domain that can be used. Moreover, in some cases, any computationally designed protein-protein interaction domain may also be used.

### Inducible Dimerization Pairs

In some cases, the dimerization pair is inducible, i.e., the members of the pair can be induced to dimerize by a dimerizing agent (also referred to as a dimerizer). In the absence of the dimerizing agent, the members of an inducible dimerization pair do not dimerize. An inducible dimerization pair can also be referred to as a dimerizer-binding pair (i.e., the members of the dimerization pair bind to one another in the presence of a dimerizer). In some cases, the first and second members of the pair bind to a different site of the same molecule (a “dimerizer”). In the presence of a dimerizer, both members of the dimerizer-binding pair bind to a different site of the dimerizer and are thus brought into proximity with one another. In some embodiments, binding to the dimerizer is reversible. In some embodiments, binding to the dimerizer is irreversible. In some embodiments, binding to the dimerizer is non-covalent. In some embodiments, binding to the dimerizer is covalent.

Dimer pairs suitable for use include dimerizer-binding pairs that dimerize upon binding of a first member of a dimer pair to a dimerizing agent, where the dimerizing agent induces a conformational change in the first member of the dimer pair, and where the conformational change allows the first member of the dimer pair to bind (covalently or non-covalently) to a second member of the dimer pair. Dimer pairs suitable for use include dimer pairs in which exposure to light (e.g., blue light) induces dimerization of the dimer pair. Regardless of the mechanism, the dimer pair will dimerize upon exposure to an agent that induces dimerization, where the agent is in some cases a small molecule, or, in other cases, light. Thus, for simplicity, the discussion below referring to “dimerizer-binding pairs” includes dimer pairs that dimerize regardless of the mechanism.

Examples of inducible dimerization pairs (e.g., dimerizer-binding pairs) include, but are not limited to:
(a) FKBP1A (FK506 binding protein) (e.g., a rapamycin binding portion) paired with FKBP1A (e.g., a rapamycin binding portion): dimerization induced by rapamycin and/or rapamycin analogs known as rapalogs;
(b) FKBP1A (e.g., a rapamycin binding portion) paired with FRB (Fkbp-Rapamycin Binding Domain): dimerization induced by rapamycin and/or rapamycin analogs known as rapalogs;
(c) FKBP1A (e.g., a rapamycin binding portion) paired with CnA (calcineurin catalytic subunit A): dimerization induced by rapamycin and/or rapamycin analogs known as rapalogs;
(d) FKBP1A (e.g., a rapamycin binding portion) paired with cyclophilin: dimerization induced by rapamycin and/or rapamycin analogs known as rapalogs;
(e) GyrB (Gyrase B) paired with GyrB: dimerization induced by coumermycin;
(f) DHFR (dihydrofolate reductase) paired with DHFR: dimerization induced by methotrexate);
(g) DmrB paired with DmrB: dimerization induced by AP20187;
(h) PYL paired with ABI: dimerization induced by abscisic acid;
(i) Cry2 paired with CIB1: dimerization induced by blue light; and
(j) GAI paired with GID1: dimerization induced by gibberellin.

A first or a second member of a dimer (e.g., a dimerizer-binding pair) of a subject EvolvR polypeptide can have a length of from about 50 amino acids to about 300 amino acids or more; e.g., a first or a second member of a dimer (e.g., a dimerizer-binding pair) of a subject EvolvR polypeptide can have a length of from about 50 aa to about 100 aa, from about 100 aa to about 150 aa, from about 150 aa to about 200 aa, from about 200 aa toa bout 250 aa, from about 250 aa to about 300 aa, or more than 300 aa.

In some cases, a member of an inducible dimer pair (e.g., a dimerizer-binding pair) of a subject EvolvR polypeptide is derived from FKBP (i.e., FKBP1A). For example, a suitable dimerizer-binding pair member can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: 150)
MGVQVETISPGDGRTFPKRGQTCVVHYTGMLEDGKKFDSSRDRNKPFKF
MLGKQEVIRGWEEGVAQMSVGQRAKLTISPDYAYGATGHPGIIPPHATL
VFDVELLKLE.

In some cases, a member of a dimerizer-binding pair of a subject EvolvR polypeptide is derived from calcineurin catalytic subunit A (also known as PPP3CA; CALN; CALNA; CALNA1; CCN1; CNA1; PPP2B; CAM-PRP catalytic subunit; calcineurin A alpha; calmodulin-dependent calcineurin A subunit alpha isoform; protein phosphatase 2B, catalytic subunit, alpha isoform; etc.). For example, a suitable dimerizer-binding pair member can comprise an amino acid sequence having at least about 75%, at least about 80%, at least about 85%, at least about 90%, at least about 95%, at least about 98%, or 100% amino acid sequence identity to the following amino acid sequence (PP2Ac domain):

(SEQ ID NO: 151)
LEESVALRIITEGASILRQEKNLLDIDAPVTVCGDIHGQFFDLMKLFEVGGSPANTRYL
FLGDYVDRGYFSIECVLYLWALKILYPKTLFLLRGNHECRHLTEYFTFKQECKIKYSER
VYDACMDAFDCLPLAALMNQQFLCVHGGLSPEINTLDDIRKLDRFKEPPAYGPMCDI
LWSDPLEDFGNEKTQEHFTHNTVRGCSYFYSYPAVCEFLQHNNLLSILRAHEAQDA
GYRMYRKSQTTGFPSLITIFSAPNYLDVYNNKAAVLKYENNVMNIRQFNCSPHPYWL
PNFM.

In some cases, a member of an inducible dimer pair (e.g., a dimerizer-binding pair) is derived from cyclophilin (also known cyclophilin A, PPIA, CYPA, CYPH, PPlase A, etc.). For example, a suitable dimerizer-binding pair member can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: 152)
MVNPTVFFDIAVDGEPLGRVSFELFADKVPKTAENFRALSTGEKGFGYKGSCFHRIIP
GFMCQGGDFTRHNGTGGKSIYGEKFEDENFILKHTGPGILSMANAGPNTNGSQFFIC
TAKTEWLDGKHVVFGKVKEGMNIVEAMERFGSRNGKTSKKITIADCGQLE.

In some cases, a member of an inducible dimer pair (e.g., a dimerizer-binding pair) is derived from MTOR (also known as FKBP-rapamycin associated protein; FK506 binding protein 12-rapamycin associated protein 1; FK506 binding protein 12-rapamycin associated protein 2; FK506-binding protein 12-rapamycin complex-associated protein 1; FRAP; FRAP1; FRAP2; RAFT1; and RAPT1). For example, a suitable dimerizer-binding pair member can comprise an amino acid sequence having at least about 75%, at least about 80%, at least about 85%, at least about 90%, at least about 95%, at least about 98%, or 100% amino acid sequence identity to the following amino acid sequence (also known as “Frb”: Fkbp-Rapamycin Binding Domain):

(SEQ ID NO: 153)
MILWHEMWHEGLEEASRLYFGERNVKGMFEVLEPLHAMMERGPQTLKET
SFNQAYGRDLMEAQEWCRKYMKSGNVKDLLQAWDLYYHVFRRISK.

In some cases, a member of an inducible dimer pair (e.g., a dimerizer-binding pair) is derived from GyrB (also known as DNA gyrase subunit B). For example, a suitable dimerizer-binding pair member can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 200 amino acids (aa), from about 200 aa to about 300 aa, from about 300 aa to about 400 aa, from about 400 aa to about 500 aa, from about 500 aa to about 600 aa, from about 600 aa to about 700 aa, or from about 700 aa to about 800 aa, of the following GyrB amino acid sequence from Escherichia coli (or to the DNA gyrase subunit B sequence from any organism):
MSNSYDSSSIKVLKGLDAVRKRPGMYIGDTDDGTGLHHMVFEVVDNAIDEALAGHC KEIIVTIHADNSVSVQDDGRGIPTGIHPEEGVSAAEVIMTVLHAGGKFDDNSYKVSGG LHGVGVSVVNALSQKLELVIQREGKIHRQIYEHGVPQAPLAVTGETEKTGTMVRFWP SLETFTNVTEFEYEILAKRLRELSFLNSGVSIRLRDKRDGKEDHFHYEGGIKAFVEYLN KNKTPIHPNIFYFSTEKDGIGVEVALQWNDGFQENIYCFTNNIPQRDGGTHLAGFRAA MTRTLNAYMDKEGYSKKAKVSATGDDAREGLIAVVSVKVPDPKFSSQTKDKLVSSE VKSAVEQQMNELLAEYLLENPTDAKIVVGKIIDAARAREAARRAREMTRRKGALDLA GLPGKLADCQERDPALSELYLVEGDSAGGSAKQGRNRKNQAILPLKGKILNVEKARF DKMLSSQEVATLITALGCGIGRDEYNPDKLRYHSIIIMTDADVDGSHIRTLLLTFFYRQ MPEIVERGHVYIAQPPLYKVKKGKQEQYIKDDEAMDQYQISIALDGATLHTNASAPAL AGEALEKLVSEYNATQKMINRMERRYPKAMLKELIYQPTLTEADLSDEQTVTRWVNA LVSELNDKEQHGSQWKFDVHTNAEQNLFEPIVRVRTHGVDTDYPLDHEFITGGEYR RICTLGEKLRGLLEEDAFIERGERRQPVASFEQALDWLVKESRRGLSIQRYKGLGEM NPEQLWETTMDPESRRMLRVTVKDAIAADQLFTTLMGDAVEPRRAFIEENALKAANI DI (SEQ ID NO: 154). In some cases, a member of a dimerizer-binding pair comprises an amino acid sequence having at least about 75%, at least about 80%, at least about 85%, at least about 90%, at least about 95%, at least about 98%, or 100% amino acid sequence identity to amino acids 1-220 of the above-listed GyrB amino acid sequence from Escherichia coli.

In some cases, a member of an inducible dimer pair (e.g., a dimerizer-binding pair) is derived from DHFR (also known as dihydrofolate reductase, DHFRP1, and DYR). For example, a suitable dimerizer-binding pair member can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: 155)
MVGSLNCIVAVSQNMGIGKNGDLPWPPLRNEFRYFQRMTTTSSVEGKQN
LVIMGKKTWFSIPEKNRPLKGRINLVLSRELKEPPQGAHFLSRSLDDAL
KLTEQPELANKVDMVWIVGGSSVYKEAMNHPGHLKLFVTRIMQDFESDT
FFPEIDLEKYKLLPEYPGVLSDVQEEKGIKYKFEVYEKND.

In some cases, a member of an inducible dimer pair (e.g., a dimerizer-binding pair) is derived from the DmrB binding domain (i.e., DmrB homodimerization domain). For example, a suitable dimerizer-binding pair member can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: 156)
MASRGVQVETISPGDGRTFPKRGQTCVVHYTGMLEDGKKVDSSRDRNKP
FKFMLGKQEVIRGWEEGVAQMSVGQRAKLTISPDYAYGATGHPGIIPPH
ATLVFDVELLKLE.

In some cases, a member of an inducible dimer pair (e.g., a dimerizer-binding pair) is derived from a PYL protein (also known as abscisic acid receptor and as RCAR). For example a member of a subject dimerizer-binding pair can be derived from proteins such as those of Arabidopsis thaliana: PYR1, RCAR1 (PYL9), PYL1, PYL2, PYL3, PYL4, PYL5, PYL6, PYL7, PYL8 (RCAR3), PYL10, PYL11, PYL12, PYL13. For example, a suitable dimerizer-binding pair member can comprise an amino acid sequence having at least about 75%, at least about 80%, at least about 85%, at least about 90%, at least about 95%, at least about 98%, or 100% amino acid sequence identity to any of the following amino acid sequences:

PYL10:
(SEQ ID NO: 157)
MNGDETKKVESEYIKKHHRHELVESQCSSTLVKHIKAPLHLVWSIVRRFDEP
QKYKPFISRCVVQGKKLEVGSVREVDLKSGLPATKSTEVLEILDDNEHILGIRIVGGDH
RLKNYSSTISLHSETIDGKTGTLAIESFVVDVPEGNTKEETCFFVEALIQCNLNSLADV
TERLQAESMEKKI.
PYL11:
(SEQ ID NO: 158)
METSQKYHTCGSTLVQTIDAPLSLVWSILRRFDNPQAYKQFVKTCNLSSGDG
GEGSVREVTVVSGLPAEFSRERLDELDDESHVMMISIIGGDHRLVNYRSKTMAFVAA
DTEEKTVVVESYVVDVPEGNSEEETTSFADTIVGFNLKSLAKLSERVAHLKL
PYL12:
(SEQ ID NO: 159)
MKTSQEQHVCGSTVVQTINAPLPLVWSILRRFDNPKTFKHFVKTCKLRSGDG
GEGSVREVTVVSDLPASFSLERLDELDDESHVMVISIIGGDHRLVNYQSKTTVFVAAE
EEKTVVVESYVVDVPEGNTEEETTLFADTIVGCNLRSLAKLSEKMMELT.
PYL13:
(SEQ ID NO: 160)
MESSKQKRCRSSVVETIEAPLPLVWSILRSFDKPQAYQRFVKSCTMRSGGG
GGKGGEGKGSVRDVTLVSGFPADFSTERLEELDDESHVMVVSIIGGNHRLVNYKSK
TKVVASPEDMAKKTVVVESYVVDVPEGTSEEDTIFFVDNIIRYNLTSLAKLTKKMMK.
PYL1:
(SEQ ID NO: 161)
MANSESSSSPVNEEENSQRISTLHHQTMPSDLTQDEFTQLSQSIAEFHTYQLG
NGRCSSLLAQRIHAPPETVWSVVRRFDRPQIYKHFIKSCNVSEDFEMRVGCTRDVN
VISGLPANTSRERLDLLDDDRRVTGFSITGGEHRLRNYKSVTTVHRFEKEEEEERIWT
VVLESYVVDVPEGNSEEDTRLFADTVIRLNLQKLASITEAMNRNNNNNNSSQVR.
PYL2:
(SEQ ID NO: 162)
MSSSPAVKGLTDEEQKTLEPVIKTYHQFEPDPTTCTSLITQRIHAPASVVWPLI
RRFDNPERYKHFVKRCRLISGDGDVGSVREVTVISGLPASTSTERLEFVDDDHRVLS
FRVVGGEHRLKNYKSVTSVNEFLNQDSGKVYTVVLESYTVDIPEGNTEEDTKMFVDT
VVKLNLQKLGVAATSAPMHDDE.
PYL3:
(SEQ ID NO: 163)
MNLAPIHDPSSSSTTTTSSSTPYGLTKDEFSTLDSIIRTHHTFPRSPNTCTSLIA
HRVDAPAHAIWRFVRDFANPNKYKHFIKSCTIRVNGNGIKEIKVGTIREVSVVSGLPA
STSVEILEVLDEEKRILSFRVLGGEHRLNNYRSVTSVNEFVVLEKDKKKRVYSVVLES
YIVDIPQGNTEEDTRMFVDTVVKSNLQNLAVISTASPT.
PYL4:
(SEQ ID NO: 164)
MLAVHRPSSAVSDGDSVQIPMMIASFQKRFPSLSRDSTAARFHTHEVGPNQC
CSAVIQEISAPISTVWSVVRRFDNPQAYKHFLKSCSVIGGDGDNVGSLRQVHVVSGL
PAASSTERLDILDDERHVISFSVVGGDHRLSNYRSVTTLHPSPISGTVVVESYVVDVP
PGNTKEETCDFVDVIVRCNLQSLAKIAENTAAESKKKMSL.
PYL5:
(SEQ ID NO: 165)
MRSPVQLQHGSDATNGFHTLQPHDQTDGPIKRVCLTRGMHVPEHVAMHHTH
DVGPDQCCSSVVQMIHAPPESVWALVRRFDNPKVYKNFIRQCRIVQGDGLHVGDLR
EVMVVSGLPAVSSTERLEILDEERHVISFSVVGGDHRLKNYRSVTTLHASDDEGTVV
VESYIVDVPPGNTEEETLSFVDTIVRCNLQSLARSTNRQ.
PYL6:
(SEQ ID NO: 166)
MPTSIQFQRSSTAAEAANATVRNYPHHHQKQVQKVSLTRGMADVPEHVELSH
THVVGPSQCFSVVVQDVEAPVSTVWSILSRFEHPQAYKHFVKSCHVVIGDGREVGS
VREVRVVSGLPAAFSLERLEIMDDDRHVISFSVVGGDHRLMNYKSVTTVHESEEDSD
GKKRTRVVESYVVDVPAGNDKEETCSFADTIVRCNLQSLAKLAENTSKFS.
PYL7:
(SEQ ID NO: 167)
MEMIGGDDTDTEMYGALVTAQSLRLRHLHHCRENQCTSVLVKYIQAPVHLVW
SLVRRFDQPQKYKPFISRCTVNGDPEIGCLREVNVKSGLPATTSTERLEQLDDEEHIL
GINIIGGDHRLKNYSSILTVHPEMIDGRSGTMVMESFVVDVPQGNTKDDTCYFVESLI
KCNLKSLACVSERLAAQDITNSIATFCNASNGYREKNHTETNL.
PYL8:
(SEQ ID NO: 168)
MEANGIENLTNPNQEREFIRRHHKHELVDNQCSSTLVKHINAPVHIVWSLVRR
FDQPQKYKPFISRCVVKGNMEIGTVREVDVKSGLPATRSTERLELLDDNEHILSIRIVG
GDHRLKNYSSIISLHPETIEGRIGTLVIESFVVDVPEGNTKDETCYFVEALIKCNLKSLA
DISERLAVQDTTESRV.
PYL9:
(SEQ ID NO: 169)
MMDGVEGGTAMYGGLETVQYVRTHHQHLCRENQCTSALVKHIKAPLHLVWS
LVRRFDQPQKYKPFVSRCTVIGDPEIGSLREVNVKSGLPATTSTERLELLDDEEHILGI
KIIGGDHRLKNYSSILTVHPEIIEGRAGTMVIESFVVDVPQGNTKDETCYFVEALIRCNL
KSLADVSERLASQDITQ.
PYR1:
(SEQ ID NO: 170)
MPSELTPEERSELKNSIAEFHTYQLDPGSCSSLHAQRIHAPPELVWSIVRRFD
KPQTYKHFIKSCSVEQNFEMRVGCTRDVIVISGLPANTSTERLDILDDERRVTGFSIIG
GEHRLTNYKSVTTVHRFEKENRIWTVVLESYVVDMPEGNSEDDTRMFADTVVKLNL
QKLATVAEAMARNSGDGSGSQVT.

In some cases, a member of an inducible dimer pair (e.g., a dimerizer-binding pair) is derived from an ABI protein (also known as Abscisic Acid-Insensitive). For example a member of a subject dimerizer-binding pair can be derived from proteins such as those of Arabidopsis thaliana: ABI1 (Also known as ABSCISIC ACID-INSENSITIVE 1, Protein phosphatase 20 56, AtPP2C56, P2C56, and PP2C ABI1) and/or ABI2 (also known as P2C77, Protein phosphatase 2C 77, AtPP2C77, ABSCISIC ACID-INSENSITIVE 2, Protein phosphatase 2C ABI2, and PP2C ABI2). For example, a suitable dimerizer-binding pair member can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of any of the following amino acid sequences:

ABI1:
(SEQ ID NO: 171)
MEEVSPAIAGPFRPFSETQMDFTGIRLGKGYCNNQYSNQDSENGDLMVSLPE
TSSCSVSGSHGSESRKVLISRINSPNLNMKESAAADIVVVDISAGDEINGSDITSEKKM
ISRTESRSLFEFKSVPLYGFTSICGRRPEMEDAVSTIPRFLQSSSGSMLDGRFDPQS
AAHFFGVYDGHGGSQVANYCRERMHLALAEEIAKEMLCDGDTWLEKWKKALFNSF
LRVDSEIESVAPETVGSTSVVAVVFPSHIFVANCGDSRAVLCRGKTALPLSVDHKPD
REDEAARIEAAGGKVIQWNGARVFGVLAMSRSIGDRYLKPSIIPDPEVTAVKRVKED
DCLILASDGVWDVMTDEEACEMARKRILLWHKKNAVAGDASLLADERRKEGKDPAA
MSAAEYLSKLAIQRGSKDNISVVVVDLKPRRKLKSKPLN.
ABI2:
(SEQ ID NO: 172)
MDEVSPAVAVPFRPFTDPHAGLRGYCNGESRVTLPESSCSGDGAMKDSSFEI
NTRQDSLTSSSSAMAGVDISAGDEINGSDEFDPRSMNQSEKKVLSRTESRSLFEFK
CVPLYGVTSICGRRPEMEDSVSTIPRFLQVSSSSLLDGRVTNGFNPHLSAHFFGVYD
GHGGSQVANYCRERMHLALTEEIVKEKPEFCDGDTWQEKWKKALFNSFMRVDSEI
ETVAHAPETVGSTSVVAVVFPTHIFVANCGDSRAVLCRGKTPLALSVDHKPDRDDEA
ARIEAAGGKVIRWNGARVFGVLAMSRSIGDRYLKPSVIPDPEVTSVRRVKEDDCLILA
SDGLWDVMTNEEVCDLARKRILLWHKKNAMAGEALLPAEKRGEGKDPAAMSAAEY
LSKMALQKGSKDNISVVVVDLKGIRKFKSKSLN.

In some cases, a member of an inducible dimer pair (e.g., a dimerizer-binding pair) is derived from a Cry2 protein (also known as cryptochrome 2). For example a member of a subject dimer (e.g., a dimerizer-binding pair) can be derived from Cry2 proteins from any organism (e.g., a plant) such as, but not limited to, those of Arabidopsis thaliana. For example, a suitable dimerizer-binding pair member can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of any of the following amino acid sequences:

Cry2 (Arabidopsis thaliana):

(SEQ ID NO: 173)
MKMDKKTIVWFRRDLRIEDNPALAAAAHEGSVFPVFIWCPEEEGQFYPGRASRWW
MKQSLAHLSQSLKALGSDLTLIKTHNTISAILDCIRVTGATKVVFNHLYDPVSLVRDHT
VKEKLVERGISVQSYNGDLLYEPWEIYCEKGKPFTSFNSYWKKCLDMSIESVMLPPP
WRLMPITAAAEAIWACSIEELGLENEAEKPSNALLTRAWSPGWSNADKLLNEFIEKQL
IDYAKNSKKVVGNSTSLLSPYLHFGEISVRHVFQCARMKQIIWARDKNSEGEESADLF
LRGIGLREYSRYICFNFPFTHEQSLLSHLRFFPWDADVDKFKAWRQGRTGYPLVDA
GMRELWATGWMHNRIRVIVSSFAVKFLLLPWKWGMKYFWDTLLDADLECDILGWQ
YISGSIPDGHELDRLDNPALQGAKYDPEGEYIRQWLPELARLPTEWIHHPWDAPLTV
LKASGVELGTNYAKPIVDIDTARELLAKAISRTREAQIMIGAAPDEIVADSFEALGANTI
KEPGLCPSVSSNDQQVPSAVRYNGSKRVKPEEEEERDMKKSRGFDERELFSTAES
SSSSSVFFVSQSCSLASEGKNLEGIQDSSDQITTSLGKNGCK.

In some cases, a member of an inducible dimer pair (e.g., a dimerizer-binding pair) is derived from the CIB1 Arabidopsis thaliana protein (also known as transcription factor bHLH63). For example, a suitable dimer (e.g., a dimerizer-binding pair) member can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of the following amino acid sequence: MNGAIGGDLLLNFPDMSVLERQRAHLKYLNPTFDSPLAGFFADSSMITGG EMDSYLSTAGLNLPMMYGETTVEGDSRLSISPETTLGTGNFKKRKFDTETKDCNEKK KKMTMNRDDLVEEGEEEKSKITEQNNGSTKSIKKMKHKAKKEENNFSNDSSKVTKE LEKTDYIHVRARRGQATDSHSIAERVRREKISERMKFLQDLVPGCDKITGKAGMLDEI INYVQSLQRQIEFLSMKLAIVNPRPDFDMDDIFAKEVASTPMTVVPSPEMVLSGYSHE MVHSGYSSEMVNSGYLHVNPMQQVNTSSDPLSCFNNGEAPSMWDSHVQNLYGNL GV (SEQ ID NO: 174).

In some cases, a member of an inducible dimer pair (e.g., a dimerizer-binding pair) is derived from the GAI Arabidopsis thaliana protein (also known as Gibberellic Acid Insensitive, and DELLA protein GAI). For example, a suitable dimerizer-binding pair member can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of the following amino acid sequence:

(SEQ ID NO: 175)
MKRDHHHHHHQDKKTMMMNEEDDGNGMDELLAVLGYKVRSSEMADVAQKLEQLE
VMMSNVQEDDLSQLATETVHYNPAELYTWLDSMLTDLNPPSSNAEYDLKAIPGDAIL
NQFAIDSASSSNQGGGGDTYTTNKRLKCSNGVVETTTATAESTRHVVLVDSQENGV
RLVHALLACAEAVQKENLTVAEALVKQIGFLAVSQIGAMRKVATYFAEALARRIYRLS
PSQSPIDHSLSDTLQMHFYETCPYLKFAHFTANQAILEAFQGKKRVHVIDFSMSQGL
QWPALMQALALRPGGPPVFRLTGIGPPAPDNFDYLHEVGCKLAHLAEAIHVEFEYRG
FVANTLADLDASMLELRPSEIESVAVNSVFELHKLLGRPGAIDKVLGVVNQIKPEIFTV
VEQESNHNSPIFLDRFTESLHYYSTLFDSLEGVPSGQDKVMSEVYLGKQICNVVACD
GPDRVERHETLSQWRNRFGSAGFAAAHIGSNAFKQASMLLALFNGGEGYRVEESD
GCLMLGWHTRPLIATSAWKLSTN.

In some cases, a member of an inducible dimer pair (e.g., a dimerizer-binding pair) is derived from a GID1 Arabidopsis thaliana protein (also known as Gibberellin receptor GID1). For example, a suitable dimer member can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa of any of the following amino acid sequences:

GID1A:
(SEQ ID NO: 176)
MAASDEVNLIESRTVVPLNTWVLISNFKVAYNILRRPDGTFNRHLAEYLDRKV
TANANPVDGVFSFDVLIDRRINLLSRVYRPAYADQEQPPSILDLEKPVDGDIVPVILFF
HGGSFAHSSANSAIYDTLCRRLVGLCKCVVVSVNYRRAPENPYPCAYDDGWIALNW
VNSRSWLKSKKDSKVHIFLAGDSSGGNIAHNVALRAGESGIDVLGNILLNPMFGGNE
RTESEKSLDGKYFVTVRDRDWYWKAFLPEGEDREHPACNPFSPRGKSLEGVSFPK
SLVVVAGLDLIRDWQLAYAEGLKKAGQEVKLMHLEKATVGFYLLPNNNHFHNVMDEI
SAFVNAEC.
GID1B:
(SEQ ID NO: 177)
MAGGNEVNLNECKRIVPLNTWVLISNFKLAYKVLRRPDGSFNRDLAEFLDRK
VPANSFPLDGVFSFDHVDSTTNLLTRIYQPASLLHQTRHGTLELTKPLSTTEIVPVLIFF
HGGSFTHSSANSAIYDTFCRRLVTICGVVVVSVDYRRSPEHRYPCAYDDGWNALNW
VKSRVWLQSGKDSNVYVYLAGDSSGGNIAHNVAVRATNEGVKVLGNILLHPMFGGQ
ERTQSEKTLDGKYFVTIQDRDWYWRAYLPEGEDRDHPACNPFGPRGQSLKGVNFP
KSLVVVAGLDLVQDWQLAYVDGLKKTGLEVNLLYLKQATIGFYFLPNNDHFHCLMEE
LNKFVHSIEDSQSKSSPVLLTP.
GID1C:
(SEQ ID NO: 178)
MAGSEEVNLIESKTVVPLNTWVLISNFKLAYNLLRRPDGTFNRHLAEFLDRKV
PANANPVNGVFSFDVIIDRQTNLLSRVYRPADAGTSPSITDLQNPVDGEIVPVIVFFHG
GSFAHSSANSAIYDTLCRRLVGLCGAVVVSVNYRRAPENRYPCAYDDGWAVLKWV
NSSSWLRSKKDSKVRIFLAGDSSGGNIVHNVAVRAVESRIDVLGNILLNPMFGGTER
TESEKRLDGKYFVTVRDRDWYWRAFLPEGEDREHPACSPFGPRSKSLEGLSFPKSL
VVVAGLDLIQDWQLKYAEGLKKAGQEVKLLYLEQATIGFYLLPNNNHFHTVMDEIAAF
VNAECQ.

### Dimerizers

Various dimerizers (“dimerizing agents”) will be known to one of ordinary skill in the art and any convenient dimerizing agent can be used. Examples of dimerizing agents that can provide for dimerization of a first member of a dimerizer-binding pair and a second member of a dimerizer-binding pair include, but are not limited to (where the dimerizer is in parentheses following the dimerizer-binding pair):
a) FKBP1A and FKBP1A (rapamycin);
b) FKBP1A and CnA (rapamycin);
c) FKBP1A and cyclophilin (rapamycin);
d) FKBP1A and FRG (rapamycin);
e) GyrB and GyrB (coumermycin);
f) DHFR and DHFR (methotrexate);
g) DmrB and DmrB (AP20187);
h) PYL and ABI (abscisic acid);
i) Cry2 and CIB1 (blue light); and
j) GAI and GID1 (gibberellin).

As noted above, rapamycin can serve as a dimerizer. Alternatively, a rapamycin derivative or analog can be used. See, e.g., WO96/41865; WO 99/36553; WO 01/14387; and Ye et al (1999) Science 283:88-91. For example, analogs, homologs, derivatives and other compounds related structurally to rapamycin (“rapalogs”) include, among others, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Additional information is presented in, e.g., U.S. Pat. Nos. 5,525,610; 5,310,903 5,362,718; and 5,527,907. Selective epimerization of the C-28 hydroxyl group has been described; see, e.g., WO 01/14387. Additional synthetic dimerizing agents suitable for use as an alternative to rapamycin include those described in U.S. Patent Publication No. 2012/0130076.

Rapamycin has the structure:

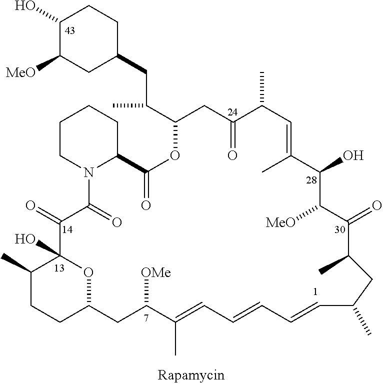

Suitable rapalogs include, e.g.,

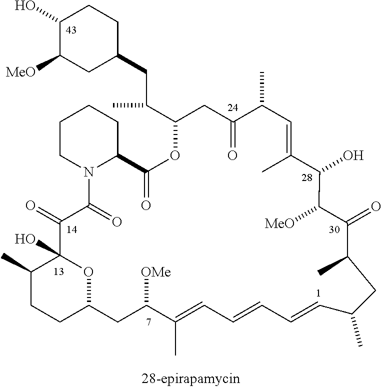

Also suitable as a rapalog is a compound of the formula:

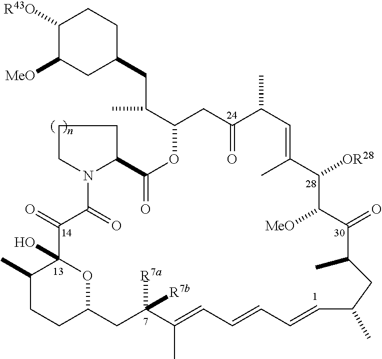

where n is 1 or 2; R²⁸ and R⁴³ are independently H, or a substituted or unsubstituted aliphatic or acyl moiety; one of R^{7a} and R^{7b} is H and the other is halo, R^{A}, OR^{A}, SRA, —OC(O)R^{A}, —OC(O)NR^{A}R^{B}, —NR^{A}R^{B}, —NR^{B}C(OR)R^{A}, NR^{B}C(O)OR^{A}, —NR^{B}SO₂R^{A}, or NR^{B}SO₂NR^{A}R^{B′}; or R^{7a} and R^{7b}, taken together, are H in the tetraene moiety:

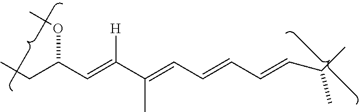

where R^{A} is H or a substituted or unsubstituted aliphatic, heteroaliphatic, aryl, or heteroaryl moiety and where R^{B} and R^{B′} are independently H, OH, or a substituted or unsubstituted aliphatic, heteroaliphatic, aryl, or heteroaryl moiety.

As noted above, coumermycin can serve as a dimerizing agent. Alternatively, a coumermycin analog can be used. See, e.g., Farrar et al. (1996) Nature 383:178-181; and U.S. Pat. No. 6,916,846.

As noted above, in some cases, the dimerizing agent is methotrexate, e.g., a non-cytotoxic, homo-bifunctional methotrexate dimer. See, e.g., U.S. Pat. No. 8,236,925

### Linkers

In some cases, a subject EvolvR polypeptide comprises a linker, e.g., positioned between the error-prone DNA polymerase and the RNA-guided endonuclease (e.g., CRISPR-Cas effector protein such as Slug-nCas9), positioned between the first member of a dimerizing pair and the RNA-guided endonuclease (e.g., CRISPR-Cas effector protein such as Slug-nCas9), positioned between the second member of the dimerizing pair and the error-prone DNA polymerase. A linker can be used and positioned as is convenient. For example, in some cases a linker is positioned between a heterologous protein (e.g., NLS, NES, protein tag, etc.) and the RNA-guided endonuclease (e.g., CRISPR-Cas effector protein such as Slug-nCas9), and/or is positioned between a heterologous protein (e.g., NLS, NES, protein tag, etc.) and the error-prone DNA polymerase. A subject EvolvR polypeptide can include any convenient number of linkers, e.g., one or more, two or more, three or more, four or more, five or more, six or more, 1, 2, 3, 4, 5, 6, etc.as is convenient.

For example, in some embodiments, a subject EvolvR polypeptide can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)n, glycine-serine polymers (including, for example, (GS)n, (GSGGS)n (SEQ ID NO:108), (GGSGGS)n (SEQ ID NO:109), and (GGGGS)n (SEQ ID NO:110), where n is an integer of at least one), glycine-alanine polymers, alanine-serine polymers. Exemplary linkers can comprise amino acid sequences including, but not limited to, GGSG (SEQ ID NO: 111), GGSGG (SEQ ID NO:112), GSGSG (SEQ ID NO:113), GSGGG (SEQ ID NO: 114), GGGSG (SEQ ID NO:115), GSSSG (SEQ ID NO:116), and the like. The ordinarily skilled artisan will recognize that design of a peptide conjugated to any desired element can include linkers that are all or partially flexible, such that the linker can include a flexible linker as well as one or more portions that confer less flexible structure.

### Localization Signals

In some cases, a heterologous polypeptide (a fusion partner) provides for subcellular localization, i.e., the heterologous polypeptide contains a subcellular localization sequence (e.g., a nuclear localization signal (NLS) to localize the EvolvR polypeptide to the nucleus, a sequence to keep the EvolvR polypeptide out of the nucleus, e.g., a nuclear export sequence (NES), a sequence to keep the EvolvR polypeptide retained in the cytoplasm, a mitochondrial localization signal for targeting to the mitochondria, a chloroplast localization signal for targeting to a chloroplast, an ER retention signal, and the like). In some cases, a subject EvolvR polypeptide does not include an NLS so that the protein is not targeted to the nucleus (e.g., in some cases in which it does include an NES) (which can be advantageous, e.g., when the target nucleic acid is present in the cytoplasm). When a dimerization pair (i.e., dimerizing pair) is used (e.g., where a nickase CRISPR-Cas effector polypeptide is fused to a first member of a dimerizing pair and an error-prone DNA polymerase is fused to a second member of the dimerizing pair), each fusion protein can be fused to a heterologous polypeptide such as a localization signal, e.g., each can be fused to one or more NLSs or fused to one or more NESs.

In some cases, a EvolvR polypeptide includes (is fused to) a nuclear localization signal (NLS) (e.g., in some cases 2 or more, 3 or more, 4 or more, or 5 or more NLSs). Thus, in some cases, a EvolvR polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at or near (e.g., within 50 amino acids of) the N-terminus and an NLS is positioned at or near (e.g., within 50 amino acids of) the C-terminus.

In some cases, a EvolvR polypeptide includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a EvolvR polypeptide includes (is fused to) between 2 and 5 NLSs (e.g., 2-4, or 2-3 NLSs).

Non-limiting examples of NLSs include an NLS sequence derived from: the NLS of the SV40 virus large T-antigen, having the amino acid sequence PKKKRKV (SEQ ID NO: 91); the NLS from nucleoplasmin (e.g., the nucleoplasmin bipartite NLS with the sequence KRPAATKKAGQAKKKK (SEQ ID NO:92)); the c-myc NLS having the amino acid sequence PAAKRVKLD (SEQ ID NO:93) or RQRRNELKRSP (SEQ ID NO: 94); the hRNPA1 M9 NLS having the sequence NQSSNFGPMKGGNFGGRSSGPYGGGGQYFAKPRNQGGY (SEQ ID NO:95); the sequence RMRIZFKNKGKDTAELRRRRVEVSVELRKAKKDEQILKRRNV (SEQ ID NO: 96) of the IBB domain from importin-alpha; the sequences VSRKRPRP (SEQ ID NO: 97) and PPKKARED (SEQ ID NO:98) of the myoma T protein; the sequence PQPKKKPL (SEQ ID NO:99) of human p53; the sequence SALIKKKKKMAP (SEQ ID NO: 100) of mouse c-abl IV; the sequences DRLRR (SEQ ID NO:101) and PKQKKRK (SEQ ID NO:102) of the influenza virus NS1; the sequence RKLKKKIKKL (SEQ ID NO: 103) of the Hepatitis virus delta antigen; the sequence REKKKFLKRR (SEQ ID NO: 104) of the mouse Mx1 protein; the sequence KRKGDEVDGVDEVAKKKSKK (SEQ ID NO:105) of the human poly (ADP-ribose) polymerase; and the sequence RKCLQAGMNLEARKTKK (SEQ ID NO:106) of the steroid hormone receptors (human) glucocorticoid. In some cases, an NLS comprises the amino acid sequence MDSLLMNRRKFLYQFKNVRWAKGRRETYLC (SEQ ID NO:107). In general, NLS (or multiple NLSs) are of sufficient strength to drive accumulation of the EvolvR polypeptide in a detectable amount in the nucleus of a eukaryotic cell. Detection of accumulation in the nucleus may be performed by any suitable technique. For example, a detectable marker may be fused to the EvolvR polypeptide such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, the NLS is located N-terminal of the RNA-guided endonuclease (e.g., Slug-nCas9) present in a subject EvolvR polypeptide. In some cases, the NLS is located between the DNA polymerase and the RNA-guided endonuclease (e.g., Slug-nCas9) present in a subject EvolvR polypeptide. In some cases, the NLS is located N-terminal of the DNA polymerase present in a subject EvolvR polypeptide. In some cases, the NLS is located C-terminal of the DNA polymerase present in a subject EvolvR polypeptide.

NESs are known in the art, and any NES can be used in a subject EvolvR polypeptide. See, e.g., Xu et al. (2012) Mol. Biol. Cell 23:3677; and Fung et al. (2017) eLife 6: e23961. Examples of NESs include, e.g., LPPLERLTL (SEQ ID NO:61); LALKLAGLDL (SEQ ID NO:62); MEELSQALASSFSV (SEQ ID NO:63); EAETVSAMALLSVG (SEQ ID NO:64); ELDELMASLSDFKF (SEQ ID NO:65); VDQLRLERLQI (SEQ ID NO:66); IDLSGLTLQ (SEQ ID NO:67); LRALERLQID (SEQ ID NO: 68); LOKKLEELEL (SEQ ID NO:69); MQELSNILNL (SEQ ID NO:70); LCQAFSDVIL (SEQ ID NO:71); RTFDMHSLESSLIDIMR (SEQ ID NO:72); TNLEALQKKLEELELDE (SEQ ID NO:73); RSFEMTEFNQALEEIKG (SEQ ID NO:74); PLQLPPLERLTL (SEQ ID NO:75); NELALKLAGLDI (SEQ ID NO:76); ERFEMFRELNEALEL (SEQ ID NO:77); DHAEKVAEKLEALSV (SEQ ID NO:78); QLVEELLKIICAFQL (SEQ ID NO:79); TNLEALQKKLEELEL (SEQ ID NO:80); DVKEEMTSALATMRV (SEQ ID NO:81); STNGSLAAEFRHLQL (SEQ ID NO:82); PSVQELTEQIHRLLM (SEQ ID NO:83); MNFKELKDFLKELNI (SEQ ID NO:84); ENFEILMKLKESLEL (SEQ ID NO:85); FETVYELTKMCTIRM (SEQ ID NO:86); SGKASSSLGLQDFDL (SEQ ID NO:87); PKYSDIDVDGLCSEL (SEQ ID NO:88); and VDLACTPTDVRDVDI (SEQ ID NO:89). An NES can have a length of from 8 amino acids to 25 amino acids. In some cases, the NES includes the following amino acid sequence: LPPLERLTL (SEQ ID NO:61). In some cases, the NES includes the following amino acid sequence: LPPLERLTL (SEQ ID NO:61) and has a length of 9 amino acids.

In some cases, a EvolvR polypeptide comprises, in order from N-terminus to C-terminus: i) a CRISPR-Cas effector polypeptide (e.g., a Slug-nCas9); ii) one or more NESs; and iii) an error-prone DNA polymerase. In some cases, a EvolvR polypeptide comprises, in order from N-terminus to C-terminus: i) a CRISPR-Cas effector polypeptide (e.g., a Slug-nCas9); ii) an error-protein DNA polymerase; and iii) one or more NESs. In some cases, a EvolvR polypeptide comprises, in order from N-terminus to C-terminus: i) one or more NESs; ii) a CRISPR-Cas effector polypeptide (e.g., a Slug-nCas9); and iii) an error-prone DNA polymerase. In some cases, a EvolvR polypeptide comprises, in order from N-terminus to C-terminus: i) a first NES; ii) a CRISPR-Cas effector polypeptide (e.g., a Slug-nCas9); iii) an error-prone DNA polymerase; and iv) a second NES. In some cases, a EvolvR polypeptide comprises, in order from N-terminus to C-terminus: i) a first NES; ii) a CRISPR-Cas effector polypeptide (e.g., a Slug-nCas9); iii) a second NES; and iv) an error-prone DNA polymerase. A peptide linker can be interposed between any two polypeptides in a fusion protein, e.g.: i) between an NES and a CRISPR-Cas effector polypeptide (e.g., a Slug-nCas9); ii) between a first NES and a second NES; iii) between a CRISPR-Cas effector polypeptide (e.g., a Slug-nCas9) and an error-prone DNA polymerase; iv) between an error-prone DNA polymerase and an NES; and the like.

### Additional Polypeptides

In some cases, the heterologous polypeptide can provide a tag (e.g., the heterologous polypeptide can be a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6XHis tag; a hemagglutinin (HA) tag; a FLAG tag; a Myc tag; and the like).

In some cases, a CRISPR-Cas effector EvolvR polypeptide includes a “Protein Transduction Domain” or PTD (also known as a CPP-cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked at or near (e.g., within 50 amino acids of) the amino terminus of a EvolvR polypeptide. In some embodiments, a PTD is covalently linked linked at or near (e.g., within 50 amino acids of) the carboxyl terminus of a EvolvR polypeptide. In some cases, the PTD is inserted internally in a EvolvR polypeptide at a suitable insertion site. Examples of PTDs include but are not limited to a minimal undecapeptide protein transduction domain (corresponding to residues 47-57 of HIV-1 TAT comprising YGRKKRRQRRR; SEQ ID NO: 179); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); a Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 189); Transportan GWTLNSAGYLLGKINLKALAALAKKIL (SEQ ID NO: 190); KALAWEAKLAKALAKALAKHLAKALAKALKCEA (SEQ ID NO: 191); and RQIKIWFQNRRMKWKK (SEQ ID NO: 192). Exemplary PTDs include but are not limited to, YGRKKRRQRRR (SEQ ID NO: 179), RKKRRQRRR (SEQ ID NO: 180); an arginine homopolymer of from 3 arginine residues to 50 arginine residues; Exemplary PTD domain amino acid sequences include, but are not limited to, any of the following: YGRKKRRQRRR (SEQ ID NO: 179); RKKRRQRR (SEQ ID NO: 181); YARAAARQARA (SEQ ID NO: 182); THRLPRRRRRR (SEQ ID NO: 183); and GGRRARRRRRR (SEQ ID NO: 184). In some cases, the PTD is an activatable CPP (ACPP) (Aguilera et al. (2009) Integr Biol (Camb) June; 1 (5-6): 371-381). ACPPs comprise a polycationic CPP (e.g., Arg9 or “R⁹”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### DNA-Binding Polypeptides

In some cases, an EvolvR polypeptide of the present disclosure (i.e., a subject EvolvR polypeptide) comprises a DNA-binding polypeptide that increases the processivity of the DNA polymerase. Suitable DNA-binding polypeptides that increase the processivity of the DNA polymerase include, but are not limited to, an Sso7d polypeptide, a helix-hairpin-helix domain of topoisomerase I, a thioredoxin binding domain of a T7 DNA polymerase, or a thioredoxin binding domain of a T3 polymerase. A topoisomerase I is set forth as SEQ ID NO: 21.

Suitable Sso7d polypeptides comprise an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Sso7d amino acid sequence of SEQ ID NO: 20.

Suitable thioredoxin binding domains comprise an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the following amino acid sequence:

(SEQ ID NO: 47)
TETFGSWYQPKGGTEMFCHPRTGKPLPKYPRIKTPKVGGIFKKPKNKA
QREGREPCELDTREYVAGAPYTPVEHV.

### Nucleic Acids; Recombinant Expression Vectors

The present disclosure provides a nucleic acid (e.g., an mRNA, a plasmid, a viral vector, a minicircle DNA) comprising a nucleotide sequence encoding a subject EvolvR polypeptide. In some cases, e.g., when a dimerization pair is used, the EvolvR polypeptide is encoded with two separate nucleotide sequences. In some such cases, both are present on the same nucleic acid (e.g., same viral vector, plasmid, and the like), and in other such cases each sequence is present on a different nucleic acid (e.g., mRNA, viral vector, plasmid, and the like). As such, provided is a system comprising one or more nucleic acids encoding a subject EvolvR polypeptide. In some cases, a subject system includes one nucleic acid. In some cases, a subject system includes two nucleic acid acids.

In some cases, a nucleic acid comprising a nucleotide sequence encoding a subject EvolvR polypeptide is contained within one or more expression vectors (e.g., 1 expression vector, 2 expression vectors). Thus, the present disclosure provides one or more recombinant expression vectors comprising a nucleotide sequence encoding a subject EvolvR polypeptide (e.g., in some cases one single nucleotide sequence, in some cases two nucleotide sequences). In some cases, the nucleotide sequence encoding a subject EvolvR polypeptide is operably linked to a transcriptional control element (e.g., a promoter; an enhancer; etc.). If the EvolvR polypeptide is encoded by two different nucleotide sequences, they can be operably linked to the same promoter (e.g., different copies of the same promoter) or they can be operably linked to different promoters. In some cases, the transcriptional control element(s) is inducible. In some cases, the transcriptional control element(s) is constitutive. In some cases, the promoters are functional in eukaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process).

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Examples of constitutive plant promoters include the cauliflower mosaic virus (CaMV) 35S promoter, which confers constitutive, high-level expression in most plant tissues (see, e.g., Odell et al. (1985) Nature 313:810-812); the nopaline synthase promoter (An et al. (1988) Plant Physiol. 88:547-552); and the octopine synthase promoter (Fromm et al. (1989) Plant Cell 1:977-984).

A variety of plant gene promoters that regulate gene expression in response to environmental, hormonal, chemical, developmental signals, and in a tissue-active manner can be used for expression of a nucleotide sequence (e.g., a nucleotide sequence encoding a subject EvolvR polypeptide) in plants. The choice of a promoter can be determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11:651-662), root-specific promoters, such as those disclosed in U.S. Pat. Nos. 5,618,988, 5,837,848 and 5,905, 186, pollen-active promoters such as PTA29, PTA26 and PTA13 (U.S. Pat. No. 5,792,929), promoters active in vascular tissue (Ringli and Keller (1998) Plant Mol. Biol. 37:977-988), flower-specific (Kaiser et al. (1995) Plant Mol. Biol. 28:231-243), pollen (Baerson et al. (1994) Plant Mol. Biol. 26:1947-1959), carpels (Ohl et al. (1990) Plant Cell 2:837-848), pollen and ovules (Baerson et al. (1993) Plant Mol. Biol. 22:255-267), auxin-inducible promoters (such as that described in van der Kop et al. (1999) Plant Mol. Biol. 39:979-990 or Baumann et al. (1999) Plant Cell 11:323-334), cytokinin-inducible promoter (Guevara-Garcia (1998) Plant Mol. Biol. 38:743-753), promoters responsive to gibberellin (Shi et al. (1998) Plant Mol. Biol. 38:1053-1060, Willmott et al. (1998) 38:817-825) and the like. Additional promoters are those that elicit expression in response to heat (Ainley et al. (1993) Plant Mol. Biol. 22:13-23), light (e.g., the pea rbcS-3A promoter, Kuhlemeier et al. (1989) Plant Cell 1:471-478, and the maize rbcS promoter, Schaffner and Sheen (1991) Plant Cell 3:997-1012); wounding (e.g., wunl, Siebertz et al. (1989) Plant Cell 1:961-968); pathogens (such as the PR-1 promoter described in Buchel et al. (1999) Plant Mol. Biol. 40:387-396, and the PDF1.2 promoter described in Manners et al. (1998) Plant Mol. Biol. 38:1071-1080), and chemicals such as methyl jasmonate or salicylic acid (Gatz (1997) Annu. Rev. Plant Physiol. Plant Mol. Biol. 48:89-108). In addition, the timing of the expression can be controlled by using promoters such as those acting at senescence (Gan and Amasino (1995) Science 270:1986-1988); or late seed development (Odell et al. (1994) Plant Physiol. 106:447-458).

Non-limiting examples of eukaryotic promoters (promoters functional in a eukaryotic cell) include EF1a, those from cytomegalovirus (CMV) immediate early, herpes simplex virus (HSV) thymidine kinase, beta actin, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6xHis tag, hemagglutinin tag, fluorescent protein, etc.) that can be fused to a subject protein, thus resulting in a chimeric polypeptide.

Promoters can be derived from viruses and can therefore be referred to as viral promoters, or they can be derived from any organism, including prokaryotic or eukaryotic organisms. Promoters can be used to drive expression by any RNA polymerase (e.g., pol I, pol Il, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), beta actin, EF1-alpha, a rous sarcoma virus (RSV) promoter, and the like. Example Pol III promoters (e.g., for expressing a guide RNA) include, but are not necessarily limited to: a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), and the like.

In some cases, a nucleotide sequence encoding a guide RNA is operably linked to (under the control of) a promoter operable in a eukaryotic cell (e.g., a Pol III promoter such as a U6 promoter, an enhanced U6 promoter, an H1 promoter, and the like). In some cases, a nucleotide sequence encoding a subject is operably linked to a promoter operable in a prokaryotic cell such as a bacterial cell, e.g., E. coli. In some cases, a nucleotide sequence encoding a subject EvolvR polypeptide is operably linked to a promoter operable in a eukaryotic cell (e.g., a CMV promoter, a beta-actin promoter, an EF1a promoter, an estrogen receptor-regulated promoter, and the like).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (ATC)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, a nucleic acid comprising a nucleotide sequence encoding a subject EvolvR polypeptide is a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a subject EvolvR polypeptide is a recombinant lentivirus vector. In some cases, a nucleic acid comprising a nucleotide sequence encoding a subject EvolvR polypeptide is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303:209, Bevan (1984) Nucleic Acids Res. 12:8711-8721, Klee (1985) Bio/Technology 3:637-642, for dicotyledonous plants.

Alternatively, non-Ti vectors can be used to transfer a nucleic acid into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) Bio/Technology 9:957-962) and corn (Gordon-Kamm (1990) Plant Cell 2:603-618) can be produced. An immature embryo can also be a good target tissue for monocots for direct DNA delivery techniques by using the particle gun (Weeks et al. (1993) Plant Physiol. 10²:1077-1084; Vasil (1993) Bio/Technology 10:667-674; Wan and Lemeaux (1994) Plant Physiol. 104:37-48, and for Agrobacterium-mediated DNA transfer (Ishida et al. (1996) Nature Biotechnol. 14:745-750).

Methods of introducing a nucleic acid into a host cell are known in the art, and any convenient method can be used to introduce a nucleic acid (e.g., an expression construct) into a cell. Suitable methods include e.g., viral infection, transfection, lipofection, nucleofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, lipid nanoparticle mediated delivery, and the like.

Introducing the recombinant expression vector into cells can occur in vivo or can occur in any culture media and under any culture conditions that promote the survival of the cells. Introducing the recombinant expression vector into a target cell can be carried out in vivo or ex vivo or in vitro. Introducing the recombinant expression vector into a target cell can be carried out in vitro.

In some embodiments, a subject protein (e.g., deaminase fusion protein and/or CRISPR-Cas fusion protein) is provided to a cell as RNA (e.g., as mRNA that is translated into protein within a cell). In some embodiments, a guide RNA is provided as RNA. The RNA can be provided by direct chemical synthesis or may be transcribed in vitro from a DNA. Once synthesized, the RNA may be introduced into a cell by any of the well-known techniques for introducing nucleic acids into cells (e.g., microinjection, electroporation, transfection, lipid nanoparticle delivery, etc.).

Nucleic acids may be provided to the cells using well-developed transfection techniques; see, e.g. Angel and Yanik (2010) PLOS ONE 5 (7): e11756, and the commercially available TransMessenger® reagents from Qiagen, Stemfect™ RNA Transfection Kit from Stemgent, TransIT®-mRNA Transfection Kit from Mirus Bio LLC, nucleofection, and the like. See also Beumer et al. (2008) PNAS 105 (50): 19821-19826.

Vectors may be provided directly to a target host cell. In other words, the cells can be contacted with vectors comprising the subject nucleic acids such that the vectors are taken up by the cells. Methods for contacting cells with nucleic acid vectors that are plasmids include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Methods of introducing a nucleic acid into a host cell are known in the art, and any convenient method can be used to introduce a nucleic acid (e.g., an expression construct) into a cell. Suitable methods include e.g., viral infection, transfection, lipofection, nucleofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, lipid nanoparticle mediated delivery, and the like.

Introducing the recombinant expression vector into cells can occur in vivo or can occur in any culture media and under any culture conditions that promote the survival of the cells. Introducing the recombinant expression vector into a target cell can be carried out in vivo or ex vivo or in vitro. Introducing the recombinant expression vector into a target cell can be carried out in vitro.

In some embodiments, a subject protein is provided to a cell as RNA (e.g., as mRNA that is translated into protein within a cell). In some embodiments, a guide RNA is provided as RNA. The RNA can be provided by direct chemical synthesis or may be transcribed in vitro from a DNA. Once synthesized, the RNA may be introduced into a cell by any of the well-known techniques for introducing nucleic acids into cells (e.g., microinjection, electroporation, transfection, lipid nanoparticle delivery, etc.).

Additionally or alternatively, a protein of the present disclosure can be introduced as protein into a cell (e.g., in some cases Fused to a polypeptide permeant domain to promote uptake by the cell). In some cases, a subject a EvolvR poypeptide is introduced to a cell as an RNP (i.e., pre-complexed with a guide RNA). In some cases, a RNP is delivered using peptide-enabled RNP delivery for CRISPR engineering (PERC) (see, e.g., Foss et al., Nature Biomedical Engineering, 2023: p. 1-14). In some cases, a RNP is delivered using a nanoparticle formulation. In some cases, a RNP is delivered using a lipid nanoparticle formulation.

Nucleic acids may be provided to the cells using well-developed transfection techniques; see, e.g. Angel and Yanik (2010) PLOS ONE 5 (7): e11756, and the commercially available TransMessenger@ reagents from Qiagen, Stemfect™ RNA Transfection Kit from Stemgent, TransIT®-mRNA Transfection Kit from Mirus Bio LLC, nucleofection, and the like. See also Beumer et al. (2008) PNAS 105 (50): 19821-19826.

Vectors may be provided directly to a target host cell. In other words, the cells can be contacted with vectors comprising the subject nucleic acids such that the vectors are taken up by the cells. Methods for contacting cells with nucleic acid vectors that are plasmids include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

In some embodiments, a nucleotide sequence encoding a protein of the present disclosure (e.g., an EvolvR polypeptide) is codon optimized. This type of optimization can entail a mutation of a protein-coding nucleotide sequence to mimic the codon preferences of the intended host organism or cell while encoding the same protein (e.g., using commercially or freely available software). Thus, the codons can be changed, but the encoded protein remains unchanged. For example, if the intended target cell was a human cell, a human codon-optimized protein-encoding nucleotide sequence could be used. As another non-limiting example, if the intended host cell were a mouse cell, then a mouse codon-optimized protein-encoding nucleotide sequence could be generated. As another non-limiting example, if the intended host cell were a plant cell, then a plant codon-optimized protein-encoding nucleotide sequence could be generated. As another non-limiting example, if the intended host cell were an insect cell, then an insect codon-optimized protein-encoding nucleotide sequence could be generated. As another non-limiting example, if the intended host cell were a prokaryotic cell such as a bacterial cell (e.g., E. coli), then a bacterial (e.g., E. coli) codon-optimized protein-encoding nucleotide sequence could be generated. In some cases, the nucleotide sequence(s) is codon optimized for expression in a prokaryotic cell. In some cases, the nucleotide sequence(s) is codon optimized for expression in an E. coli cell. In some cases, the nucleotide sequence(s) is codon optimized for expression in a eukaryotic cell. In some cases, the nucleotide sequence(s) is codon optimized for expression in a mammalian cell.

### Host Cells and Target Cells

The present disclosure provides a cell comprising a subject EvolvR polypeptide. The present disclosure provides a cell comprising a system of the present disclosure. The present disclosure provides a cell comprising a nucleic acid (e.g., a recombinant expression vector) of the present disclosure.

Suitable host cells include, e.g. a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. Agardh, and the like; a fungal cell (e.g., a yeast cell); an animal cell; an invertebrate animal cell (e.g. fruit fly, cnidarian, echinoderm, nematode, etc.); a vertebrate animal cell (e.g., fish, amphibian, reptile, bird, mammal); a mammalian cell (e.g., a rodent, a human, non-human primate, equine, bovine, porcine, canine, feline, ungulate, etc.); and the like.

A suitable host cell can be a stem cell (e.g. an embryonic stem (ES) cell, an induced pluripotent stem (iPS) cell); a germ cell; a somatic cell, e.g. a fibroblast, a hematopoietic cell, a neuron, a oligodendrocyte, an astrocyte, a muscle cell, a bone cell, a hepatocyte, a pancreatic cell (e.g., an islet cell); an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages of the culture. For example, primary cultures include cultures that may have been passaged 0 times, 1 time, 2 times, 4 times, 5 times, 10 times, or 15 times, but not enough times go through the crisis stage. Primary cell lines can be maintained for fewer than 10 passages in vitro. Host cells are in some cases unicellular organisms, or are grown in culture.

If the cells are primary cells, they may be harvest from an organism (e.g., an individual) by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, e.g., from 5-25 mM. Convenient buffers include HEPES, phosphate buffers, lactate buffers, etc. The cells may be used immediately, or they may be stored, frozen, for long periods of time, being thawed and capable of being reused. In such cases, the cells can be frozen in 10% dimethyl sulfoxide (DMSO), 50% serum, 40% buffered medium, or some other such solution as is commonly used in the art to preserve cells at such freezing temperatures, and thawed in a manner as commonly known in the art for thawing frozen cultured cells.

In some cases, a subject genetically modified host cell is in vitro (e.g., a cell in culture). In some embodiments, a subject genetically modified host cell is in vivo. In some cases, a subject genetically modified host cell is ex vivo (e.g., a version of in vitro in which a freshly isolated, i.e., low passage, cell such as a primary cell is in culture). In some embodiments, a subject genetically modified host cell is a prokaryotic cell or is derived from a prokaryotic cell. In some embodiments, a subject genetically modified host cell is a bacterial cell or is derived from a bacterial cell. In some cases, a subject genetically modified host cell is an archaeal cell or is derived from an archaeal cell. In some embodiments, a subject genetically modified host cell is a eukaryotic cell or is derived from a eukaryotic cell. In some cases, a subject genetically modified host cell is a plant cell or is derived from a plant cell. In some cases, a subject genetically modified host cell is an animal cell or is derived from an animal cell. In some embodiments, a subject genetically modified host cell is an invertebrate cell or is derived from an invertebrate cell. In some cases, a subject genetically modified host cell is a vertebrate cell or is derived from a vertebrate cell. In some cases, a subject genetically modified host cell is a mammalian cell or is derived from a mammalian cell. In some cases, a subject genetically modified host cell is a rodent cell or is derived from a rodent cell. In cases embodiments, a subject genetically modified host cell is a human cell or is derived from a human cell.

The present disclosure provides a genetically modified plant cell, where the genetically modified plant cell is genetically modified with a nucleic acid (e.g., a recombinant expression vector) comprising a nucleotide sequence encoding a subject EvolvR polypeptide. In some cases, the plant cell is a cell of a monocot (a monocotyledon). In some cases, the plant cell is a cell of a dicot (a dicotyledon). A plant cell can be a cell of the xylem, the phloem, the cambium layer, a leaf, a root, etc.

Suitable plants include, e.g., soybean, wheat, corn, potato, cotton, rice, oilseed rape, sunflower, alfalfa, clover, sugarcane, turf, banana, blackberry, blueberry, strawberry, raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, watermelon, mint and other labiates, rosaceous fruits, and vegetable brassicas.

Plant protoplasts are also suitable for some applications. For example, a nucleic acid is introduced into plant tissues, cultured plant cells or plant protoplasts by standard methods including electroporation (Fromm et al. (1985) Proc. Natl. Acad. Sci. 82:5824-5828), infection by viral vectors such as cauliflower mosaic virus (CaMV) (Hohn et al. (1982) Molecular Biology of Plant Tumors Academic Press, New York, N.Y., pp. 549-560; U.S. Pat. No. 4,407,956), high velocity ballistic penetration by small particles with the nucleic acid either within the matrix of small beads or particles, or on the surface (Klein et al. (1987) Nature 327:70-73), use of pollen as vector (WO 85/01856), or use of Agrobacterium tumefaciens or A. rhizogenes carrying a T-DNA plasmid in which a nucleic acid encoding a subject EvolvR polypeptide is cloned. The T-DNA plasmid is transmitted to plant cells upon infection by Agrobacterium tumefaciens, and a portion is stably integrated into the plant genome (Horsch et al. (1984) Science 233:496-498; Fraley et al. (1983) Proc. Natl. Acad. Sci. 80:4803-4807).

The present disclosure further provides progeny of a subject genetically modified cell, where the progeny can comprise the same exogenous nucleic acid or polypeptide as the subject genetically modified cell from which it was derived. The present disclosure further provides a composition comprising a subject genetically modified host cell.

### Methods

A subject EvolvR polypeptide is useful for introducing mutations into a target region of a target nucleic acid; i.e., in some cases, a subject EvolvR polypeptide functions as a mutator. Thus, the present disclosure provides methods of modifying a target nucleic acid (e.g., a target DNA) (e.g., introducing mutations into a target region of a target nucleic acid). In some cases, a subject method is a method of mutagenizing a target nucleic, e.g., introducing mutations into a target nucleic acid.

In some embodiments, a subject method includes contacting a target nucleic acid (e.g., target DNA) with a system of the present disclosure (e.g., EvolvR polypeptide); i.e., the method comprises contacting the target nucleic acid with a complex (an RNP) that includes: (a) a subject EvolvR polypeptide; and b) a guide RNA. In some cases, the target nucleic acid is present in a cell. In some such cases, a subject method comprises introducing a system of the present disclosure into the cell.

As would be known to one of ordinary skill in the art, the guide RNA of the RNP could be introduced into the cell as an RNA molecule (e.g., unmodified or modified), or as a DNA molecule encoding the guide RNA. Similarly, the EvolvR polypeptide could be introduced into the cell as a protein(s), as RNA encoding the protein(s), or as DNA encoding the protein(s) [the “(s)” is used to denote that in some cases the EvolvR polypeptide is two distinct proteins, e.g., when a dimerizing pair is used]. Also, the guide RNA and the EvolvR polypeptide could be introduced into the cell as a preformed RNP complex.

Any of the cells discussed above in the context of host cells can be a target cell for a subject method. For example, in some cases, the cell is a prokaryotic cell. In some cases, the cell is a eukaryotic cell. In some cases, the cell is a plant cell. In some cases, the plant cell is a cell of a dicotyledonous plant. In some cases, the plant cell is a cell of a monocotyledonous plant. In some cases, the cell is a mammalian cell (e.g., human cell, non-human primate cell, mouse cell, rat cell, etc.). In some cases, the cell is in vitro. In some cases, the cell is in vivo. In some cases, the cell is ex vivo.

Methods of the present disclosure finds use in a variety of applications. Non-limiting examples of applications involving use of an EvolvR polypeptide of the present disclosure include: a) diversifying antibody-encoding genes; b) diversifying protein-coding nucleotide sequences; c) diversifying regulatory elements; d) optimizing antibody affinity; e) diversifying T cells; f) engineering T-cell activity; g) discovering disease-causing genotypes; h) engineering desirable traits into plants; I) mutagenizing viruses (e.g., large DNA viruses) in the cytoplasm of a eukaryotic cell.

In some cases, contacting a target nucleic acid with a complex comprising: a) a subject EvolvR polypeptide; and b) a guide RNA results in introduction of from 1 mutation to 10³ mutations within a target region of a target nucleic acid. For example, in some cases, contacting a target nucleic acid with a complex comprising: a) a subject EvolvR polypeptide; and b) a guide RNA results in introduction of from 1 mutation to 5 mutations, from 5 mutations to 10 mutations, from 10 mutations to 50 mutations, from 50 mutations to 10² mutations, from 10² mutations to 5×10² mutations, or from to 5×10² mutations to 10³ mutations within a target region of a target nucleic acid. As noted above, in some cases, the target region of a target nucleic acid is from 1 nucleotide (nt) to 10 nucleotides (nt), from 10 nt to 50 nt, from 50 nt to 100 nt, from 100 nt to 500 nt, from 500 nt to 10³ nt, from 10³ nt to 5×10³ nt, or from 5×10³ nt to 10⁴ nt from a nick in a target DNA introduced by the RNA-guided endonuclease.

Introducing mutations into a target region of a target nucleic acid provides for generation of a plurality of mutants, which can then be selected for a particular desired trait. Alternatively, an undesirable trait can be selected against. A desired trait can be selected for simultaneously with selecting against an undesired trait.

In some cases, a method of the present disclosure comprises: a) mutagenizing a target nucleic acid, generating a plurality of mutated nucleic acids; and b) applying a selection to the mutated nucleic acids. Applying a selection to the mutated nucleic acids can comprise: i) selecting a mutated nucleic acid(s) that confers a desirable trait (phenotype) on a genetically modified host cell comprising the mutated nucleic acid; or ii) selecting a mutated nucleic acid(s) that confers a desirable trait (phenotype) on a transgenic non-human organism that is genetically modified to comprise the mutated nucleic acid. Selection methods are well known in the art, and any known method can be applied.

For example, in some cases, a mutated nucleic acid may confer increased drought resistance to a plant; and the mutated nucleic acid is identified by subjecting a plurality of plants (or a plurality of plant seeds), each of which is genetically modified with a single member of the plurality of mutated nucleic acids, to drought conditions, and selecting plants that exhibit increased resistance to the drought conditions. Drought assays can be applied to identify a mutated nucleic acid that confers better plant survival after short-term, severe water deprivation. lon leakage can be measured in the context of a drought assay.

As another example, in some cases, a mutated nucleic acid may confer increased resistance of a plant to a pathogen (e.g., a fungus; an insect; etc.); and the mutated nucleic acid is identified by subjecting a plurality of plants (or a plurality of plant seeds), each of which is genetically modified with a single member of the plurality of mutated nucleic acids, to the pathogen, and selecting plants that exhibit increased resistance to the pathogen.

As another example, in some cases, a mutated nucleic acid may confer increased resistance of a plant to salt stress (e.g., high salt conditions; e.g., high NaCl concentrations); and the mutated nucleic acid is identified by subjecting a plurality of plants (or a plurality of plant seeds), each of which is genetically modified with a single member of the plurality of mutated nucleic acids, to high salt conditions, and selecting plants that exhibit increased resistance to the high salt conditions. Plants differ in their tolerance to NaCl depending on their stage of development; therefore seed germination, seedling vigor, and plant growth responses can be evaluated to determine resistance to high salt conditions.

As another example, in some cases, a mutated nucleic acid may confer increased resistance of a plant to freezing; and the mutated nucleic acid is identified by subjecting a plurality of plants (or a plurality of plant seeds), each of which is genetically modified with a single member of the plurality of mutated nucleic acids, to low temperature (e.g., freezing) conditions, and selecting plants that exhibit increased resistance to the low temperature conditions.

As another example, in some cases, a mutated nucleic acid may confer increased ability to germinate under high temperature conditions; and the mutated nucleic acid is identified by subjecting a plurality of plants (or a plurality of plant seeds), each of which is genetically modified with a single member of the plurality of mutated nucleic acids, to high temperature conditions, and selecting plants that exhibit increased resistance to the high temperature conditions. Parameters that can be tested include seed germination, seedling vigor, and plant growth.

As another example, in some cases, a mutated nucleic acid may confer increased resistance to hyperosmotic stress; and the mutated nucleic acid is identified by subjecting a plurality of plants (or a plurality of plant seeds), each of which is genetically modified with a single member of the plurality of mutated nucleic acids, to hyperosmotic stress conditions, and selecting plants that exhibit increased resistance to the hyperosmotic stress conditions. Plants that are resistant to hyperosmotic stress may be more tolerant to drought or freezing.

Sugar sensing assays can be conducted to identify mutated nucleic acids that provide for sugar sensing by germinating seeds on high concentrations of sucrose and glucose and looking for degrees of hypocotyl elongation. The germination assay on mannitol controls for responses related to hyperosmotic stress. Sugars are key regulatory molecules that affect diverse processes in higher plants including germination, growth, flowering, senescence, sugar metabolism and photosynthesis. Sucrose is the major transport form of photosynthate and its flux through cells has been shown to affect gene expression and alter storage compound accumulation in seeds (source-sink relationships). Glucose-specific hexose-sensing has also been described in plants and is implicated in cell division and repression of “famine” genes (photosynthetic or glyoxylate cycles).

Crop productivity is in part limited by its rate of CO₂ fixation by the RuBisCo enzyme. In some cases, all RuBisCo catalytic subunits are simultaneously targeted within cyanobacteria using a system of the present disclosure; growing this microbe under high temperature and/or low CO₂ conditions will enrich for RuBisco variants with improved catalytic efficiency and specificity.

### Cytoplasmic Viral Nucleic Acid

In some embodiments, the target nucleic acid is a viral nucleic acid. In some cases, the viral nucleic acid is in the cytoplasm of a eukaryotic cell. See, e.g., international patent application publication WO2024011173, which is incorporated herein by reference in its entirety, e.g., for all disclosure related to targeting a nucleic acid in the cytoplasm of a eukaryotic cell.

As such, in some cases, a subject EvolvR polypeptide is used to modify a target viral nucleic acid in the cytoplasm of a eukaryotic cell. In some such cases, the EvolvR polypeptide includes (e.g., is fused to) one or more nuclear export signals (NESs). Examples of NESs are provided as SEQ ID NOs: 61-89 (see above). Thus, in some cases a subject method includes contacting a target viral nucleic acid in the cytoplasm of a eukaryotic cell with a ribonucleoprotein complex (RNP) that includes a guide RNA and a subject EvolvR polypeptide [one that includes (e.g., is fused to) one or more nuclear export signals (NESs)]. In some cases, the EvolvR polypeptide does not include a nuclear localization signal (NLS).

Viral nucleic acids that can be modified using a method of the present disclosure are referred to as “target viral nucleic acids.” A suitable target viral nucleic acid is a double-stranded DNA virus that has a genome length of from about 50 kilo base pairs (kbp) to about 1.2 mega base pairs (mbp), where at least part of the replication cycle of the double-stranded DNA virus occurs in the cytoplasm of the cell. Such viruses are sometimes referred to as “nucleocytoplasmic large DNA viruses” or “NCLDVs”. In some cases, a suitable target viral nucleic acid is a double-stranded DNA virus that has a genome length of from about 50 kbp to 150 kbp, from about 150 kbp to about 500 kbp, from about 500 kbp to about 1000 kbp, or from about 1000 kbp to about 1.2 mbp.

NCLDVs encompass multiple viral families, including Poxviridae, Asfaviridae, Iridoviridae, Ascoviridae, Phycodnaviridae, Marseilleviridae, Pithoviridae, Mimiviridae, Pandoraviridae, Mininucleoviridae, Molliviruses, and Faustoviruses.

In some cases, the target viral nucleic acid is a member of Poxviridae. Poxiviridae includes the genuses Avipoxvirus, Capripoxvirus, Centapoxvirus, Cervidpoxvirus, Crocodylidpoxvirus, Leporipoxvirus, Macroopoxvirus, Molluscipoxvirus, Mustelpoxvirus, Orthopoxivirus, Oryzopoxvirus, Parapoxvirus, Pteropopoxvirus, Scieuripoxvirus, Suipoxvirus, Vespertilionpoxvirus, Yatapoxvirus, Alphaentemopoxvirus, Betaentemopoxvirus, Deltaentomopoxvirus, Diachasmimorphaentemopoxvirus, and Gammaentomopoxvirus. The Orthopoxvirus genus includes vaccinia virus, cowpox virus, monkeypox virus, and rabbitpox virus. In some cases, the target viral nucleic acid is a nucleic acid of a Myxoma virus, Squirrel Fibroma Virus, or an Ectromelia virus. In some cases, the target viral nucleic acid is a vaccinia virus. In some cases, the target viral nucleic acid is a vaccinia virus of any one of the following vaccinia virus strains: 1) Western Reserve; 2) Wyeth; 3) New York City Board of Health (NYCBH); 4) Paris; 5) Acambis 2000; 6) Bern; 7) Ankara; 8) IHD-J; 9) Copenhagen (Cop); 10) Temple of Heaven; 11) Dairen; 12) Lister; 13) Tian Tan; 14) Modified Vaccinia Ankara (MVA);, 15) Lister clone 16m8 (LC16m8); and 16) Dairen I (DIs). In some cases, the target viral nucleic acid is a nucleic acid of a chimeric poxvirus strain or a recombinant viral strain encoding heterologous DNA.

In some cases, a target nucleotide sequence in a target viral nucleic acid is a coding sequence, e.g., the target nucleotide sequence encodes a polypeptide and/or an RNA. A target nucleotide sequence in a target viral nucleic acid can be a nucleotide sequence in a coding sequence that encodes a polypeptide such as a polypeptide that is involved in dissemination of the virus, an antigenic polypeptide (e.g., a polypeptide in the viral capsid), a polypeptide that provides for oncolytic activity, a polypeptide that functions in release of a virus from a cell, a polypeptide that provides for infectivity of a virus, a polypeptide that alters the cell or host specificity of a virus, a polypeptide that alters the entry mechanism of a virus, a polypeptide that alters the intracellular trafficking of a virus, a polypeptide that alters the actin-based propulsion of a virus in intra- or extra-cellular environments, a polypeptide that alters microtubule association or trafficking of a virus, a polypeptide involved in the formation and maturation of intracellular mature virion (IMV), a polypeptide involved in the formation of cell-associated enveloped virion (CEV), a polypeptide involved in the formation of extracellular enveloped virus (EEV), a virally encoded polypeptide that functions in antagonizing the host innate or adaptive antiviral immune response, and the like.

Any vaccinia virus gene can be a target nucleic acid. For example, vaccinia virus coding regions that may be of interest as target nucleotides include a vaccinia virus gene selected from F13L, A36R, A34R, A53R, B5R, B7R, B13R, B15R, B22R, B28R, B29R, A33R, B8R, B18R, SPI-1, SPI-2, B15R, CUR, VGF, E3L, K2L, K3L, A41L, K7R, vC12L, vCKBP, and N1L. For example, A34R is a vaccinia virus glycoprotein required for cellular release and infectivity of EEV. B5R, F13L, A36R, A34R, and A33R are examples of EEV-specific membrane proteins.

In some cases, a method of the present disclosure provides for introduction into a target viral nucleic acid one or more mutations, thereby generating a variant virus, where the variant virus exhibits increased oncolytic activity compared to the unmutated virus (i.e., a control virus that does not include the one or more mutations but is otherwise identical to the variant virus). In some cases, a method of the present disclosure provides for introduction into a target viral nucleic acid one or more mutations, thereby generating a variant virus, where the variant virus exhibits oncolytic activity that is at least 10%, at least 25%, at least 50%, at least 100% (or 2-fold), at least 5-fold, at least 10-fold, or more than 10-fold, higher than the oncolytic activity of the unmutated virus (i.e., a control virus that does not include the one or more mutations but is otherwise identical to the variant virus).

In some cases, a method of the present disclosure provides for introduction into a target viral nucleic acid one or more mutations, thereby generating a variant virus, where the variant virus exhibits increased production of EEV compared to the unmutated virus (i.e., a control virus that does not include the one or more mutations but is otherwise identical to the variant virus). In some cases, a method of the present disclosure provides for introduction into a target viral nucleic acid one or more mutations, thereby generating a variant virus, where the variant virus exhibits at least 10%, at least 25%, at least 50%, at least 100% (or 2-fold), at least 5-fold, at least 10-fold, or more than 10-fold, greater production of EEV, compared to the unmutated virus (i.e., a control virus that does not include the one or more mutations but is otherwise identical to the variant virus).

In some cases, a method of the present disclosure provides for introduction of one or more mutations into a target viral nucleic acid, thereby generating a variant virus, where the variant virus exhibits reduced neutralization by neutralizing antibodies in a mammalian host (e.g., a human). In some cases, a method of the present disclosure provides for introduction of one or more mutations into a target viral nucleic acid, thereby generating a variant virus, where the variant virus exhibits one or more of: 1) increased virion production in target cells; 2) increased CEV formation; 3) increased EEV formation; 4) modified mechanism of cell entry conferring altered cell tropism; 5) increased intracellular trafficking; 6) increased kinetics of viral replication, maturation, and egress; and 6) improved dissemination among metastasized tumor cells within a human or non-human animal.

In some cases, a method of the present disclosure comprises introducing into a eukaryotic cell in vitro: i) a recombinant expression vector comprising a nucleotide sequence encoding the EvolvR polypeptide (i.e., a EvolvR polypeptide comprising: i) a CRISPR-Cas effector polypeptide (e.g., a Slug-nCas9); and ii) two or more heterologous polypeptides, wherein one of the two or more heterologous polypeptides is an error-prone DNA polymerase and wherein one of the two or more heterologous polypeptides comprises an NES polypeptide); and ii) one or more guide RNAs. In some cases, the guide RNA is a single-molecule guide RNA (a “sgRNA”); i.e., where the guide RNA is a single RNA molecule. In some cases, the nucleotide sequence encoding the fusion protein is operably linked to a promoter. In some cases, the promoter is a constitutive promoter. In some cases, the promoter is a regulatable (e.g., inducible) promoter.

In some cases, a method of the present disclosure comprises introducing into a eukaryotic cell in vitro a recombinant expression vector that comprises: i) a first nucleotide sequence encoding one or more guide RNAs; and ii) a second nucleotide sequence encoding the EvolvR polypeptide (i.e., a EvolvR polypeptide comprising: i) a CRISPR-Cas effector polypeptide (e.g., a Slug-nCas9); and ii) two or more heterologous polypeptides, wherein one of the two or more heterologous polypeptides is an error-prone DNA polymerase and wherein one of the two or more heterologous polypeptides comprises an NES polypeptide). In some cases, the first nucleotide sequence is operably linked to a first promoter; and the second nucleotide sequence is operably linked to a second promoter.

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol Il, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), and the like.

Suitable expression vectors include viral expression vectors (e.g. viral vectors based on vaccinia virus; poliovirus; adenovirus; adeno-associated virus (AAV); SV40; herpes simplex virus; human immunodeficiency virus; a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector is a recombinant adeno-associated virus (AAV) vector. In some cases, a recombinant expression vector is a recombinant lentivirus vector. In some cases, a recombinant expression vector is a recombinant retroviral vector.

Suitable eukaryotic cells include in vitro cell lines, e.g., mammalian cell lines. Suitable mammalian cell lines include, but are not limited to, Hela cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK 293T cells, HLHepG2 cells, and the like.

Methods of introducing a nucleic acid (e.g., a recombinant expression vector) into a host cell are known in the art, and any convenient method can be used to introduce a nucleic acid into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. A EvolvR polypeptide and a guide RNA can be present in a composition with a lipid. A EvolvR polypeptide and a guide RNA can be present in a lipid nanoparticle. Other suitable compositions are known in the art.

In some cases, a method of the present disclosure for modifying a target viral nucleic acid in the cytoplasm of a eukaryotic cell comprises: A) introducing into the eukaryotic cell gene editing components, wherein the gene editing components comprise: a) a EvolvR polypeptide comprising: i) a CRISPR-Cas effector polypeptide (e.g., a Slug-nCas9); and ii) two or more heterologous polypeptides, wherein one of the two or more heterologous polypeptides is an error-prone DNA polymerase and wherein one of the two or more heterologous polypeptides comprises an NES polypeptide; and b) one or more guide nucleic acids, wherein one or more guide nucleic acids comprise: i) a targeting region that comprises a nucleotide sequence that binds to a target sequence in the target viral nucleic acid; and ii) a protein-binding region that binds to the CRISPR-Cas effector polypeptide, thereby generating a modified eukaryotic cell; and B) infecting the modified eukaryotic cell with a virus comprising the target viral nucleic acid, wherein the target viral nucleic acid is contacted with the gene editing components, and wherein said contacting provides for modification of the target viral nucleic acid. In some cases, the infection step (step B) is carried out after the introduction step (step A). In some cases, step B is carried out from 2 hours to 96 hours after step A. For example, in some cases, step B is carried out from 2 hours to 4 hours, from 4 hours to 8 hours, from 8 hours to 12 hours, from 12 hours to 18 hours, from 18 hours to 24 hours, from 24 hours to 36 hours, from 36 hours to 48 hours, from 48 hours to 72 hours, or from 72 hours to 96 hours, after step A.

### Kits

Provided are kits/systems for carrying out a subject method. Such kits comprise various combinations of components useful in any of the methods described elsewhere herein. In some embodiments a subject kit includes a subject EvolvR polypeptide and/or a nucleic acid encoding it.

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A composition comprising an EvolvR polypeptide that comprises:
a) a Staphylococcus lugdunensis nickase Cas9 (Slug-nCas9) capable of introducing a single-stranded break in a target DNA; and
b) an error-prone DNA polymerase capable of synthesizing a new strand on the target DNA.

2. The composition of 1, wherein the EvolvR polypeptide is a fusion polypeptide comprising the Slug-nCas9 fused to the DNA polymerase.

3. The composition of 1, wherein the Slug-nCas9 is fused to a first member of a dimerization pair and the DNA polymerase is fused to a second member of the dimerization pair.

4. The composition of 3, wherein the dimerization pair is a constitutive dimer pair.

5. The composition of 3, wherein the dimerization pair is an inducible dimer pair.

6. The composition of any one of 3 to 5, wherein the first and second members of the dimerization pair are selected from:
a) leucine zipper polypeptides;
b) FK506 binding protein (FKBP1A) and FKBP1A;
c) FKBP1A and calcineurin catalytic subunit A (CnA);
d) FKBP1A and cyclophilin;
e) FKBP1A and FKBP-rapamycin associated protein (FRB);
f) gyrase B (GyrB) and GyrB;
g) dihydrofolate reductase (DHFR) and DHFR;
h) DmrB and DmrB;
i) PYL and ABI;
j) Cry2 and CIB1;
k) GAI and GID1;
I) SpyCatcher and SpyTag; and
m) GFP1-10 and GFP11.

7. The composition of any one of 1 to 6, wherein the Slug-nCas9 recognizes an NNGR protospacer adjacent motif (PAM) site.

8. The composition of any one of 1 to 6, wherein the Slug-nCas9 recognizes an NNG PAM site.

9. The composition of any one of 1 to 8, wherein the single-stranded break is in the target DNA's complementary strand.

10. The composition of any one of 1 to 8, wherein the single-stranded break is in the target DNA's non-complementary strand.

11. The composition of any one of 1 to 10, wherein the DNA polymerase comprises an amino acid that is at least 85% identical to the Escherichia coli DNA polymerase I amino acid sequence of SEQ ID NO: 3.

12. The composition of any one of 1 to 10, wherein the DNA polymerase is a DNA polymerase beta, a DNA polymerase iota, a DNA polymerase nu, a DNA polymerase eta, or a DNA polymerase kappa.

13. The composition of any one of 1 to 12, wherein the DNA polymerase comprises D424A, I709N, and A759R mutations as numbered relative to SEQ ID NO: 3.

14. The composition of any one of 1 to 12, wherein the DNA polymerase comprises D424A, I709N, A759R, F742Y, and P796H mutations as numbered relative to SEQ ID NO: 3.

15. The composition of any one of 1 to 14, wherein the DNA polymerase does not include an N-terminal flap endonuclease domain.

16. The composition of any one of 1 to 15, wherein the DNA polymerase introduces a mutation in the new strand at a distance of from 1 nucleotide to 10,000 nucleotides from the single-stranded break in the target DNA.

17. The composition of 16, wherein the DNA polymerase introduces a mutation in the new strand at a distance of from 1 nucleotide to 150 nucleotides from the single-stranded break in the target DNA.

18. The composition of 17, wherein the DNA polymerase introduces a mutation in the new strand at a distance of from 1 nucleotide to 22 nucleotides from the single-stranded break in the target DNA.

19. The composition of any one of 1 to 18, wherein the EvolvR polypeptide exhibits a target mutation rate of from 10⁻² to 10⁻⁸, 10-8 to 10⁻⁷, 10-7 to 10⁻⁶, 10-6 to 10⁻⁵, 10-5 to 10⁻⁴, 10⁻⁴ to 10⁻³, or 10⁻³ to 10⁻² mutations per nucleotide per genome replication event.

20. The composition of any one of 1 to 19, wherein the EvolvR polypeptide comprises a nuclear localization signal.

21. The composition of any one of 1 to 19, wherein the EvolvR polypeptide comprises a nuclear export signal.

22. The composition of any one of 1 to 21, wherein the EvolvR polypeptide comprises a linker connecting the Slug-nCas9 and the DNA polymerase.

23. The composition of any one of 1 to 22, further comprising a guide RNA that comprises a nucleotide sequence that is complementary to a target sequence in the target DNA.

24. The composition of 23, wherein the guide RNA is a single-molecule guide RNA.

25. The composition of any one of 23 to 24, wherein the target sequence is 18nt to 22nt long.

26. The composition of 25, wherein the target sequence is 20nt long.

27. The composition of any one of 23 to 26, wherein the guide RNA comprises a 5′ terminal purine.

28. The composition of 27, wherein the 5′ terminal purine is a guanine.

29. The composition of any one of 23 to 28, wherein the guide RNA is complexed with the EvolvR polypeptide, thus forming a ribonucleoprotein complex (RNP).

30. A system comprising one or more nucleic acids encoding the EvolvR polypeptide of any one of 1 to 22.

31. The system of 30, wherein a nucleotide sequence encoding the Slug-nCas9, and a nucleotide sequence encoding the DNA polymerase are present on the same nucleic acid.

32. The system of 31, wherein the nucleotide sequence encoding the Slug-nCas9 and the nucleotide sequence encoding the DNA polymerase are: (i) in-frame with one another, thus encoding a fusion protein comprising the Slug-nCas9 fused to the DNA polymerase, and (ii) operably linked to a promoter functional in a eukaryotic cell.

33. The system of any one of 30 to 32, further comprising the guide RNA of any one of 23 to 28, or a nucleotide sequence encoding said guide RNA.

34. A cell comprising the composition of any one of 1 to 29 or the system of any one of 30 to 33.

35. A method of modifying a target DNA, the method comprising contacting the target DNA with the composition of any one of 23 to 29.

36. The method of 35, wherein the target DNA is present in a cell.

37. The method of 36, wherein said contacting comprises introducing the system of any one of 30 to 33 into the cell.

38. The method of 36 or 37, wherein the cell is a eukaryotic cell.

39. The method of 36 or 37, wherein the cell is an animal cell or a plant cell.

40. The method of 36 or 37, wherein the cell is a mammalian cell.

41. The method of any one of 35 to 40, wherein the target DNA is a target viral DNA in the cytoplasm of a eukaryotic cell.

42. The method of any one of 35 to 41, wherein the method is performed in vitro.

43. The method of any one of 35 to 41, wherein the method is performed in vivo.

### Experimental Examples

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: EvolvR Diversifies Mammalian Genomic Loci

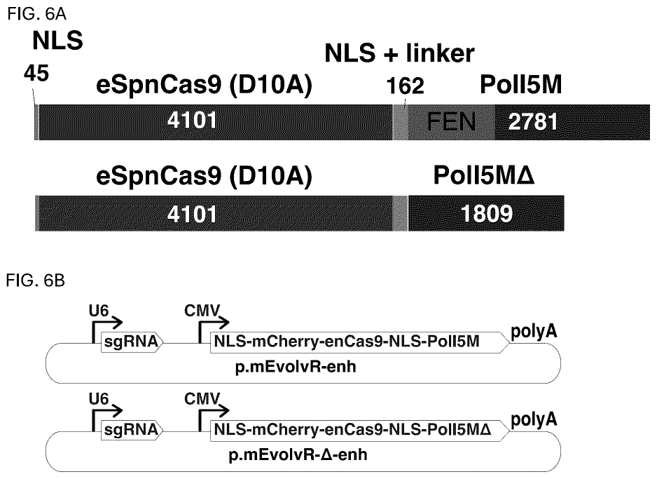

To test whether CRISPR-guided DNA polymerases (henceforth referred to as “EvolvR”) can diversify targeted genomic loci in human cells, the inventors designed an EvolvR construct. The EvolvR construct includes a nickase derived from an engineered SpCas9 variant (K848A, K1003A, R1060A) containing an additional RuvC-domain inactivating D10A missense mutation (D10A) fused to one of two variants of E. coli polymerase I harboring three (D424A, I709N, A759R) or five (D424A, I709N, A759R, F742Y, P796H) missense mutations that increase the polymerase's error rate named Poll3M and Poll5M, respectively¹³. To test EvolvR in mammalian cells, enCas9 was flanked with two nuclear localization sequences along with an mCherry fluorescent reporter tag (FIG. 6A). Additionally, a version of Poll5M without the N-terminal flap endonuclease domain (Poll5MA) was tested to determine whether the remaining Klenow fragment alone is sufficient for EvolvR-mediated mutagenesis.

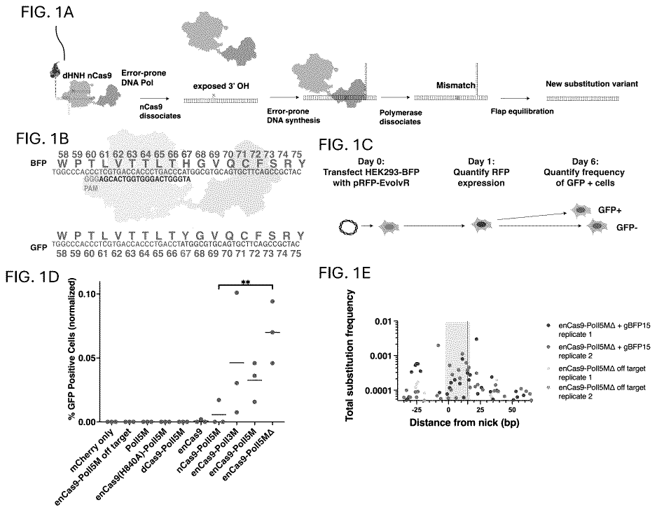

To rapidly and sensitively measure the frequency of single substitution variants generated by EvolvR, a fluorescent reporter for gene editing in HEK293 cells expressing a genomically-integrated, single-copy of a blue fluorescent protein (BFP) gene was used with a plasmid encoding strong expression of EvolvR (FIG. 6B) along with a 20 nt-long gRNA (henceforth referred to as “gBFP15”) directing a nick 15 bp upstream of a position that, upon undergoing an H67Y missense mutation resulting from replacing C199 with a T (henceforth referred to as 199C>T), results in a transition to green fluorescence (FIG. 1B). Six days after transfection, the frequency of GFP positive cells was measured by flow cytometry to compare the mutagenicity of different versions of EvolvR to controls (FIG. 1C). Conditions in which enCas9 and Poll5M were not fused or targeted to the BFP locus did not significantly increase the frequency of GFP positive cells when compared to cells expressing mCherry alone (FIG. 1D). Additionally, EvolvR constructed with wildtype nCas9 fused to Poll5M did not consistently generate GFP positive cells. In contrast, all three EvolvR variants consisting of enCas9 fused to Poll3M, Poll5M, or Poll5MΔ consistently generated GFP positive cells in all replicates. Notably, EvolvR lacking the flap endonuclease domain generated the highest mean frequency of GFP-expressing cells in the final population, though no differences in GFP positive cells generated between enCas9-Poll3M, enCas9-Poll5M, or enCas9-Poll5MΔ were statistically significant. These data suggest that Pol I's flap endonuclease domain is inessential for EvolvR's targeted mutagenicity in HEK293 cells, potentially due to redundancy with endogenous nuclear flap exonucleases. A flap endonuclease is set forth as SEQ ID NO: 22.

Having found that EvolvR elevates mutation rates of a specific nucleotide within a user-defined genomic locus, next generation sequencing was then used to quantify the diversity generated at the target locus (i.e. EvolvR's mutation window). HEK293 BFP cells were again transfected with plasmids encoding mCherry-tagged enCas9-Poll5MΔ and gBFP15 or a gRNA targeting safe harbor control locus AAVS1. To ensure normalization of substitution frequencies across samples and exclude untransfected cells from analysis, cells expressing EvolvR were isolated by FACS by gating for mCherry fluorescence. One week after this sort, the genomic DNA of biological duplicates were harvested from each condition and the frequency of substitutions at each position quantified using next generation sequencing of BFP amplicons. Coexpression of EvolvR and gBFP15 significantly elevated the substitution rate throughout the gRNA spacer and beyond compared to the off target negative control (FIG. 1E). EvolvR's elevated substitution rates extended from the nick to the end of the region quantified approximately 50 bp downstream of the nick, though the frequency of substitutions was markedly higher within the gRNA target sequence.

Intriguingly, though EvolvR generated mutations asymmetrically in the direction expected during error-prone DNA synthesis, mutations in the opposite direction upstream of the nick were also detectable. It is postulated that bidirectional mutagenesis occurs due to Pol I participation in DNA repair processes initiated during replication fork collisions with EvolvR-generated single-stranded breaks. Specifically, Pol I may initiate error-prone transcription from free 3′ ends remaining during MRE-mediated resection of double-stranded breaks, which are known to occur during replication fork collapse arising from single-stranded breaks^{27,28},

### Example 2: EvolvR-Based Screen Identifies Novel Drug-Resistant MAP2K1 Exon 6 Variants

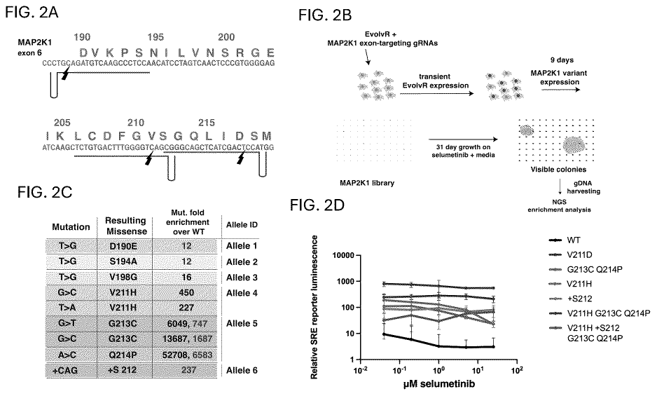

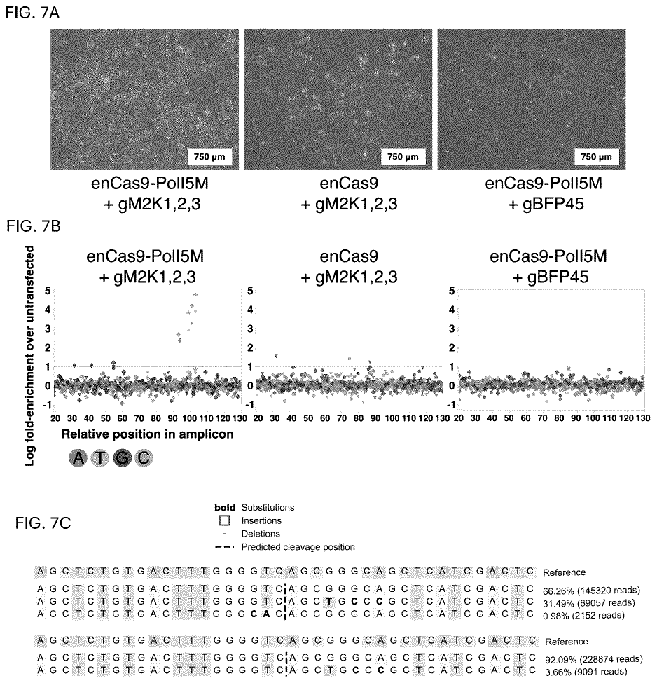

After demonstrating that EvolvR diversifies all four nucleotides within at least 50 bp of the nickase's cut site, EvolvR's capacity for generating novel gain of function variants within native genomic loci was tested by targeting EvolvR to exon 6 of MAP2K1 (FIG. 2A) and screening the resulting library for variants resistant to MAP2K1 inhibitor selumetinib, as A375 cells depend on this kinase's activity for replication³⁰. A375 cells were transiently transfected with a plasmid encoding enCas9-Poll5M and one of three gRNAs targeting three different loci along MAP2K1 exon 6, where mutations that confer resistance to selumetinib have been previously identified³¹. After transfection, cells were split into three separate cultures per condition and allowed to recover for six days before addition of 1 μM selumetinib (FIG. 2B). Non-target control cell cultures transfected with EvolvR and a gRNA targeting a nonexistent BFP gene (gBFP45) did not noticeably replicate and in one of three replicates were not sufficiently viable after passaging into selective media for harvesting DNA. In contrast, cultures transfected with EvolvR and three gRNAs targeting three nonoverlapping loci in exon 6 contained visible colonies at the end of the selection period (FIG. 7A).

Amplicon sequencing of MAP2K1 gDNA from cells transfected with EvolvR revealed seven variants, six of which were substitution variants, that were enriched by at least 10-fold over mean frequencies of those variants in a triplicate untreated, unselected control (FIG. 2C, FIGS. 7B and 7C). To validate the three most enriched drug-resistant MAP2K1 variants (V211H, G213C Q214P, +S212), each variant was cloned and HEK293T cells transiently transfected with the resulting expression plasmids. MAP2K1-ERK activity was measured in the presence of selumetinib using a reporter plasmid for SRE-controlled luciferase expression, previously used for measuring MAP2K1 activity in the presence of an inhibitor³² (FIG. 2D). As positive and negative controls, the newly discovered variants were tested against known selumetinib-resistant variant V211D³³ and a plasmid encoding wildtype MAP2K1, respectively. V211H, G213C/Q214P and +S212 all showed statistically significantly higher activity at all concentrations of selumetinib tested compared to wildtype. Additionally, V211H was generated in combination with G213C Q214P, and it was found that this variant's relative MAP2K1-ERK activity was approximately 2.3-fold higher than that of V211D and 205.3-fold higher than wildtype MAP2K1 under inhibition by 1 μM selumetinib. The absence of V211H, G213C, and Q214P triple missense variants in the selection is attributed to insufficient sampling of quintuple substitution variant space with a culture size of approximately 10⁷ cells. Remarkably, all six missense variants enriched have not previously been described and were all generated via substitutions not accessible with deaminases, revealing EvolvR's potential to access diversity spaces yet unexplored by current in vivo diversifiers. Furthermore, one of the seven variants generated by EvolvR was an insertion variant resulting from an in-frame insertion of an additional serine in between V211 and S212 (+S212), demonstrating the potential value of EvolvR's capacity to generate functional indel variants in addition to substitutions.

### Example 3: Nickase Mismatch Tolerance Dictates the Diversity of EvolvR-Mediated Substitutions within the gRNA Target Region

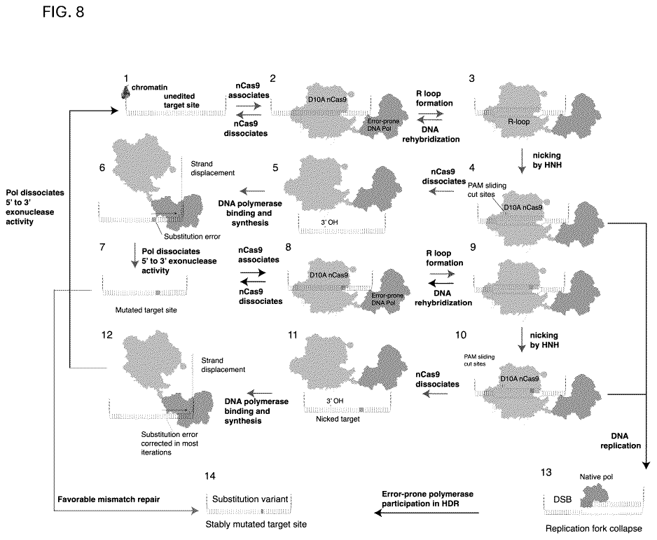

Though the known resistance-conferring T to A substitution in V211 was present and enriched by our screen, this substitution occurred only in combination with an additional G to C substitution in the codon encoding V211, resulting in a V211H missense mutation rather than the V211D mutation previously reported to confer resistance to selumetinib^{31,33}. To explain the absence of the single substitution variant V211D, it was first considered that SpCas9 frequently tolerates single mismatches within the 20 bp-long target region in a sequence-specific fashion, both within the seed region and PAM-distal nucleotides³⁴. Understanding that enCas9 frequently tolerates mismatches throughout the target strand led to the hypothesis that mutation-containing target strands are re-nicked and subsequently overwritten by Poll5M using the wildtype sequence as a template to “erase” any previously introduced mutations. In contrast, mutations that do not sufficiently disrupt R loop formation and subsequent nicking by enCas9 remain and are left to be processed by mismatch repair or “locked in” upon DNA replication. This mechanism is henceforth referred to as the “mismatch tolerance bias” (MTB) model for EvolvR's positional substitution biases (FIG. 8). The MTB model explains enCas9-Poll5M's apparently higher single substitution rates than wild type nCas9-Poll5M both in the fluctuation assays originally described in E. coli¹³ and in the BFP to GFP assay in HEK293 cells (FIG. 1D), especially as both assays measure the assay measures EvolvR's substitution rates in PAM-distal nucleotides, where eSpCas91.1's nicking fidelity is highest relative to wild type SpCas937.

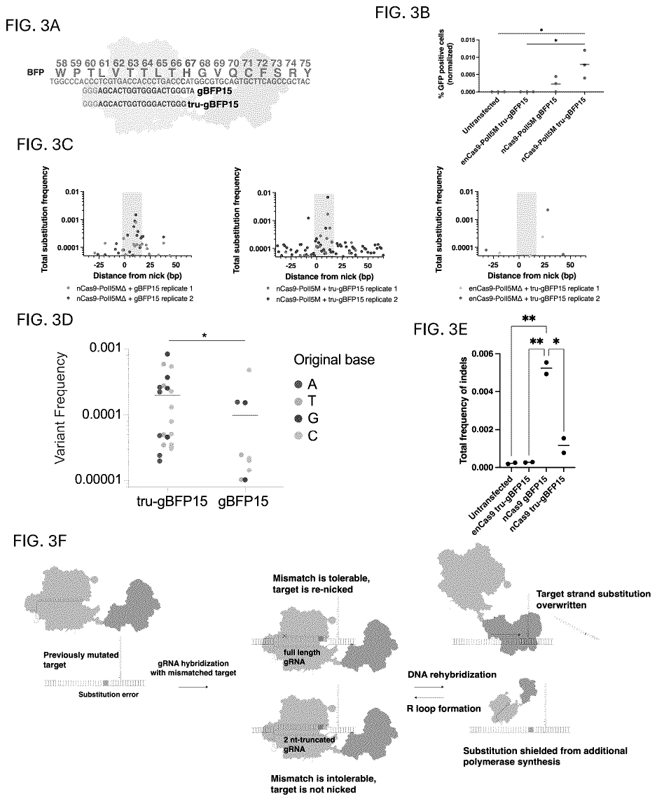

To test the validity of the MTB model, it was assessed whether improving the fidelity of nCas9 within EvolvR would improve its capacity to generate GFP positive cells. The use of truncated gRNAs decreases Cas9's off-target activity without decreasing its on-target efficiency for certain gRNAs, thereby improving Cas9's fidelity. Moreover, the efficiency of single-nucleotide genomic edits using Cas9-induced homology-directed repair (HDR) can be improved using 5′-truncated 18 nt gRNAs. To test whether the efficiency of EvolvR mutagenesis can also be improved by supplying nCas9 with truncated gRNAs, 18 nt-long gRNAs truncated by 2 nt at their 5′ end were used and enCas9-Poll5M activity was assessed with the BFP to GFP assay (FIG. 3A). The truncated guides elevated the frequency of GFP positive cells generated by nCas9-Poll5M's by 3.4-fold (FIG. 3B). In contrast, enCas9-Poll5M showed no evidence of mutagenesis when directed by a truncated gRNA, presumably due to eSpCas9 (1.1)'s sensitivity to the loss of PAM-distal gRNA-DNA matching³⁶.

As predicted by the MTB model, BFP to GFP conversion frequencies and amplicon sequencing of the target locus showed a 4.7-fold increase in the mean frequency of substitution variants within the gRNA target region (FIG. 3C) and a 2.7-fold increase in the number of unique variants within 18 bp from the PAM site (FIG. 3D) when nCas9-Poll5M was coexpressed with a truncated 18 nt-long gRNA relative to with a full-length 20 nt-long gRNA. In addition to increasing the total frequency of substitution variants, the use of a truncated gRNA also decreased the frequency of indel variants by 6-fold compared to cells treated with full-length gRNAs (FIG. 3E). This suggests that reducing the number of tolerable substitution variants facilitates the installation of substitution variants before the appearance of indels, which are known to be far more likely to be intolerable to Cas9 nuclease activity than substitutions^{37,40,41}. Additionally, as EvolvR can only be intolerant of substitutions that appear within the target sequence of its gRNA, its mutation rate should be markedly higher within the spacer region, consistent with the data shown here (FIG. 3C). Together, these data show that EvolvR's biases are heavily influenced by target sequence-specific and nickase-specific mismatch tolerance, and that current measurements of EvolvR's substitution biases are not reflective of polymerase error rates alone.

### Example 4: Incorporation of a PAM-Flexible Nickase with High Activity and Specificity Facilitates Consistent Mutagenesis of a Target Nucleotide with Most Overlapping gRNAs Targeting the Sense Strand

An attractive option for improving the utility of EvolvR is identifying or engineering RNA-guided nickases with reliably low mismatch tolerance and high activity when complexed with all or most gRNAs. Prior work has thus far only incorporated unengineered S. pyogenes nCas9 (D10A) and enhanced nicking Cas9 into EvolvR. However, other Cas9 variants with enhanced fidelities have been engineered, each of which could potentially endow EvolvR with more favorable on and off-target mutagenesis profiles. Therefore, a panel of engineered Cas9 variants was assessed using the BFP to GFP mutagenesis experiment.

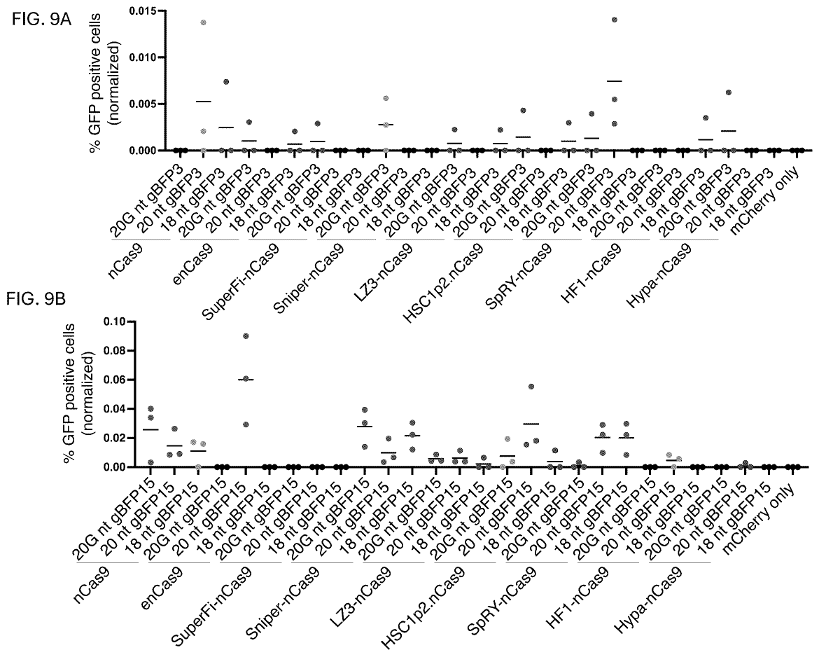

A panel of Cas9 nickases derived from various engineered Cas9 variants^{35,36,46,42,47,48,49} was examined for the capacity to generate GFP positive variants in all of three biological replicates (for consistency) using the BFP to GFP conversion assay with various gRNAs. To emulate variations in R loop formation dynamics, three gRNA variants were tested in each of two distinct spacer regions, where one gRNA generates a nick 3 bp away from 199C>T (gBFP3) and the other generates a nick 15 bp away (gBFP15). For each of the two spacer regions, three gRNA variants were tested: a 20 nt-long gRNA, an 18 nt-long gRNA, and a 20 nt-long gRNA with a guanine attached to its 5′ end, which is added to enhance gRNA expression from the human U6 promoter and initiate transcription from the first nucleotide of the gRNA sequence. The performance of all Cas9 variants tested was highly sensitive to both the spacer itself and the design of the gRNA used (FIGS. 9A and 9B). While nCas9, enCas9, Sniper-nCas9, LZ3-nCas9, HSC1.2-nCas9, and SpRY-nCas9 all generated GFP positive cells consistently in at least one of the three designs for gBFP15, only SpRY-nCas9 generated GFP positive cells for gBFP3. Furthermore, though SpRY-nCas9 generated GFP positive cells with 20 nt-long gBFP3, it only generated GFP positive cells in one replicate with 20 nt-long gBFP15. Therefore, EvolvR's capacity to diversify a particular nucleotide is largely dependent on nuclease properties and gRNA design.

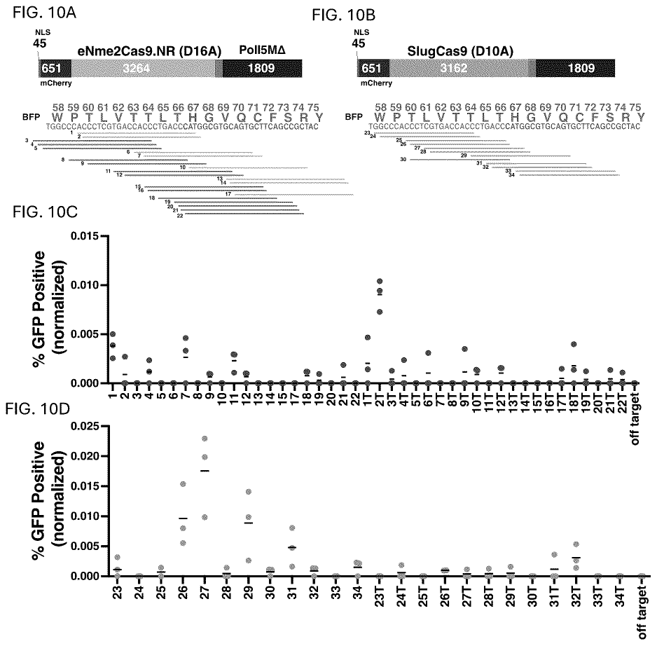

To compensate for gRNA variability in generating specific substitutions, additional nickases were considered that are highly active and specific and that in addition are PAM-flexible to maximize the number of gRNAs that overlap with a target locus. In particular, two RNA-guided nucleases known to exhibit improved specificity compared to SpCas951 were considered: Nme2Cas9⁵² and SlugCas9⁵³. To improve upon Nme2Cas9s somewhat restrictive NNNNCC PAM site, a variant of Nme2Cas9 known as eNme2-C.NR (henceforth referred to as eNme2.NR) engineered by phage-assisted continuous evolution (PACE) and shown to recognize NNNNCN PAM sites⁵⁴. SlugCas9 natively recognizes NNGR PAM sites⁵¹ was tested. BFP HEK293 cells were transfected with plasmids encoding EvolvR with either eNme2.NR-nCas9 or Slug-nCas9 as the nickase, in addition to one of a diverse panel of gRNAs (FIGS. 10A and 10B). 20 and 18-nt gRNAs were tested for Slug-nCas9, and 21 and 23-nt gRNAs were tested for eNme2.NR-nCas9, as Nme2Cas9 is conventionally paired with 23 nt gRNAs for optimal editing efficiency^{52,54}. Of the 44 gRNAs tested across 22 unique spacer sequences with eNme2.NR-nCas9, two generated GFP positive cells in all three biological replicates (FIG. 10A). The relatively low mutagenicity of eNme2.NR-nCas9-Poll5MΔ may be attributable to Nme2Cas9's relatively weak nuclease activity in mammalian cells⁵¹, though the degree to which eNme2.NR-Cas9's activity compares to wildtype Nme2Cas9 has not been rigorously quantified.

In contrast, of 24 gRNAs tested across 12 unique spacer sequences with Slug-nCas9, 6 generated GFP positive cells in all three replicates (FIG. 10B). Of these 6 gRNAs, 4 of them were 20 nt in length and represented 80% of the 20 nt gRNAs that both generated nicks upstream of 199C and overlapped with the 199C>T substitution. Through the MTB model, these data suggest that Slug-nCas9 is likely to exhibit low mismatch tolerance throughout its spacer region. This finding reveals that SlugCas9 may be the most specific and among the highest activity Cas9 nucleases reported to date.

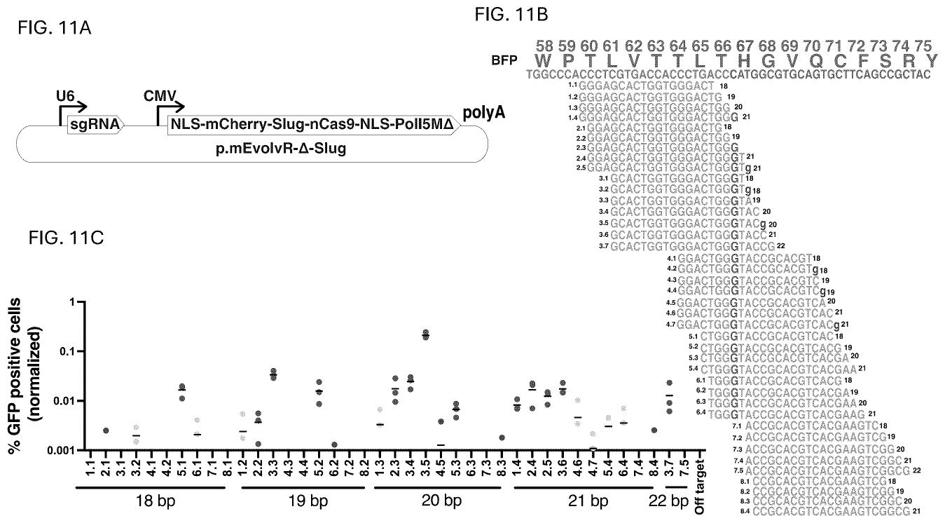

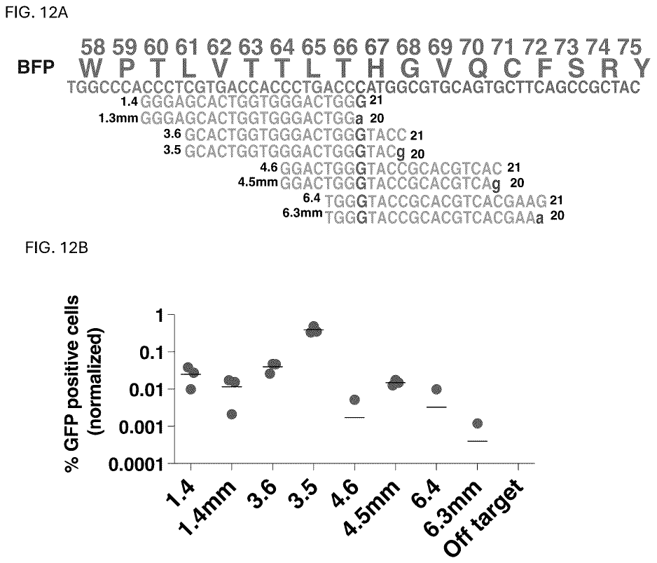

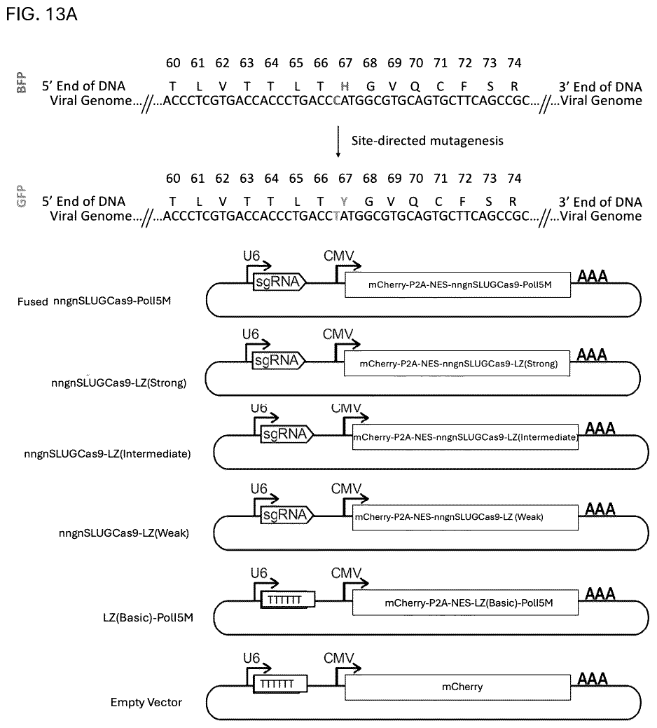

Given Slug-nCas9's markedly superior reliability in generating BFP to GFP substitutions when incorporated into EvolvR, Slug-nCas9-Poll5MA's performance was more thoroughly characterized using a panel of gRNAs targeting 8 different spacers near C199 and with various lengths. Because 5′ terminal purines offer tight control of gRNA transcriptional start sites and efficient transcription from the U6 promoter, the mutagenicity of each gRNA that does not natively possess a 5′ terminal purine (A or G) was tested with and without 5′ terminal guanines (FIGS. 11A and 11B). Of the 40 gRNAs tested, 12 generated GFP positive cells in all three biological replicates, all of which contained 199C>T within the spacer and downstream of the nick. These 12 gRNAs consist of a single gRNA with 18 bp of complementarity to the target site, three gRNAs with 19 bp of complementary, four with 20 bp of complementarity, and four with 21 bp of complementarity (FIG. 11C). To investigate whether adding mismatched 5′ terminal purines to gRNAs with 20 bp of complementary to the target site could predictably enhance or hinder EvolvR's mutation rates, EvolvR's mutation rates were compared when gRNAs 21 nt or 20 nt with an additional mismatched 5′ terminal purine were used to target BFP, it was found that the 5′ terminal mismatch could either increase or decrease EvolvR's efficiency in a gRNA-specific fashion (FIG. 12A). The data presented here suggest that the use of gRNAs with at least 20 bp of complementarity with the target strand, and optionally an added 5′ terminal guanine for optimal transcription, are suitable for mutagenesis with Slug-nCas9 EvolvR.

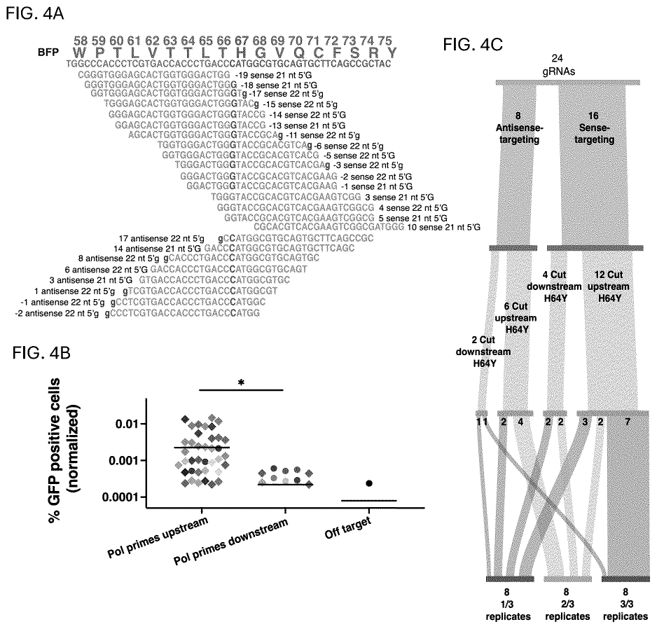

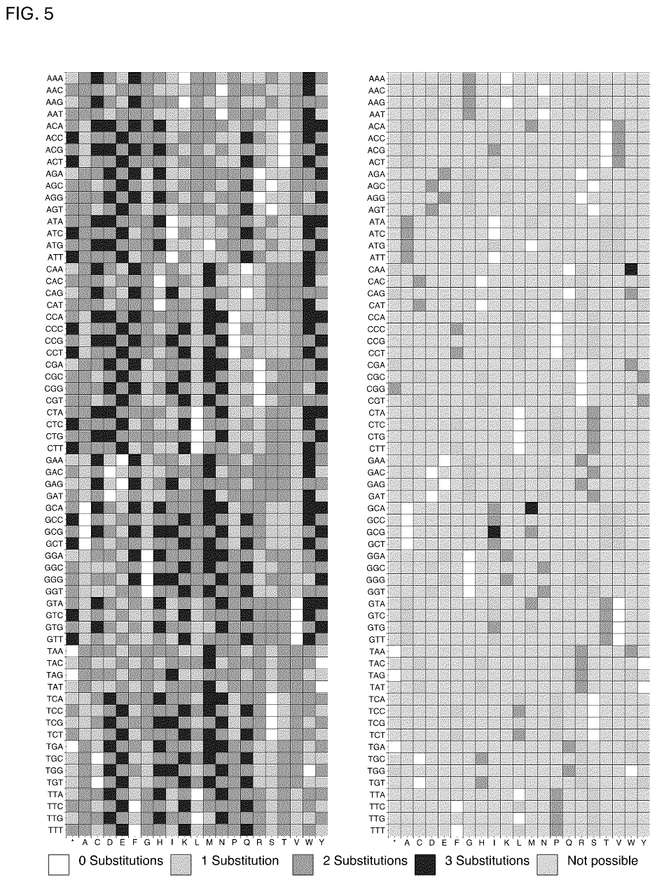

While Slug-nCas9 natively recognizes NNGR PAM sites, a variant of Slug-nCas9 recognizing NNG PAM sites was recently developed via PACE. Given that the human genome's GC content within 20 kb windows is at least 30%⁵⁵, NNG PAM site recognition would offer the capacity to diversify virtually any region of any gene of interest with EvolvR. Using 21-22 nt-long gRNAs (depending on whether a natural 5′ terminal guanine existed at the end of each gRNA, FIG. 4A), the reliability of NNG-Slug-nCas9-Poll5MΔ (SEQ ID NO: 25) in generating BFP to GFP edits was tested. Among the 24 gRNAs tested, ones that generated nicks upstream of C199 most efficiently generated GFP positive mutants, highlighting EvolvR's directionally asymmetrical mutation windows consistent with Pol I initiating DNA synthesis from the nick generated by Cas9 (FIG. 4B). Additionally, 7 out of 8 gRNAs that resulted in measurable frequencies of GFP positive cells in all three replicates targeted the sense strand, suggesting a strong dependence of EvolvR's mutagenesis on which strand is targeted (FIG. 4C). This data suggests that RNA Pol II collision with single-stranded breaks, initiating a process known as transcription-coupled repair whereby the nicked strand is removed and resynthesized by native polymerases⁵⁶, may limit the activity of EvolvR when targeted to the template strand. The seven gRNAs that consistently generated GFP positive cells in the sense strand represent 58% of the sense strand-targeting gRNAs that generate nicks upstream of the 199C>T substitution, indicating that at least one gRNA could efficiently generate a given substitution at a target locus given the high availability of NNG PAM sites in the sense strand. Finally, of the PAM sites differing from Slug-nCas9's native NNGR PAM site recognition within the pool of loci that were possible to test, all three were NNGT PAM sites. Of these three, two generated GFP positive cells consistently, confirming NNG-Slug-nCas9-Poll5MA's (SEQ ID NO: 25) capacity to recognize PAM sites other than NNGR. The capacity of NNG-Slug-nCas9 to generate productive nicks leading to substitutions as part of the EvolvR fusion protein represents a critical leap in EvolvR's utility as a genomic diversifier, effectively removing PAM availability as a limitation of the EvolvR technology's applicability.

In summary, the engineered EvolvR of the present disclosure diversifies all four nucleotides in mammalian genomic loci and generates sufficient diversity within native genomic loci to identify novel drug resistant double and triple substitution variants not reachable with base editors. Moreover, the data presented here elucidate for the first time that EvolvR's mutation rates are strongly dependent on gRNA design and nickase properties, likely arising from EvolvR's mismatch tolerance. With these properties in mind, significant engineering improvements were successfully introduced to EvolvR, including the identification of Slug-nCas9 as a specific and highly active nickase for improving EvolvR's reliability per gRNA and the adaptation of a PAM-flexible Slug-nCas9 variant that recognizes NNG PAM sites. The result is an efficient genetic diversifer that can be targeted to virtually any position in the human genome, enabling the exploration of previously unexamined functions in human cells and unlocking an unprecedented potential to engineer complex functionalities via directed evolution, such as fit-for-purpose immune cell differentiation, user-defined transmembrane receptor signaling, or metabolic pathways.

### Example 5: Materials and Methods

### Plasmid Construction

Plasmids used in transfections were assembled using Golden Gate cloning. Plasmids used for transient transfection of 293 BFP and A375 cells contained a ColE1 origin of replication and AmpR resistance cassette. Prior to transfection, plasmids contained an sfGFP expression cassette in between human U6 promoter and a gRNA scaffold flanked by BsmBI cut sites that would generate overhangs 5′GGTG and 5′TTTG or 5′GTTG depending on the gRNA scaffold sequence. Oligonucleotides containing gRNA sequences and the appropriate overhang sequences 5′CACC and 5′AAAC or 5′CAAC were annealed and knocked in by Golden Gate reaction.

### Mammalian Cell Culture

HEK293T and A375 cells were obtained from American Type Culture Collection (ATCC) and stored by the UC Berkeley Cell Culture Facility located in 336 Barker Hall, Berkeley, CA 94720. BFP HEK293 cells were generously donated by Jacob Corn's lab.

Cells were cultured in Dulbecco's modified Eagle's medium (DMEM) plus L-glutamine (Gibco) supplemented with 10% (v/v) fetal bovine serum (Gibco, qualified) and 1x antibiotic-antimycotic. Cells were incubated at a temperature of 37 degrees Celsius and at 5% CO₂ concentration.

### BFP to GFP Mutagenesis Experiment in BFP HEK293 Cells

Unless otherwise noted, cells were transfected using Mirus X2's transfection reagent according to the manufacturer's protocol. Briefly, transfection complexes were prepared by mixing DNA in a 3:1 μg DNA to μL Mirus X2 reagent ratio in 250 μL Opti-MEM serum-free medium. The mixture was incubated at room temperature for 15-30 minutes before dispensing on top of cells. Cells were harvested at 70-90% confluence and reverse transfected by seeding cells at 1 million cells/well in tissue-culture treated 12-well plates immediately before adding transfection mixtures.

16-24 hours after transfection, cells were trypsinized and harvested from 12-well plates and resuspended in 1 mL PBS. 250 μL were transferred to a 96-well plate for flow cytometry analysis on an Attune Nxt flow cytometer, while the remaining 750 μL were separated into triplicate 6-well plate wells by dispensing 250 μL into each well. 120-144 hours later, cells were once again harvested by trypsinization, resuspended in 300 μL PBS in 96-well plates, and analyzed on the Attune NxT. Transfection rates and GFP frequencies were determined by analysis on FlowJo of the YL2 and BL1 channels collected on the Attune NxT.

For high-throughput screens of various high-fidelity nickases shown in FIG. 9, transfections were scaled down to 48-well plates by seeding 30,000 cells in 48-well plates per condition one day prior to transfection. When the cells were 70-90% confluent, the cells were transfected with Mirus X2 reagent according to the manufacturer's protocol by mixing 390 ng DNA in a 1:3 ng DNA to μL transfection reagent ratio with 1.17 μL transfection reagent in 39 μL Opti-MEM reduced serum medium. The cells were transfected and incubated as described previously. 16-24 hours later, the cells were resuspended in 400 μL enzyme-free dissociation buffer (Gibco) and split into four 100 μL volumes, where three were passaged into 2 mL growth media in 6-well plates and the remaining 100 μL volume was analyzed for mCherry fluorescence on an Attune NxT flow cytometer. The cells were incubated at 5% CO₂ and 37 degrees Celsius for approximately 7 days to facilitate mutagenesis and were then harvested and analyzed by flow cytometry as previously described.

Next-Generation Sequencing Experiments with HEK293 BFP Cells

HEK293 BFP cells were seeded at approximately 5,000,000 cells per plate in 10 cm tissue culture plates. When cells reached approximately 70% confluence, transfection complexes were prepared by mixing 15 μg plasmid with 45 μL Mirus LT1 reagent ratio in 1.5 mL Opti-MEM serum-free medium. The mixture was incubated at room temperature for 15-30 minutes before dispensing on top of cells. Approximately 36 hours later, cells were trypsinized and resuspended in 350 μL PBS and passed through a cell strainer into a FACS tube. 100,000 mCherry positive cells from each transfection were sorted on a Sony SH800Z sorter into 2 mL growth media in 15 mL Falcon tubes for each biological replicate, seeded onto 10 cm plates, and incubated for two weeks.

The cells were resuspended in 200 μL PBS and gDNA was harvested using a Qiagen DNEasy kit according to the manufacturer's protocol.

### Library Preparation for Next Generation Sequencing of BFP

The BFP locus was amplified from 500 ng of gDNA for 20 cycles of PCR using oligonucleotides GCTCTTCCGATCTNNNNNACCCTGAAGTTCATCTGCACCA (SEQ ID NO: 185) and GCTCTTCCGATCTNNNNNTTGAAGAAGATGGTGCGCTCCT (SEQ ID NO: 186). The PCR product was purified using PCR Reaction Cleanup beads from the UC Berkeley Sequencing Facility located in 310 Barker Hall, Berkeley, CA 94720. The beads were warmed to room temperature and mixed thoroughly in 9:5 volumetric ratio with the PCR product and incubated at room temperature for 5 minutes. Beads were separated from the solution by placing them on magnetic racks and washed with 70% ethanol twice. Ethanol was removed and beads were allowed to dry at room temperature for 30 minutes. The beads were removed from the magnetic rack and thoroughly resuspended in water to elute PCR 1 product. The concentration of PCR 1 product was quantified with a Qubit fluorometer using the high-sensitivity DNA kit. 10 ng of PCR 1 product was added to 10 additional cycles of PCR, in which Illumina adapter sequences and unique dual index pairs were attached. The samples were delivered to the University of California Berkeley Vincent J. Coates Genomics Sequencing Laboratory, where the samples were purified via bead cleanup and size selected by Pippin Prep. The molar concentration of each library prep was quantified by qPCR prior to pooling. The library was sequenced using an Illumina MiSeq V2 150 paired-end kit on an Illumina MiSeq sequencer. Samples were demultiplexed using bcl2fastq (v2.20).

### Sequencing Data Analysis and Variant Calling

The last 5 nucleotides of each read were trimmed to eliminate low quality reads using seqtk. Trimmed fastq.gz files were merged using NGmerge and were filtered for any read pairs that were not perfectly matching with “-p 0”. The merged fastq files were aligned with the BFP reference sequence with bowtie2. The resulting BAM files were indexed and converted to SAM files using samtools. Mpileup files were generated from the resulting SAM files using samtools mpileup, filtering for bases calls with a quality score of at least 30 using-Q 30 and no mapping quality filter. Variant calling was performed by running mpileup files through SiNPle⁵⁷ using a theta value (prior probability of a substitution variant) of 0.9.

To reduce the influence of base calling errors and prior mutations on variant calling, the mean variant frequency at each position for two untransfected negative control replicates was subtracted from the frequency of that same variant in all other conditions for all possible variants. Then, substitutions appearing at less than 0.00001 frequency were set to 0 to filter out variants that do not appear in at least 1/100,000 reads.

### MAP2K1 Evolution

A375 cells were transfected using Mirus X2's transfection reagent according to the manufacturer's protocol. Briefly, cells were seeded in 6-well plates one day prior to transfection. Once they reached 70% confluence, transfection complexes were prepared by mixing 1 μg plasmid in 3 μL Mirus X2 reagent in 250 μL Opti-MEM serum-free medium. The mixture was incubated at room temperature for 15-30 minutes before dispensing on top of cells.

Approximately 60 hours later, the cells were trypsinized and split in a 1:4 fashion into separate 10 cm tissue culture plates, and the remaining cells were analyzed on a Sony SH800Z cell sorter to confirm the efficient transfection of each sample. After one week of growth, the cells were split at a 1:10 ratio into new 10 cm dishes with selective growth media containing 1 μM selumetinib. Selective media was replaced routinely every 48-72 hours until large colonies were visible by the naked eye (approximately 41 days later).

The cells remaining on each plate were harvested and gDNA was harvested with a Qiagen DNEasy kit according to the manufacturer's protocol.

### Library Preparation for MAP2K1 Evolution Experiment

A section of the MAP2K1 exon 6 locus was amplified from 360 ng of gDNA for 20 cycles of PCR using forward primer GCTCTTCCGATCTNNNNNCCCTCCTTTTCTATTTTCTCTTCCCTGCAG (SEQ ID NO: 187) and reverse primer GCTCTTCCGATCTNNNNNCCGACATGTAGGACCTTGTGCCC (SEQ ID NO: 188). The PCR product was purified using PCR Reaction Cleanup beads from the UC Berkeley Sequencing Facility located in 310 Barker Hall, Berkeley, CA 94720. The beads were warmed to room temperature and mixed thoroughly in 9:5 volumetric ratio with the PCR product and incubated at room temperature for 5 minutes. Beads were separated from the solution by placing them on magnetic racks and washed with 70% ethanol twice. Ethanol was removed and beads were allowed to dry at room temperature for 30 minutes. The beads were removed from the magnetic rack and thoroughly resuspended in water to elute PCR 1 product. The concentration of PCR 1 product was quantified with a Qubit fluorometer using the high-sensitivity DNA kit. 10 ng of PCR 1 product was applied to a second PCR, in which Illumina adapter sequences and unique dual index pairs were attached. The samples were delivered to the Innovative Genomic Institute Sequencing Core, where the samples the molar concentration of each library prep was quantified by qPCR prior to equimolar pooling. The library was sequenced using an Illumina MiSeq V2 150 paired-end kit on an Illumina MiSeq sequencer. Samples were demultiplexed using bcl2fastq (v2.20).

### Sequencing Analysis for MAP2K1 Evolution Experiment

Raw fastq.gz files were processed into SiNPLe variant calling files as described previously. To calculate fold-enrichment of each substitution, the frequency of each variant in EvolvR on target conditions were divided by the frequency of those variants in the untransfected controls not having undergone selection.

Allele tables were generated using CRISPRESSO version 1.0.13.

### SRE Reporter Assay

DNA encoding wildtype MAP2K1 from which the enriched MAP2K1 variants were cloned by PCR was ordered as a gBlock from Integrated DNA Technologies and cloned by Golden Gate into a simple mammalian expression cassette under the control of a CMV promoter (pMAP2K1). HEK293 cells were seeded at 30,000 cells per well in 96 well white assay plates. After overnight incubation, the cells were transfected with Lipofectamine 2000 according to the manufacturer's protocol with 50 ng pMAP2K1. Approximately 7 hours later, the cells were washed twice with PBS and incubated with media containing selumetinib for 12 hours. After 12 hours of incubation at 37 degrees Celsius and 5% CO₂, the cells were washed with twice PBS and their media was replaced with media containing 10 ng/ml human epidermal growth factor (hEGF) and 0.5% FBS. Approximately 6 hours later, MAP2K1 activity was measured using the SRE Reporter Kit (BPS Bioscience) according to the manufacturer's protocol using a Tecan Spark plate reader. To quantify MAP2K1 activity, background luminescence was determined by measuring the luminescence per the manufacturer's protocol and subtracting the background luminescence from all other reads. To determine relative MAP2K1 activity between wells, background-subtracted Firefly luciferase luminescence was divided by luminescence generated by Renilla luciferase, a luminescent reporter of transfection efficiency.

### REFERENCES

1. Cirino, P. C., Mayer, K. M. & Umeno, D. Generating Mutant Libraries Using Error-Prone PCR. in Directed Evolution Library Creation: Methods and Protocols (eds. Arnold, F. H. & Georgiou, G.) 3-9 (Humana Press, Totowa, NJ, 2003). doi: 10.1385/1-59259-395-X: 3.
2. Stemmer, W. P. C. Rapid evolution of a protein in vitro by DNA shuffling. Nature 370, 389-391 (1994).
3. Zheng, L., Baumann, U. & Reymond, J.-L. An efficient one-step site-directed and site-saturation mutagenesis protocol. Nucleic Acids Res. 32, e115-e115 (2004).
4. Chen, K. & Arnold, F. H. Engineering new catalytic activities in enzymes. Nat. Catal. 3, 203-213 (2020).
5. Buskirk, A. R., Ong, Y.-C., Gartner, Z. J. & Liu, D. R. Directed evolution of ligand dependence: Small-molecule-activated protein splicing. Proc. Natl. Acad. Sci. 101, 10505-10510 (2004).
6. Guntas, G., Mansell, T. J., Kim, J. R. & Ostermeier, M. Directed evolution of protein switches and their application to the creation of ligand-binding proteins. Proc. Natl. Acad. Sci. 102, 11224-11229 (2005).
7. Jäckel, C., Kast, P. & Hilvert, D. Protein Design by Directed Evolution. Annual Review of Biophysics vol. 37 153-173 (2008).
8. Hubbard, B. P. et al. Continuous directed evolution of DNA-binding proteins to improve TALEN specificity. Nat. Methods 12, 939-942 (2015).
9. Maheshri, N., Koerber, J. T., Kaspar, B. K. & Schaffer, D. V. Directed evolution of adeno-associated virus yields enhanced gene delivery vectors. Nat. Biotechnol. 24, 198-204 (2006).
10. Bartel, M. A., Weinstein, J. R. & Schaffer, D. V. Directed evolution of novel adeno-associated viruses for therapeutic gene delivery. Gene Ther. 19, 694-700 (2012).
11. Schieferecke, A. J. et al. Evolving membrane-associated accessory protein variants for improved adeno-associated virus production. Mol. Ther. 32, 340-351 (2024).
12. Ravikumar, A., Arzumanyan, G. A., Obadi, M. K. A., Javanpour, A. A. & Liu, C. C. Scalable, Continuous Evolution of Genes at Mutation Rates above Genomic Error Thresholds. Cell 175, 1946-1957.e13 (2018).
13. Halperin, S. O. et al. CRISPR-guided DNA polymerases enable diversification of all nucleotides in a tunable window. Nature 560, 248-252 (2018).
14. English, J. G. et al. VEGAS as a Platform for Facile Directed Evolution in Mammalian Cells. Cell 178, 748-761.e17 (2019).
15. Berman, C. M. et al. An Adaptable Platform for Directed Evolution in Human Cells. J. Am. Chem. Soc. 140, 18093-18103 (2018).
16. Finney-Manchester, S. P. & Maheshri, N. Harnessing mutagenic homologous recombination for targeted mutagenesis in vivo by TaGTEAM. Nucleic Acids Res. 41, e99-e99 (2013).
17. Hess, G. T. et al. Directed evolution using dCas9-targeted somatic hypermutation in mammalian cells. Nat. Methods 13, 1036-1042 (2016).
18. Ma, Y. et al. Targeted AID-mediated mutagenesis (TAM) enables efficient genomic diversification in mammalian cells. Nat. Methods 13, 1029-1035 (2016).
19. Zimmermann, A. et al. A Cas3-base editing tool for targetable in vivo mutagenesis. Nat. Commun. 14, 3389 (2023).
20. Molina, R. S. et al. In vivo hypermutation and continuous evolution. Nat. Rev. Methods Primer 2, 36 (2022).
21. Klenk, C. et al. A Vaccinia-based system for directed evolution of GPCRs in mammalian cells. Nat. Commun. 14, 1770 (2023).
22. Daniels, K. G. et al. Decoding CAR T cell phenotype using combinatorial signaling motif libraries and machine learning. Science 378, 1194-1200 (2022).
23. Sockolosky, J. T. et al. Selective targeting of engineered T cells using orthogonal IL-2 cytokine-receptor complexes. Science 359, 1037-1042 (2018).
24. Denes, C. E. et al. The VEGAS Platform Is Unsuitable for Mammalian Directed Evolution. ACS Synth. Biol. 11, 3544-3549 (2022).
25. Tou, C. J., Schaffer, D. V. & Dueber, J. E. Targeted Diversification in the S. cerevisiae Genome with CRISPR-Guided DNA Polymerase I. ACS Synth. Biol. 9, 1911-1916 (2020).
26. Gossing, M. et al. Multiplexed Guide RNA Expression Leads to Increased Mutation Frequency in Targeted Window Using a CRISPR-Guided Error-Prone DNA Polymerase in Saccharomyces cerevisiae. ACS Synth. Biol. 12, 2271-2277 (2023).
27. Pavani, R. et al. Structure and repair of replication-coupled DNA breaks. Science 0, eado3867.
28. Nicolette, M. L. et al. Mre11-Rad50-Xrs2 and Sae2 promote 5′ strand resection of DNA double-strand breaks. Nat. Struct. Mol. Biol. 17, 1478-1485 (2010).
29. Yoo, K. W., Yadav, M. K., Song, Q., Atala, A. & Lu, B. Targeting DNA polymerase to DNA double-strand breaks reduces DNA deletion size and increases templated insertions generated by CRISPR/Cas9. Nucleic Acids Res. 50, 3944-3957 (2022).
30. Gao, Y. et al. V211D Mutation in MEK1 Causes Resistance to MEK Inhibitors in Colon Cancer. Cancer Discov. 9, 1182-1191 (2019).
31. Emery, C. M. et al. MEK1 mutations confer resistance to MEK and B-RAF inhibition. Proc. Natl. Acad. Sci. 106, 20411-20416 (2009).
32. Chen, H. et al. Efficient, continuous mutagenesis in human cells using a pseudo-random DNA editor. Nat. Biotechnol. 38, 165-168 (2020).
33. Oddo, D. et al. Molecular Landscape of Acquired Resistance to Targeted Therapy Combinations in BRAF-Mutant Colorectal Cancer. Cancer Res. 76, 4504-4515 (2016).
34. Hua Fu, B. X., Hansen, L. L., Artiles, K. L., Nonet, M. L. & Fire, A. Z. Landscape of target: guide homology effects on Cas9-mediated cleavage. Nucleic Acids Res. 42, 13778-13787 (2014).
35. Slaymaker, I. M. et al. Rationally engineered Cas9 nucleases with improved specificity. Science 351, 84-88 (2016).
36. Chen, J. S. et al. Enhanced proofreading governs CRISPR-Cas9 targeting accuracy. Nature 550, 407-410 (2017).
37. Jones, S. K. et al. Massively parallel kinetic profiling of natural and engineered CRISPR nucleases. Nat. Biotechnol. 39, 84-93 (2021).
38. Fu, Y., Sander, J. D., Reyon, D., Cascio, V. M. & Joung, J. K. Improving CRISPR-Cas nuclease specificity using truncated guide RNAs. Nat. Biotechnol. 32, 279-284 (2014).
39. Lee, H. J., Kim, H. J. & Lee, S. J. Mismatch Intolerance of 5′-Truncated sgRNAs in CRISPR/Cas9 Enables Efficient Microbial Single-Base Genome Editing. Int. J. Mol. Sci. 22, (2021).
40. Rostain, W. et al. Cas9 off-target binding to the promoter of bacterial genes leads to silencing and toxicity. Nucleic Acids Res. 51, 3485-3496 (2023).
41. Cui, L. et al. A CRISPRi screen in E. coli reveals sequence-specific toxicity of dCas9. Nat. Commun. 9, 1912 (2018).
42. Pacesa, M. et al. Structural basis for Cas9 off-target activity. Cell 185, 4067-4081.e21 (2022).
43. Corsi, G. I. et al. CRISPR/Cas9 gRNA activity depends on free energy changes and on the target PAM context. Nat. Commun. 13, 3006 (2022).
44. Okafor, I. C. et al. Single molecule analysis of effects of non-canonical guide RNAs and specificity-enhancing mutations on Cas9-induced DNA unwinding. Nucleic Acids Res. 47, 11880-11888 (2019).
45. Gong, S., Yu, H. H., Johnson, K. A. & Taylor, D. W. DNA Unwinding Is the Primary Determinant of CRISPR-Cas9 Activity. Cell Rep. 22, 359-371 (2018).
46. Schmid-Burgk, J. L. et al. Highly Parallel Profiling of Cas9 Variant Specificity. Mol. Cell 78, 794-800.e8 (2020).
47. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
48. Kleinstiver, B. P. et al. High-fidelity CRISPR-Cas9 nucleases with no detectable genome-wide off-target effects. Nature 529, 490-495 (2016).
49. Zuo, Z. et al. Rational Engineering of CRISPR-Cas9 Nuclease to Attenuate Position-Dependent Off-Target Effects. CRISPR J. 5, 329-340 (2022).
50. Gao, Z., Harwig, A., Berkhout, B. & Herrera-Carrillo, E. Mutation of nucleotides around the +1 position of type 3 polymerase III promoters: The effect on transcriptional activity and start site usage. Transcription 8, 275-287 (2017).
51. Seo, S.-Y. et al. Massively parallel evaluation and computational prediction of the activities and specificities of 17 small Cas9s. Nat. Methods 20, 999-1009 (2023).
52. Edraki, A. et al. A Compact, High-Accuracy Cas9 with a Dinucleotide PAM for In Vivo Genome Editing. Mol. Cell 73, 714-726.e4 (2019).
53. Hu, Z. et al. Discovery and engineering of small SlugCas9 with broad targeting range and high specificity and activity. Nucleic Acids Res. 49, 4008-4019 (2021).
54. Huang, T. P. et al. High-throughput continuous evolution of compact Cas9 variants targeting single-nucleotide-pyrimidine PAMs. Nat. Biotechnol. 41, 96-107 (2023).
55. Lander, E. S. et al. Initial sequencing and analysis of the human genome. Nature 409, 860-921 (2001).
56. Hanawalt, P. C. & Spivak, G. Transcription-coupled DNA repair: two decades of progress and surprises. Nat. Rev. Mol. Cell Biol. 9, 958-970 (2008).
57. Ferretti, L., Tennakoon, C., Silesian, A., Freimanis, G. & Ribeca, P. SiNPle: Fast and Sensitive Variant Calling for Deep Sequencing Data. Genes 10, (2019).

### Example 6: Use of a Dimerization Pair to Express a Subject EvolvR Polypeptide

Nucleocytoplasmic large DNA viruses (NCLDVs) are a group of viruses harboring large (110 kpb-1.2 mbp) genomes comprised of double-stranded DNA that exist in the cytoplasm of eukaryotic cells. NCLDVs encompass Poxviridae, Asfaviridae, Iridoviridae, Ascovirida, Phycodnaviridae, Marseilleviridae, Pithoviridae, Mimiviridae, Pandoraviruses, Molliviruses, Faustoviruses, and other viral families. Some NCLDV viral families include species that can be used for biotechnological purposes, such as oncolytic virotherapies, vectored vaccines, immunotherapeutics, adjuvants, gene therapies, protein expression systems, or agricultural pest control agents. For example, vaccinia virus is a poxvirus that is useful as a vaccine and oncolytic virotherapy to treat cancer.

Biotechnologically useful properties of NCLDVs may be engineered using directed evolution, which is the iterative process of 1) generating diversity followed by 2) screening for improved function. Optimizable properties may include delivery efficiency, biodistribution, cell tropism and specificity, oncolytic activity, fusogenicity, immunogenicity, manufacturability, or other useful properties. While directed evolution has been widely used to optimize the properties of smaller viral vectors, NCLDVs possess multiple features that hinder targeted diversification in their genomes, which is the first step needed for directed evolution. For example, their large genome sizes, repetitive nucleotide sequence regions, reliance upon unique viral promoters and machinery to facilitate genomic replication in the cytoplasm, and limited homologous recombination efficiencies present barriers to implementing directed evolution to improve the biotechnological properties of NCLDVs.

To overcome the challenges of diversifying large viral vector genomes during replication in cells, error-prone DNA polymerases have fused to RNA-guided nickases to generate site-specific diversity in cytoplasmic DNA (see, e.g., WO2024011173). Clustered Regularly Interspaced Short Palindromic Repeats (CRISPR)-Cas systems comprise a CRISPR-associated (Cas) effector polypeptide and a guide nucleic acid. Such CRISPR-Cas systems can bind to and modify a targeted nucleic acid. The programmable nature of these CRISPR-Cas effector systems has facilitated their use as a versatile technology for use in, e.g., gene editing. CRISPR-Cas effector systems can be used to guide a fused polymerase to a site in the genome and generate a nick in the DNA that the error-prone polymerase can then extend off from to generate diversity. Using fluorescence-based and functional assays, it was shown this technology was able to generate site-specific diversity in poxvirus genomes, which replicate in the cytoplasm. Accordingly, RNA-guided nickase-polymerase fusion complexes with activity in the cytoplasm are herein referred to as “CytoEvolvR”. Note that in some cases, a nuclear export sequence (NES) may be appended to one or multiple parts of the CytoEvolvR machinery to promote localization in the cytoplasm.

Although RNA-guided nickase-polymerase fusion complexes have conferred editing of cytoplasmic DNA (see, e.g., WO2024011173), the large size of expression constructs needed to encode nickase-polymerase fusion proteins plus gRNA(s) presented challenges when trying to achieve high expression levels of EvolvR polypeptides, e.g., CytoEvolvR, or when incorporating EvolvR-encoding (e.g., CytoEvolvR-encoding) nucleic acid into a lentiviral vector to facilitate stable expression in primary or immortalized cells. To overcome this problem, the results here were generated using nucleic acids encoding the polymerase and RNA-guided nickase on separate plasmid constructs, and using a dimerization pair, which brings the two proteins together after translation. The results show that when the two separately produced protein products are subsequently bound together via a “dimerization pair,” higher activity (likely due to higher expression levels) is achieved. Protein domains that can be used as “dimerization pairs” may include, e.g., a pair of leucine zipper domains, SH3 domains, or other computationally or experimentally engineered protein domains that exhibit mutual affinity. An example of a SH3 100 nM Domain is SEQ ID NO: 30.

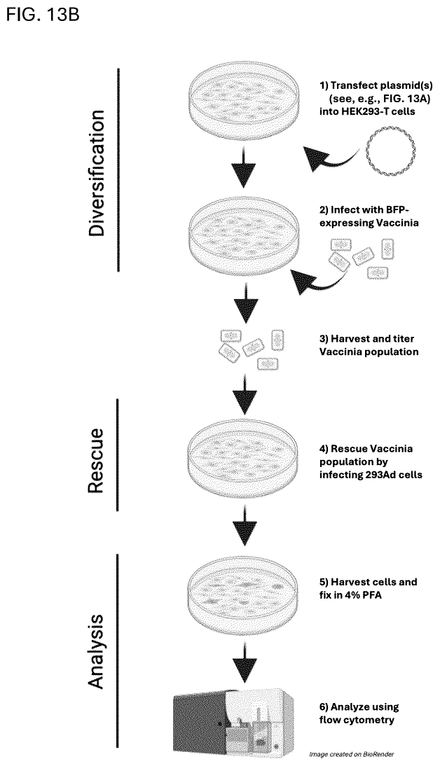

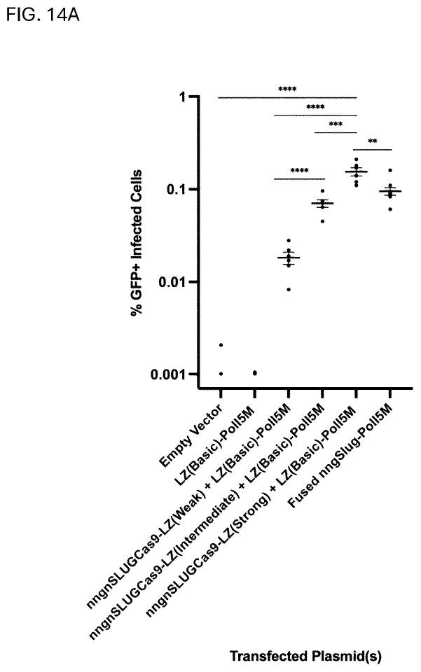

FIG. 13A-13B depict a system for characterizing diversification of user-defined loci in cytoplasmic DNA using the poxvirus vaccinia as a model.

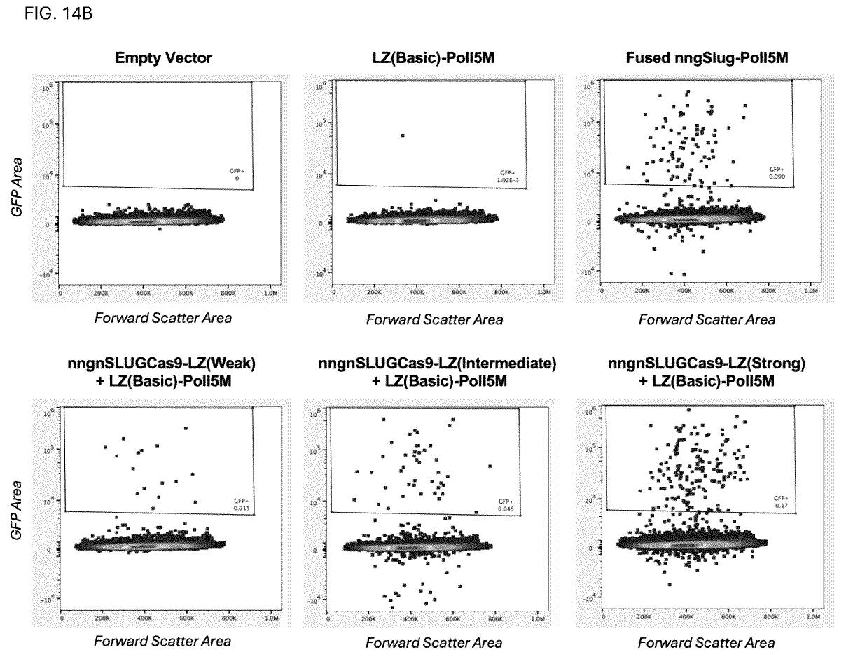

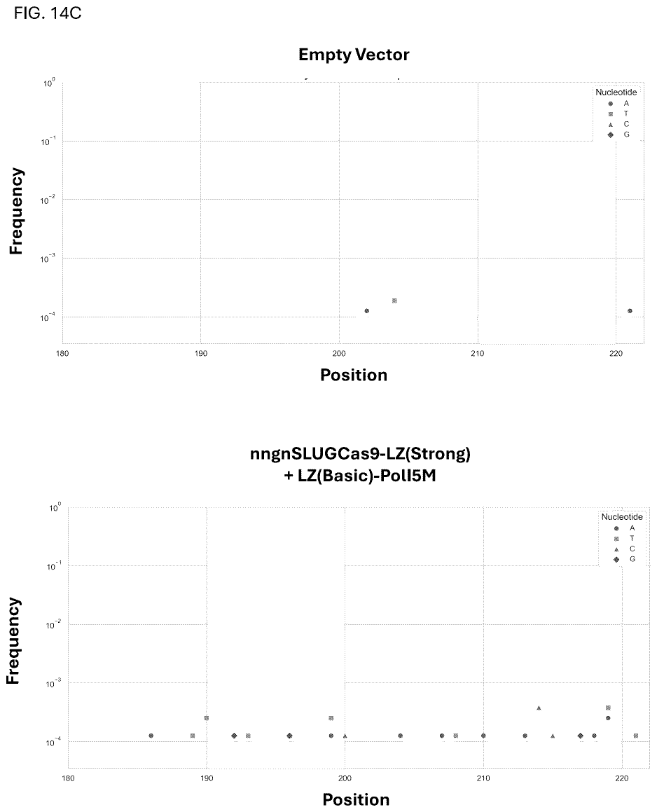

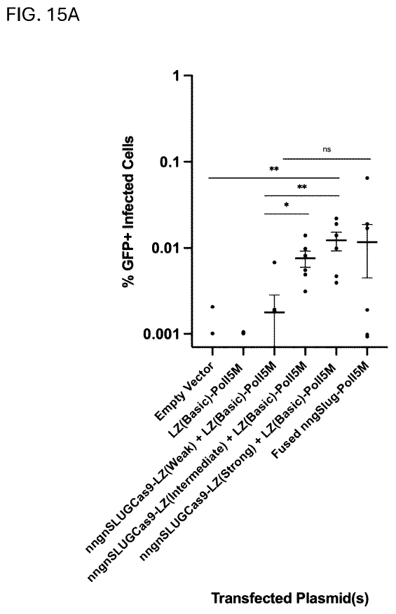

FIG. 14A-14C provide data showing that non-fused nickase+polymerase expressed from separate plasmids but subsequently bound using a leucine zipper and directed to the target site with an on-target gRNA confer diversification of a transgene encoded in vaccinia virus. FIG. 14A depicts the percent GFP-positive cells following passage of recombinant vaccinia virus containing a stably incorporated BFP gene in HEK293 cells that were previously transfected with a plasmid encoding 1) BFP-targeted gRNA with a target region corresponding to SEQ ID NO: 141 and NNG-PAM-utilizing nSlug-Cas9 (i.e., nicking Slug-cas9/the “nickase”), and a separate plasmid encoding 2) Poll5M (i.e., the “polymerase”). FIG. 14B depicts representative examples of the raw-data dot plots associated with the quantitative data depicted in FIG. 14A. FIG. 14C depicts a rare variant analysis following next-generation (Illumina amplicon) sequencing of the Empty Vector or nngnSLUGCas9-LZ (Strong)+LZ (Basic)-Poll5M samples from FIGS. 14A and 14B. An increased number of unique rare variants were observed in the nngnSLUGCas9-LZ (Strong)+LZ (Basic)-Poll5M sample relative to the Empty Vector control. These data (FIG. 14A-14C) provide an example that the stronger the affinity of the acidic peptide domain (which in this example is appended C-terminal to the nickase) for the basic peptide domain (which in this example is appended N-terminal to the polymerase), the higher the observed mutation rate. Thus, the mutation rate is tunable, i.e., controllable, by using different dimerization pairs.

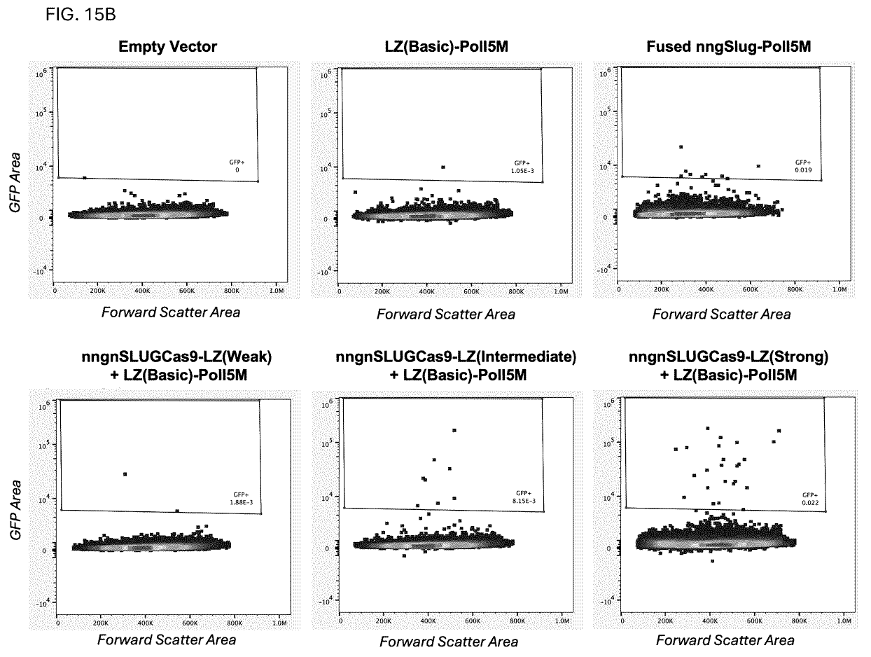

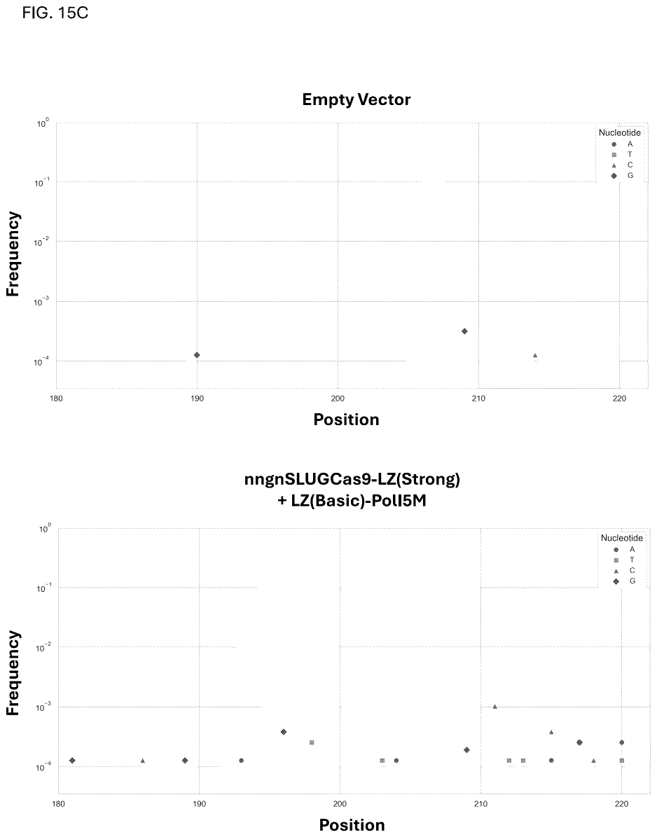

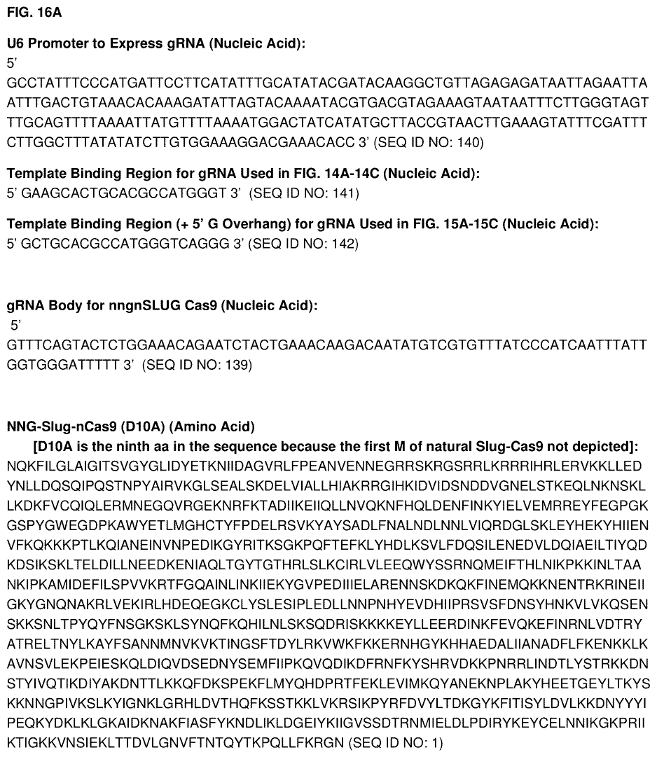

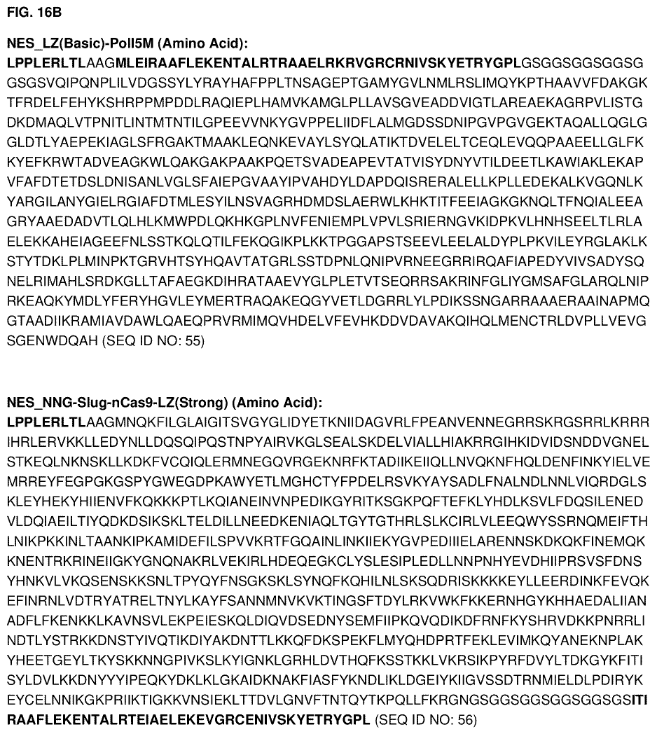

FIG. 15A-15C provide data showing that non-fused nickase+polymerase expressed from separate plasmids but subsequently bound using a leucine zipper and directed to the target site with a different on-target gRNA confer diversification of a transgene encoded in vaccinia virus. These data provide another example (using a different gRNA relative to that used in FIG. 14A-14C) that the stronger the affinity of the acidic peptide domain (which in this example is appended C-terminal to the nickase) for the basic peptide domain (which in this example is appended N-terminal to the polymerase), the higher the observed mutation rate. Thus, the mutation rate is tunable, i.e., controllable, by using different dimerization pairs.

FIG. 16A-16D provides example nucleic acid and protein sequences, many of which were used in the above examples.

Note: SEQ ID NO: 23 is T4 DNA ligase.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A composition comprising an EvolvR polypeptide that comprises:
a) a Staphylococcus lugdunensis nickase Cas9 (Slug-nCas9) capable of introducing a single-stranded break in a target DNA; and
b) an error-prone DNA polymerase capable of synthesizing a new strand on the target DNA.

2. The composition of claim 1, wherein the EvolvR polypeptide is a fusion polypeptide comprising the Slug-nCas9 fused to the DNA polymerase.

3. The composition of claim 1, wherein the Slug-nCas9 is fused to a first member of a dimerization pair and the DNA polymerase is fused to a second member of the dimerization pair, wherein the dimerization pair is a constitutive dimer pair or an inducible dimer pair.

4-5. (canceled)

6. The composition of claim 3, wherein the first and second members of the dimerization pair are selected from:
a) leucine zipper polypeptides;
b) FK506 binding protein (FKBP1A) and FKBP1A;
c) FKBP1A and calcineurin catalytic subunit A (CnA);
d) FKBP1A and cyclophilin;
e) FKBP1A and FKBP-rapamycin associated protein (FRB);
f) gyrase B (GyrB) and GyrB;
g) dihydrofolate reductase (DHFR) and DHFR;
h) DmrB and DmrB;
i) PYL and ABI;
j) Cry2 and CIB1;
k) GAI and GID1;
l) SpyCatcher and SpyTag; and
m) GFP1-10 and GFP11.

7. The composition of claim 1, wherein the Slug-nCas9 recognizes an NNGR protospacer adjacent motif (PAM) site, or an NNG PAM site.

8-10. (canceled)

11. The composition of claim 1, wherein the DNA polymerase comprises an amino acid that is at least 85% identical to the Escherichia coli DNA polymerase I amino acid sequence of SEQ ID NO: 3.

12. The composition of claim 1, wherein the DNA polymerase is a DNA polymerase beta, a DNA polymerase iota, a DNA polymerase nu, a DNA polymerase eta, or a DNA polymerase kappa.

13. The composition of claim 11, wherein the DNA polymerase comprises D424A, I709N, and A759R mutations as numbered relative to SEQ ID NO: 3, or comprises D424A, I709N, A759R, F742Y, and P796H mutations as numbered relative to SEQ ID NO: 3.

14. (canceled)

15. The composition of claim 1, wherein the DNA polymerase does not include an N-terminal flap endonuclease domain.

16. The composition of claim 1, wherein the DNA polymerase introduces a mutation in the new strand at a distance of from 1 nucleotide to 10,000 nucleotides from the single-stranded break in the target DNA, or introduces a mutation in the new strand at a distance of from 1 nucleotide to 150 nucleotides from the single-stranded break in the target DNA.

17-18. (canceled)

19. The composition of claim 1, wherein the EvolvR polypeptide exhibits a target mutation rate of from 10⁻⁸ to 10⁻² mutations per nucleotide per genome replication event.

20. The composition of claim 1, wherein the EvolvR polypeptide comprises a nuclear localization signal or a nuclear export signal.

21-22. (canceled)

23. The composition of claim 1, further comprising a guide RNA that comprises a nucleotide sequence that is complementary to a target sequence in the target DNA.

24-26. (canceled)

27. The composition of claim 23, wherein the guide RNA comprises a 5′ terminal purine.

28. (canceled)

29. The composition of claim 23, wherein the guide RNA is complexed with the EvolvR polypeptide, thus forming a ribonucleoprotein complex (RNP).

30. A system comprising one or more nucleic acids encoding the EvolvR polypeptide of claim 1.

31. The system of claim 30, wherein a nucleotide sequence encoding the Slug-nCas9, and a nucleotide sequence encoding the DNA polymerase are present on the same nucleic acid.

32. (canceled)

33. The system of claim 30, further comprising a guide RNA, or a nucleotide sequence encoding said guide RNA.

34. A cell comprising the composition of claim 1.

35. A method of modifying a target DNA, the method comprising contacting the target DNA with the composition of claim 1.

36. The method of claim 35, wherein the target DNA is present in a cell.

37. The method of claim 36, wherein said contacting comprises introducing into the cell: the composition of claim 1, or one or more nucleic acids, wherein said one or more nucleic acids encode the EvolvR polypeptide.

38. The method of claim 36, wherein the cell is a eukaryotic cell.

39-40. (canceled)

41. The method of claim 35, wherein the target DNA is a target viral DNA in the cytoplasm of a eukaryotic cell.

42-43. (canceled)


SEQUENCE LISTING (298 sequences)

SEQ ID NO: 1 - protein, 1053 aa, synthetic construct
NQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRRLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGRHLDVTHQFKSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGN

SEQ ID NO: 2 - protein, 1053 aa, synthetic construct
NQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 3 - protein, 927 aa, Escherichia coli
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 4 - protein, 927 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 5 - protein, 927 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 6 - protein, 927 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVFGLPLETVTSEQRRSAKRINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 7 - protein, 996 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKATETFGSWYQPKGGTEMFCHPRTGKPLPKYPRIKTPKVGGIFKKPKNKAQREGREPCELDTREYVAGAPYTPVEHVVFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVFGLPLETVTSEQRRSAKRINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 8 - protein, 927 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 9 - protein, 603 aa, synthetic construct
SYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 10 - protein, 574 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 11 - protein, 828 aa, synthetic construct
YSICVTRSCPVVCSKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFDSETSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 12 - protein, 682 aa, synthetic construct
NERLCYDIETDGLIEEVTKLHCIAIINVDTLEEALYADSLVMMTQGTIKDGLDRLNKASLTLGHNIIKYDHPVINKLTGYELPKDKSFDTLVASRLVFSNIKDIDQGNSGKYTLGKLFGSHSLEAWGIRLGGQQKMEYAPVIDPAQPVYDPTVKPKDAKKDPRWKGSIFTPMMGDYCMQDVRVNVELFRRLERKIHQLGYARSMLLEHEAAWVLAQQERNGFKFDEEKAIKLLGRLAGRREYLYNKLIETFGGWWVSDGVSVPARTINYKDPTKPSRVQGAPFTKVKWVDFNPSSRRHIIKVLTDRGWTPQEFTPSGEAKVDESILKELDFPEARLMAEWFLVQKRLGQLADGNQAWLNTVHPDGFIRGSVNPNGAVTGRATHSFPNVAQVPSCSAEYGAECRELFTVPDGWYLLGSDASGLELRCLANFMARYDDGKYIDVVLNGDIHWSNAQAAGFIPKGTIRDPHNPIHEEARRKAKTFIYAFLYGCGAELTGQQVGWTEEEYLNWKAKGAHKPIINRFKRQGKPWTREKICNILKGEEVQKNFMKGLPALKNLIDECKELHKEQGYIEGIDGRRIYTRSAHASLNTLLQGAGALVCKAWIVEIEKLAIAEGLKHGIDGDFMYCAWVHDEVQIACRTKEIAERIGQLCQVAMTNVEKEFNFVCRLDADFDIGKSWKETH

SEQ ID NO: 13 - protein, 1001 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVIGSIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGNELRCLAHFMARFDNGEYAHEILNGDIHTKNQIAAELPTRDNAKRFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 14 - protein, 739 aa, synthetic construct
EKLGVEPEEEGGGDDDEEDAEAWAMELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAKKGLIDYYLMPSLSTTSRSGKHSFKMKDTHMEDFPKDKETNRDFLPSGRIESTRTRESPLDTTNFSKEKDINEFPLCSLPEGVDQEVFKQLPVDIQEEILSGKSREKFQGKGSVSCPLHASRGVLSFFSKKQMQDIPINPRDHLSSSKQVSSVSPCEPGTSGFNSSSSSYMSSQKDYSYYLDNRLKDERISQGPKEPQGFHFTNSNPAVSAFHSFPNLQSEQLFSRNHTTDSHKQTVATDSHEGLTENREPDSVDEKITFPSDIDPQVFYELPEAVQKELLAEWKRAGSDFHIGHK

SEQ ID NO: 15 - protein, 712 aa, synthetic construct
ATGQDRVVALVDMDCFFVQVEQRQNPHLRNKPCAVVQYKSWKGGGIIAVSYEARAFGVTRSMWADDAKKLCPDLLLAQVRESRGKANLTKYREASVEVMEIMSRFAVIERASIDEAYVDLTSAVQERLQKLQGQPISADLLPSTYIEGLPQGPTTAEETVQKEGMRKQGLFQWLDSLQIDNLTSPDLQLTVGAVIVEEMRAAIERETGFQCSAGISHNKVLAKLACGLNKPNRQTLVSHGSVPQLFSQMPIRKIRSLGGKLGASVIEILGIEYMGELTQFTESQLQSHFGEKNGSWLYAMCRGIEHDPVKPRQLPKTIGCSKNFPGKTALATREQVQWWLLQLAQELEERLTKDRNDNDRVATQLVVSIRVQGDKRLSSLRRCCALTRYDAHKMSHDAFTVIKNCNTSGIQTEWSPPLTMLFLCATKFSASAPSSSTDITSFLSSDPSSLPKVPVTSSEAKTQGSGPAVTATKKATTSLESFFQKAAERQKVKEASLSSLTAPTQAPMSNSPSKPSLPFQTSQSTGTEPFFKQKSLLLKQKQLNNSSVSSPQQNPWSNCKALPNSLPTEYPGCVPVCEGVSKLEESSKATPAEMDLAHNSQSMHASSASKSVLEVTQKATPNPSLLAAEDQVPCEKCGSLVPVWDMPEHMDYHFALELQKSFLQPHSSNPQVVSAVSHQGKRNPKSPLACTNKRPRPEGMQTLESFFKPLTH

SEQ ID NO: 16 - protein, 869 aa, synthetic construct
DSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLEMSHKKSFFDKKRSERKWSHQDTFKCEAVNKQSFQTSQPFQVLKKKMNENLEISENSDDCQILTCPVCFRAQGCISLEALNKHVDECLDGPSISENFKMFSCSHVSATKVNKKENVPASSLCEKQDYEAHPKIKEISSVDCIALVDTIDNSSKAESIDALSNKHSKEECSSLPSKSFNIEHCHQNSSSTVSLENEDVGSFRQEYRQPYLCEVKTGQALVCPVCNVEQKTSDLTLFNVHVDVCLNKSFIQELRKDKFNPVNQPKESSRSTGSSSGVQKAVTRTKRPGLMTKYSTSKKIKPNNPKHTLDIFFK

SEQ ID NO: 17 - protein, 2589 aa, synthetic construct
NLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 18 - protein, 899 aa, synthetic construct
ENYEALVGFDLCNTPLSSVAQKIMSAMHSGDLVDSKTWGKSTETMEVINKSSVKYSVQLEDRKTQSPEKKDLKSLRSQTSRGSAKLSPQSFSVRLTDQLSADQKQKSISSLTLSSCLIPQYNQEASVLQKKGHKRKHFLMENINNENKGSINLKRKHITYNNLSEKTSKQMALEEDTDDAEGYLNSGNSGALKKHFCDIRHLDDWAKSQLIEMLKQAAALVITVMYTDGSTQLGADQTPVSSVRGIVVLVKRQAEGGHGCPDAPACGPVLEGFVSDDPCIYIQIEHSAIWDQEQEAHQQFARNVLFQTMKCKCPVICFNAKDFVRIVLQFFGNDGSWKHVADFIGLDPRIAAWLIDPSDATPSFEDLVEKYCEKSITVKVNSTYGNSSRNIVNQNVRENLKTLYRLTMDLCSKLKDYGLWQLFRTLELPLIPILAVMESHAIQVNKEEMEKTSALLGARLKELEQEAHFVAGERFLITSNNQLREILFGKLKLHLLSQRNSLPRTGLQKYPSTSEAVLNALRDLHPLPKIILEYRQVHKIKSTFVDGLLACMKKGSISSTWNQTGTVTGRLSAKHPNIQGISKHPIQITTPKNFKGKEDKILTISPRAMFVSSKGHTFLAADFSQIELRILTHLSGDPELLKLFQESERDDVFSTLTSQWKDVPVEQVTHADREQTKKVVYAVVYGAGKERLAACLGVPIQEAAQFLESFLQKYKKIKDFARAAIAQCHQTGCVVSIMGRRRPLPRIHAHDQQLRAQAERQAVNFVVQGSAADLCKLAMIHVFTAVAASHTLTARLVAQIHDELLFEVEDPQIPECAALVRRTMESLEQVQALELQLQVPLKVSLSAGRSWGHLVPLQEAWGPPPGPCRTESPSNSLAAPGSPASTQPPPLHFSPSFCL

SEQ ID NO: 19 - protein, 350 aa, Escherichia coli
RKIIHVDMDCFFAAVEMRDNPALRDIPIAIGGSRERRGVISTANYPARKFGVRSAMPTGMALKLCPHLTLLPGRFDAYKEASNHIREIFSRYTSRIEPLSLDEAYLDVTDSVHCHGSATLIAQEIRQTIFNELQLTASAGVAPVKFLAKIASDMNKPNGQFVITPAEVPAFLQTLPLAKIPGVGKVSAAKLEAMGLRTCGDVQKCDLVMLLKRFGKFGRILWERSQGIDERDVNSERLRKSVGVERTMAEDIHHWSECEAIIERLYPELERRLAKVKPDLLIARQGVKLKFDDFQQTTQEHVWPRLNKADLIATARKTWDERRGGRGVRLVGLHVTLLDPQMERQLVLGL

SEQ ID NO: 20 - protein, 64 aa, synthetic construct
MATVKFKYKGEEKEVDISKIKKVWRVGKMISFTYDEGGGKTGRGAVSEKDAPKELLQMLEKQKK

SEQ ID NO: 21 - protein, 764 aa, synthetic construct
SGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 22 - protein, 379 aa, synthetic construct
GIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 23 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 24 - protein, 2034 aa, synthetic construct
NQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRRLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGRHLDVTHQFKSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGNAYPYDVPDYASLGSGSPKKKRKVEDPKKKRKGSSETPGTSESATPEGGSGGSGSVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 25 - protein, 815 aa, synthetic construct
KSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGNAYPYDVPDYASLGSGSPKKKRKVEDPKKKRKGSSETPGTSESATPEGGSGGSGSSYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 26 - protein, 2034 aa, synthetic construct
NQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGNAYPYDVPDYASLGSGSPKKKRKVEDPKKKRKGSSETPGTSESATPEGGSGGSGSVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 27 - protein, 1710 aa, synthetic construct
NQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGNAYPYDVPDYASLGSGSPKKKRKVEDPKKKRKGSSETPGTSESATPEGGSGGSGSSYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 28 - protein, 444 aa, synthetic construct
EKLGVEPEEEGGGDDDEEDAEAWAMELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAK

SEQ ID NO: 29 - protein, 419 aa, synthetic construct
ELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAK

SEQ ID NO: 30 - protein, 11 aa, synthetic construct
PPPALPPKRRR

SEQ ID NO: 31 - protein, 1367 aa, Streptococcus pyogenes
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 32 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 33 - protein, 1299 aa, Francisella tularensis
SIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 34 - protein, 1306 aa, Acidaminococcus sp.
  source 1..1306: note = strain BV3L6
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 35 - protein, 1228 aa, Lachnospiraceae sp.
  source 1..1228: note = bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 36 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 37 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 38 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 39 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 40 - protein, 1082 aa, Prevotella pectinovora
NQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 41 - protein, 1320 aa, Prevotella ihumii
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSKFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKFKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSNSGTQFGGYLFRKKNEIGEYDYFLGISSKTQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIGNLLKTISCLKNKEEFHDLIKKDYQIFTEVQAVIDEICKQKTFIYFPVSNVELEKEMGDKDKPLCLFQISNKDLSFAKTFSANLRKKRGAENLHTMLFKALMSGNQDNLDLGSGAIFYRAKSLDGNKPTHPANEAIKCRNVANKDKVSLFTYDIYKNRRYMENKFLFHLSIVQNYKAAKDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGEMFVTRGQKIEKAVYQQFEKSLVDKLSYLVDKKRPYNELGGILKAYQLASSITKSNSDKQNGFLFYVPAWNTSKIDPVTGFTDLLRPKAMTIKEAQDFFGAFDNISYNDKGYFEFETNYDKFKIRMKGAQTRWTICTFGNRIKHKKDKNYWNYEEVELTEEFKKLFDKYDIDYRDGNLKEEILKIDNRKFFDALIKLLQLTLQLRNSDDKGNDYIVSPIANADGKFFNSNDGCKELPLDADANGAYNIARKGLWVVRQIKDKKDKISKLSNQEWLKFAQEKPYLK

SEQ ID NO: 42 - protein, 255 aa, Prevotella disiens
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDV

SEQ ID NO: 43 - protein, 1367 aa, synthetic construct
DKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 44 - protein, 1367 aa, synthetic construct
DKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 45 - protein, 1249 aa, synthetic construct
DKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 46 - protein, 1249 aa, synthetic construct
DKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 47 - protein, 75 aa, synthetic construct
TETFGSWYQPKGGTEMFCHPRTGKPLPKYPRIKTPKVGGIFKKPKNKAQREGREPCELDTREYVAGAPYTPVEHV

SEQ ID NO: 48 - protein, 510 aa, synthetic construct
ATGQDRVVALVDMDCFFVQVEQRQNPHLRNKPCAVVQYKSWKGGGIIAVSYEARAFGVTRSMWADDAKKLCPDLLLAQVRESRGKANLTKYREASVEVMEIMSRFAVIERASIDEAYVDLTSAVQERLQKLQGQPISADLLPSTYIEGLPQGPTTAEETVQKEGMRKQGLFQWLDSLQIDNLTSPDLQLTVGAVIVEEMRAAIERETGFQCSAGISHNKVLAKLACGLNKPNRQTLVSHGSVPQLFSQMPIRKIRSLGGKLGASVIEILGIEYMGELTQFTESQLQSHFGEKNGSWLYAMCRGIEHDPVKPRQLPKTIGCSKNFPGKTALATREQVQWWLLQLAQELEERLTKDRNDNDRVATQLVVSIRVQGDKRLSSLRRCCALTRYDAHKMSHDAFTVIKNCNTSGIQTEWSPPLTMLFLCATKFSASAPSSSTDITSFLSSDPSSLPKVPVTSSEAKTQGSGPAVTATKKATTSLESFFQKAAERQKVKEASLSSLTAPTQAPMSN

SEQ ID NO: 49 - protein, 559 aa, synthetic construct
DSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLE

SEQ ID NO: 50 - skipped

SEQ ID NO: 51 - protein, 43 aa, synthetic construct
ITIRAAFLEKENTALRTEIAELEKEVGRCENIVSKYETRYGPL

SEQ ID NO: 52 - protein, 43 aa, synthetic construct
ITIRAAFLEKENTALRTENAELEKEVGRCENIVSKYETRYGPL

SEQ ID NO: 53 - protein, 11 aa, synthetic construct
PPAALAPKRRR

SEQ ID NO: 54 - protein, 44 aa, synthetic construct
MLEIRAAFLEKENTALRTRAAELRKRVGRCRNIVSKYETRYGPL

SEQ ID NO: 55 - protein, 999 aa, synthetic construct
LPPLERLTLAAGMLEIRAAFLEKENTALRTRAAELRKRVGRCRNIVSKYETRYGPLGSGGSGGSGGSGGSGSVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 56 - protein, 1125 aa, synthetic construct
LPPLERLTLAAGMNQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRRLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGRHLDVTHQFKSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGNGSGGSGGSGGSGGSGSITIRAAFLEKENTALRTEIAELEKEVGRCENIVSKYETRYGPL

SEQ ID NO: 57 - protein, 1125 aa, synthetic construct
LPPLERLTLAAGMNQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRRLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGRHLDVTHQFKSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGNGSGGSGGSGGSGGSGSITIRAAFLEKENTALRTENAELEKEVGRCENIVSKYETRYGPL

SEQ ID NO: 58 - protein, 1100 aa, synthetic construct
LPPLERLTLAAGMNQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRRLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGRHLDVTHQFKSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGNGSGGSGGSGGSGGSGSGSGSGSGPPAALAPKRRR

SEQ ID NO: 59 - protein, 2009 aa, synthetic construct
LPPLERLTLAAGMNQKFILGLAIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRRLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGRHLDVTHQFKSSTKKLVKRSIKPYRFDVYLTDKGYKFITISYLDVLKKDNYYYIPEQKYDKLKLGKAIDKNAKFIASFYKNDLIKLDGEIYKIIGVSSDTRNMIELDLPDIRYKEYCELNNIKGKPRIIKTIGKKVNSIEKLTTDVLGNVFTNTQYTKPQLLFKRGNGSGGSGGSGGSGGSGSVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYARGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQNELRIMAHLSRDKGLLTAFAEGKDIHRATAAEVYGLPLETVTSEQRRSAKRINFGLIYGMSAFGLARQLNIPRKEAQKYMDLYFERYHGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 60 - skipped

SEQ ID NO: 61 - protein, 9 aa, synthetic construct
LPPLERLTL

SEQ ID NO: 62 - protein, 10 aa, synthetic construct
LALKLAGLDL

SEQ ID NO: 63 - protein, 14 aa, synthetic construct
MEELSQALASSFSV

SEQ ID NO: 64 - protein, 14 aa, synthetic construct
EAETVSAMALLSVG

SEQ ID NO: 65 - protein, 14 aa, synthetic construct
ELDELMASLSDFKF

SEQ ID NO: 66 - protein, 11 aa, synthetic construct
VDQLRLERLQI

SEQ ID NO: 67 - protein, 9 aa, synthetic construct
IDLSGLTLQ

SEQ ID NO: 68 - protein, 10 aa, synthetic construct
LRALERLQID

SEQ ID NO: 69 - protein, 10 aa, synthetic construct
LQKKLEELEL

SEQ ID NO: 70 - protein, 10 aa, synthetic construct
MQELSNILNL

SEQ ID NO: 71 - protein, 10 aa, synthetic construct
LCQAFSDVIL

SEQ ID NO: 72 - protein, 17 aa, synthetic construct
RTFDMHSLESSLIDIMR

SEQ ID NO: 73 - protein, 17 aa, synthetic construct
TNLEALQKKLEELELDE

SEQ ID NO: 74 - protein, 17 aa, synthetic construct
RSFEMTEFNQALEEIKG

SEQ ID NO: 75 - protein, 12 aa, synthetic construct
PLQLPPLERLTL

SEQ ID NO: 76 - protein, 12 aa, synthetic construct
NELALKLAGLDI

SEQ ID NO: 77 - protein, 15 aa, synthetic construct
ERFEMFRELNEALEL

SEQ ID NO: 78 - protein, 15 aa, synthetic construct
DHAEKVAEKLEALSV

SEQ ID NO: 79 - protein, 15 aa, synthetic construct
QLVEELLKIICAFQL

SEQ ID NO: 80 - protein, 15 aa, synthetic construct
TNLEALQKKLEELEL

SEQ ID NO: 81 - protein, 15 aa, synthetic construct
DVKEEMTSALATMRV

SEQ ID NO: 82 - protein, 15 aa, synthetic construct
STNGSLAAEFRHLQL

SEQ ID NO: 83 - protein, 15 aa, synthetic construct
PSVQELTEQIHRLLM

SEQ ID NO: 84 - protein, 15 aa, synthetic construct
MNFKELKDFLKELNI

SEQ ID NO: 85 - protein, 15 aa, synthetic construct
ENFEILMKLKESLEL

SEQ ID NO: 86 - protein, 15 aa, synthetic construct
FETVYELTKMCTIRM

SEQ ID NO: 87 - protein, 15 aa, synthetic construct
SGKASSSLGLQDFDL

SEQ ID NO: 88 - protein, 15 aa, synthetic construct
PKYSDIDVDGLCSEL

SEQ ID NO: 89 - protein, 18 aa, synthetic construct
ANDVDLACTPTDVRDVDI

SEQ ID NO: 90 - skipped

SEQ ID NO: 91 - protein, 7 aa, synthetic construct
PKKKRKV

SEQ ID NO: 92 - protein, 16 aa, synthetic construct
KRPAATKKAGQAKKKK

SEQ ID NO: 93 - protein, 9 aa, synthetic construct
PAAKRVKLD

SEQ ID NO: 94 - protein, 11 aa, synthetic construct
RQRRNELKRSP

SEQ ID NO: 95 - protein, 38 aa, synthetic construct
NQSSNFGPMKGGNFGGRSSGPYGGGGQYFAKPRNQGGY

SEQ ID NO: 96 - protein, 42 aa, synthetic construct
RMRIZFKNKGKDTAELRRRRVEVSVELRKAKKDEQILKRRNV

SEQ ID NO: 97 - protein, 8 aa, synthetic construct
VSRKRPRP

SEQ ID NO: 98 - protein, 8 aa, synthetic construct
PPKKARED

SEQ ID NO: 99 - protein, 8 aa, synthetic construct
PQPKKKPL

SEQ ID NO: 100 - protein, 12 aa, synthetic construct
SALIKKKKKMAP

SEQ ID NO: 101 - protein, 5 aa, synthetic construct
DRLRR

SEQ ID NO: 102 - protein, 7 aa, synthetic construct
PKQKKRK

SEQ ID NO: 103 - protein, 10 aa, synthetic construct
RKLKKKIKKL

SEQ ID NO: 104 - protein, 10 aa, synthetic construct
REKKKFLKRR

SEQ ID NO: 105 - protein, 20 aa, synthetic construct
KRKGDEVDGVDEVAKKKSKK

SEQ ID NO: 106 - protein, 17 aa, synthetic construct
RKCLQAGMNLEARKTKK

SEQ ID NO: 107 - protein, 30 aa, synthetic construct
MDSLLMNRRKFLYQFKNVRWAKGRRETYLC

SEQ ID NO: 108 - protein, 5 aa, synthetic construct
GSGGS

SEQ ID NO: 109 - protein, 6 aa, synthetic construct
GGSGGS

SEQ ID NO: 110 - protein, 5 aa, synthetic construct
GGGGS

SEQ ID NO: 111 - protein, 4 aa, synthetic construct
GGSG

SEQ ID NO: 112 - protein, 5 aa, synthetic construct
GGSGG

SEQ ID NO: 113 - protein, 5 aa, synthetic construct
GSGSG

SEQ ID NO: 114 - protein, 5 aa, synthetic construct
GSGGG

SEQ ID NO: 115 - protein, 5 aa, synthetic construct
GGGSG

SEQ ID NO: 116 - protein, 5 aa, synthetic construct
GSSSG

SEQ ID NO: 117 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 118 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 119 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 120 - RNA, 22 nt, synthetic construct
  source 1..22: mol_type = other RNA
gttttagagctatgctgttttg

SEQ ID NO: 121 - RNA, 12 nt, synthetic construct
  source 1..12: mol_type = other RNA
gttttagagcta

SEQ ID NO: 122 - RNA, 70 nt, synthetic construct
  source 1..70: mol_type = other RNA
aaacagcatagcaagttaaaataaggctagtccgttatcaacttgaaaaagtggcaccgagtcggtgctt

SEQ ID NO: 123 - RNA, 42 nt, synthetic construct
  source 1..42: mol_type = other RNA
gttttagagctagaaatagcaagttaaaataaggctagtccg

SEQ ID NO: 124 - RNA, 78 nt, synthetic construct
  source 1..78: mol_type = other RNA
gttttagagctagaaatagcaagttaaaataaggctagtccgttatcaacttgaaaaagtggcaccgagtcggtgctt

SEQ ID NO: 125 - RNA, 20 nt, synthetic construct
  source 1..20: mol_type = other RNA
aatttctactaagtgtagat

SEQ ID NO: 126 - RNA, 19 nt, synthetic construct
  source 1..19: mol_type = other RNA
aatttctactcttgtagat

SEQ ID NO: 127 - RNA, 19 nt, synthetic construct
  source 1..19: mol_type = other RNA
aatttctactgttgtagat

SEQ ID NO: 128 - RNA, 19 nt, synthetic construct
  source 1..19: mol_type = other RNA
aatttctactattgtagat

SEQ ID NO: 129 - RNA, 20 nt, synthetic construct
  source 1..20: mol_type = other RNA
aatttctactgtttgtagat

SEQ ID NO: 130 - RNA, 19 nt, synthetic construct
  source 1..19: mol_type = other RNA
aatttctactgttgtagat

SEQ ID NO: 131 - RNA, 19 nt, synthetic construct
  source 1..19: mol_type = other RNA
aatttctactattgtagat

SEQ ID NO: 132 - RNA, 36 nt, synthetic construct
  source 1..36: mol_type = other RNA
gtaagagactacctctatatgaaagaggactaaaac

SEQ ID NO: 133 - RNA, 36 nt, synthetic construct
  source 1..36: mol_type = other RNA
gatatagaccaccccaatatcgaaggggactaaaac

SEQ ID NO: 134 - RNA, 35 nt, synthetic construct
  source 1..35: mol_type = other RNA
atttagaccaccccaaaaatgaaggggactaaaac

SEQ ID NO: 135 - RNA, 30 nt, synthetic construct
  source 1..30: mol_type = other RNA
gaccaccccaaaaatgaaggggactaaaac

SEQ ID NO: 136 - RNA, 36 nt, synthetic construct
  source 1..36: mol_type = other RNA
gatttagactaccccaaaaacgaaggggactaaaac

SEQ ID NO: 137 - RNA, 32 nt, synthetic construct
  source 1..32: mol_type = other RNA
gtcacaactcccatgtaggcggagactgcaac

SEQ ID NO: 138 - RNA, 37 nt, synthetic construct
  source 1..37: mol_type = other RNA
ggatttagagtaccccaaaaatgaaggggactaaaac

SEQ ID NO: 139 - RNA, 81 nt, synthetic construct
  source 1..81: mol_type = other RNA
gtttcagtactctggaaacagaatctactgaaacaagacaatatgtcgtgtttatcccatcaatttattggtgggattttt

SEQ ID NO: 140 - DNA, 245 bp, synthetic construct
  source 1..245: mol_type = other DNA
gcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 141 - DNA, 21 bp, synthetic construct
  source 1..21: mol_type = other DNA
gaagcactgcacgccatgggt

SEQ ID NO: 142 - DNA, 21 bp, synthetic construct
  source 1..21: mol_type = other DNA
gctgcacgccatgggtcaggg

SEQ ID NO: 143 - protein, 43 aa, synthetic construct; identical to SEQ ID NO: 51

SEQ ID NO: 144 - protein, 43 aa, synthetic construct; identical to SEQ ID NO: 52

SEQ ID NO: 145 - protein, 11 aa, synthetic construct
PPAALAPKRRR

SEQ ID NO: 146 - protein, 44 aa, synthetic construct; identical to SEQ ID NO: 54

SEQ ID NO: 147 - protein, 47 aa, synthetic construct
NDLDAYEREAEKLEKKNEVLRNRLAALENELATLRQEVASMKQELQS

SEQ ID NO: 148 - protein, 42 aa, synthetic construct
NEKEELKSKKAELRNRIEQLKQKREQLKQKIANLRKEIEAYK

SEQ ID NO: 149 - protein, 41 aa, synthetic construct
SIAATLENDLARLENENARLEKDIANLERDLAKLEREEAYF

SEQ ID NO: 150 - protein, 108 aa, synthetic construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 151 - protein, 292 aa, synthetic construct
LEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFM

SEQ ID NO: 152 - protein, 165 aa, synthetic construct
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 153 - protein, 94 aa, synthetic construct
MILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 154 - protein, 804 aa, synthetic construct
MSNSYDSSSIKVLKGLDAVRKRPGMYIGDTDDGTGLHHMVFEVVDNAIDEALAGHCKEIIVTIHADNSVSVQDDGRGIPTGIHPEEGVSAAEVIMTVLHAGGKFDDNSYKVSGGLHGVGVSVVNALSQKLELVIQREGKIHRQIYEHGVPQAPLAVTGETEKTGTMVRFWPSLETFTNVTEFEYEILAKRLRELSFLNSGVSIRLRDKRDGKEDHFHYEGGIKAFVEYLNKNKTPIHPNIFYFSTEKDGIGVEVALQWNDGFQENIYCFTNNIPQRDGGTHLAGFRAAMTRTLNAYMDKEGYSKKAKVSATGDDAREGLIAVVSVKVPDPKFSSQTKDKLVSSEVKSAVEQQMNELLAEYLLENPTDAKIVVGKIIDAARAREAARRAREMTRRKGALDLAGLPGKLADCQERDPALSELYLVEGDSAGGSAKQGRNRKNQAILPLKGKILNVEKARFDKMLSSQEVATLITALGCGIGRDEYNPDKLRYHSIIIMTDADVDGSHIRTLLLTFFYRQMPEIVERGHVYIAQPPLYKVKKGKQEQYIKDDEAMDQYQISIALDGATLHTNASAPALAGEALEKLVSEYNATQKMINRMERRYPKAMLKELIYQPTLTEADLSDEQTVTRWVNALVSELNDKEQHGSQWKFDVHTNAEQNLFEPIVRVRTHGVDTDYPLDHEFITGGEYRRICTLGEKLRGLLEEDAFIERGERRQPVASFEQALDWLVKESRRGLSIQRYKGLGEMNPEQLWETTMDPESRRMLRVTVKDAIAADQLFTTLMGDAVEPRRAFIEENALKAANIDI

SEQ ID NO: 155 - protein, 187 aa, synthetic construct
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 156 - protein, 111 aa, synthetic construct
MASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 157 - protein, 183 aa, synthetic construct
MNGDETKKVESEYIKKHHRHELVESQCSSTLVKHIKAPLHLVWSIVRRFDEPQKYKPFISRCVVQGKKLEVGSVREVDLKSGLPATKSTEVLEILDDNEHILGIRIVGGDHRLKNYSSTISLHSETIDGKTGTLAIESFVVDVPEGNTKEETCFFVEALIQCNLNSLADVTERLQAESMEKKI

SEQ ID NO: 158 - protein, 161 aa, synthetic construct
METSQKYHTCGSTLVQTIDAPLSLVWSILRRFDNPQAYKQFVKTCNLSSGDGGEGSVREVTVVSGLPAEFSRERLDELDDESHVMMISIIGGDHRLVNYRSKTMAFVAADTEEKTVVVESYVVDVPEGNSEEETTSFADTIVGFNLKSLAKLSERVAHLKL

SEQ ID NO: 159 - protein, 159 aa, synthetic construct
MKTSQEQHVCGSTVVQTINAPLPLVWSILRRFDNPKTFKHFVKTCKLRSGDGGEGSVREVTVVSDLPASFSLERLDELDDESHVMVISIIGGDHRLVNYQSKTTVFVAAEEEKTVVVESYVVDVPEGNTEEETTLFADTIVGCNLRSLAKLSEKMMELT

SEQ ID NO: 160 - protein, 164 aa, synthetic construct
MESSKQKRCRSSVVETIEAPLPLVWSILRSFDKPQAYQRFVKSCTMRSGGGGGKGGEGKGSVRDVTLVSGFPADFSTERLEELDDESHVMVVSIIGGNHRLVNYKSKTKVVASPEDMAKKTVVVESYVVDVPEGTSEEDTIFFVDNIIRYNLTSLAKLTKKMMK

SEQ ID NO: 161 - protein, 221 aa, synthetic construct
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 162 - protein, 190 aa, synthetic construct
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 163 - protein, 209 aa, synthetic construct
MNLAPIHDPSSSSTTTTSSSTPYGLTKDEFSTLDSIIRTHHTFPRSPNTCTSLIAHRVDAPAHAIWRFVRDFANPNKYKHFIKSCTIRVNGNGIKEIKVGTIREVSVVSGLPASTSVEILEVLDEEKRILSFRVLGGEHRLNNYRSVTSVNEFVVLEKDKKKRVYSVVLESYIVDIPQGNTEEDTRMFVDTVVKSNLQNLAVISTASPT

SEQ ID NO: 164 - protein, 207 aa, synthetic construct
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 165 - protein, 203 aa, synthetic construct
MRSPVQLQHGSDATNGFHTLQPHDQTDGPIKRVCLTRGMHVPEHVAMHHTHDVGPDQCCSSVVQMIHAPPESVWALVRRFDNPKVYKNFIRQCRIVQGDGLHVGDLREVMVVSGLPAVSSTERLEILDEERHVISFSVVGGDHRLKNYRSVTTLHASDDEGTVVVESYIVDVPPGNTEEETLSFVDTIVRCNLQSLARSTNRQ

SEQ ID NO: 166 - protein, 215 aa, synthetic construct
MPTSIQFQRSSTAAEAANATVRNYPHHHQKQVQKVSLTRGMADVPEHVELSHTHVVGPSQCFSVVVQDVEAPVSTVWSILSRFEHPQAYKHFVKSCHVVIGDGREVGSVREVRVVSGLPAAFSLERLEIMDDDRHVISFSVVGGDHRLMNYKSVTTVHESEEDSDGKKRTRVVESYVVDVPAGNDKEETCSFADTIVRCNLQSLAKLAENTSKFS

SEQ ID NO: 167 - protein, 211 aa, synthetic construct
MEMIGGDDTDTEMYGALVTAQSLRLRHLHHCRENQCTSVLVKYIQAPVHLVWSLVRRFDQPQKYKPFISRCTVNGDPEIGCLREVNVKSGLPATTSTERLEQLDDEEHILGINIIGGDHRLKNYSSILTVHPEMIDGRSGTMVMESFVVDVPQGNTKDDTCYFVESLIKCNLKSLACVSERLAAQDITNSIATFCNASNGYREKNHTETNL

SEQ ID NO: 168 - protein, 188 aa, synthetic construct
MEANGIENLTNPNQEREFIRRHHKHELVDNQCSSTLVKHINAPVHIVWSLVRRFDQPQKYKPFISRCVVKGNMEIGTVREVDVKSGLPATRSTERLELLDDNEHILSIRIVGGDHRLKNYSSIISLHPETIEGRIGTLVIESFVVDVPEGNTKDETCYFVEALIKCNLKSLADISERLAVQDTTESRV

SEQ ID NO: 169 - protein, 187 aa, synthetic construct
MMDGVEGGTAMYGGLETVQYVRTHHQHLCRENQCTSALVKHIKAPLHLVWSLVRRFDQPQKYKPFVSRCTVIGDPEIGSLREVNVKSGLPATTSTERLELLDDEEHILGIKIIGGDHRLKNYSSILTVHPEIIEGRAGTMVIESFVVDVPQGNTKDETCYFVEALIRCNLKSLADVSERLASQDITQ

SEQ ID NO: 170 - protein, 191 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 171 - protein, 432 aa, synthetic construct
MEEVSPAIAGPFRPFSETQMDFTGIRLGKGYCNNQYSNQDSENGDLMVSLPETSSCSVSGSHGSESRKVLISRINSPNLNMKESAAADIVVVDISAGDEINGSDITSEKKMISRTESRSLFEFKSVPLYGFTSICGRRPEMEDAVSTIPRFLQSSSGSMLDGRFDPQSAAHFFGVYDGHGGSQVANYCRERMHLALAEEIAKEMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKPRRKLKSKPLN

SEQ ID NO: 172 - protein, 423 aa, synthetic construct
MDEVSPAVAVPFRPFTDPHAGLRGYCNGESRVTLPESSCSGDGAMKDSSFEINTRQDSLTSSSSAMAGVDISAGDEINGSDEFDPRSMNQSEKKVLSRTESRSLFEFKCVPLYGVTSICGRRPEMEDSVSTIPRFLQVSSSSLLDGRVTNGFNPHLSAHFFGVYDGHGGSQVANYCRERMHLALTEEIVKEKPEFCDGDTWQEKWKKALFNSFMRVDSEIETVAHAPETVGSTSVVAVVFPTHIFVANCGDSRAVLCRGKTPLALSVDHKPDRDDEAARIEAAGGKVIRWNGARVFGVLAMSRSIGDRYLKPSVIPDPEVTSVRRVKEDDCLILASDGLWDVMTNEEVCDLARKRILLWHKKNAMAGEALLPAEKRGEGKDPAAMSAAEYLSKMALQKGSKDNISVVVVDLKGIRKFKSKSLN

SEQ ID NO: 173 - protein, 612 aa, synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCK

SEQ ID NO: 174 - protein, 335 aa, synthetic construct
MNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSDPLSCFNNGEAPSMWDSHVQNLYGNLGV

SEQ ID NO: 175 - protein, 533 aa, synthetic construct
MKRDHHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 176 - protein, 358 aa, synthetic construct
MAGGNEVNLNECKRIVPLNTWVLISNFKLAYKVLRRPDGSFNRDLAEFLDRKVPANSFPLDGVFSFDHVDSTTNLLTRIYQPASLLHQTRHGTLELTKPLSTTEIVPVLIFFHGGSFTHSSANSAIYDTFCRRLVTICGVVVVSVDYRRSPEHRYPCAYDDGWNALNWVKSRVWLQSGKDSNVYVYLAGDSSGGNIAHNVAVRATNEGVKVLGNILLHPMFGGQERTQSEKTLDGKYFVTIQDRDWYWRAYLPEGEDRDHPACNPFGPRGQSLKGVNFPKSLVVVAGLDLVQDWQLAYVDGLKKTGLEVNLLYLKQATIGFYFLPNNDHFHCLMEELNKFVHSIEDSQSKSSPVLLTP

SEQ ID NO: 177 - protein, 358 aa, synthetic construct; identical to SEQ ID NO: 176

SEQ ID NO: 178 - protein, 344 aa, synthetic construct
MAGSEEVNLIESKTVVPLNTWVLISNFKLAYNLLRRPDGTFNRHLAEFLDRKVPANANPVNGVFSFDVIIDRQTNLLSRVYRPADAGTSPSITDLQNPVDGEIVPVIVFFHGGSFAHSSANSAIYDTLCRRLVGLCGAVVVSVNYRRAPENRYPCAYDDGWAVLKWVNSSSWLRSKKDSKVRIFLAGDSSGGNIVHNVAVRAVESRIDVLGNILLNPMFGGTERTESEKRLDGKYFVTVRDRDWYWRAFLPEGEDREHPACSPFGPRSKSLEGLSFPKSLVVVAGLDLIQDWQLKYAEGLKKAGQEVKLLYLEQATIGFYLLPNNNHFHTVMDEIAAFVNAECQ

SEQ ID NO: 179 - protein, 11 aa, synthetic construct
YGRKKRRQRRR

SEQ ID NO: 180 - protein, 9 aa, synthetic construct
RKKRRQRRR

SEQ ID NO: 181 - protein, 8 aa, synthetic construct
RKKRRQRR

SEQ ID NO: 182 - protein, 11 aa, synthetic construct
YARAAARQARA

SEQ ID NO: 183 - protein, 11 aa, synthetic construct
THRLPRRRRRR

SEQ ID NO: 184 - protein, 11 aa, synthetic construct
GGRRARRRRRR

SEQ ID NO: 185 - DNA, 40 bp, synthetic construct
  source 1..40: mol_type = other DNA
gctcttccgatctnnnnnaccctgaagttcatctgcacca

SEQ ID NO: 186 - DNA, 40 bp, synthetic construct
  source 1..40: mol_type = other DNA
gctcttccgatctnnnnnttgaagaagatggtgcgctcct

SEQ ID NO: 187 - DNA, 48 bp, synthetic construct
  source 1..48: mol_type = other DNA
gctcttccgatctnnnnnccctccttttctattttctcttccctgcag

SEQ ID NO: 188 - DNA, 41 bp, synthetic construct
  source 1..41: mol_type = other DNA
gctcttccgatctnnnnnccgacatgtaggaccttgtgccc

SEQ ID NO: 189 - protein, 12 aa, synthetic construct
RRQRRTSKLMKR

SEQ ID NO: 190 - protein, 27 aa, synthetic construct
GWTLNSAGYLLGKINLKALAALAKKIL

SEQ ID NO: 191 - protein, 33 aa, synthetic construct
KALAWEAKLAKALAKALAKHLAKALAKALKCEA

SEQ ID NO: 192 - protein, 16 aa, synthetic construct
RQIKIWFQNRRMKWKK

SEQ ID NO: 193 - protein, 18 aa, synthetic construct
WPTLVTTLTHGVQCFSRY

SEQ ID NO: 194 - DNA, 54 bp, synthetic construct
  source 1..54: mol_type = other DNA
tggcccaccctcgtgaccaccctgacccatggcgtgcagtgcttcagccgctac

SEQ ID NO: 195 - DNA, 23 bp, synthetic construct
  source 1..23: mol_type = other DNA
gggagcactggtgggactgggta

SEQ ID NO: 196 - protein, 18 aa, synthetic construct
WPTLVTTLTYGVQCFSRY

SEQ ID NO: 197 - DNA, 54 bp, synthetic construct
  source 1..54: mol_type = other DNA
tggcccaccctcgtgaccaccctgacctatggcgtgcagtgcttcagccgctac

SEQ ID NO: 198 - protein, 14 aa, synthetic construct
DVKPSNILVNSRGE

SEQ ID NO: 199 - DNA, 49 bp, synthetic construct
  source 1..49: mol_type = other DNA
ccctgcagatgtcaagccctccaacatcctagtcaactcccgtggggag

SEQ ID NO: 200 - protein, 16 aa, synthetic construct
IKLCDFGVSGQLIDSM

SEQ ID NO: 201 - DNA, 49 bp, synthetic construct
  source 1..49: mol_type = other DNA
atcaagctctgtgactttggggtcagcgggcagctcatcgactccatgg

SEQ ID NO: 202 - protein, 18 aa, synthetic construct
WPTLVTTLTHGVQCFSRY

SEQ ID NO: 203 - DNA, 54 bp, synthetic construct; identical to SEQ ID NO: 194
  source 1..54: mol_type = other DNA

SEQ ID NO: 204 - DNA, 23 bp, synthetic construct
  source 1..23: mol_type = other DNA
gggagcactggtgggactgggta

SEQ ID NO: 205 - DNA, 21 bp, synthetic construct
  source 1..21: mol_type = other DNA
gggagcactggtgggactggg

SEQ ID NO: 206 - protein, 18 aa, synthetic construct
WPTLVTTLTHGVQCFSRY

SEQ ID NO: 207 - DNA, 54 bp, synthetic construct; identical to SEQ ID NO: 194
  source 1..54: mol_type = other DNA

SEQ ID NO: 208 - DNA, 25 bp, synthetic construct
  source 1..25: mol_type = other DNA
cgggtgggagcactggtgggactgg

SEQ ID NO: 209 - DNA, 25 bp, synthetic construct
  source 1..25: mol_type = other DNA
gggtgggagcactggtgggactggg

SEQ ID NO: 210 - DNA, 26 bp, synthetic construct
  source 1..26: mol_type = other DNA
ggtgggagcactggtgggactgggtg

SEQ ID NO: 211 - DNA, 26 bp, synthetic construct
  source 1..26: mol_type = other DNA
tgggagcactggtgggactgggtacg

SEQ ID NO: 212 - DNA, 26 bp, synthetic construct
  source 1..26: mol_type = other DNA
gggagcactggtgggactgggtaccg

SEQ ID NO: 213 - DNA, 25 bp, synthetic construct
  source 1..25: mol_type = other DNA
ggagcactggtgggactgggtaccg

SEQ ID NO: 214 - DNA, 26 bp, synthetic construct
  source 1..26: mol_type = other DNA
agcactggtgggactgggtaccgcag

SEQ ID NO: 215 - DNA, 26 bp, synthetic construct
  source 1..26: mol_type = other DNA
tggtgggactgggtaccgcacgtcag

SEQ ID NO: 216 - DNA, 26 bp, synthetic construct
  source 1..26: mol_type = other DNA
ggtgggactgggtaccgcacgtcacg

SEQ ID NO: 217 - DNA, 26 bp, synthetic construct
  source 1..26: mol_type = other DNA
tgggactgggtaccgcacgtcacgag

SEQ ID NO: 218 - DNA, 26 bp, synthetic construct
  source 1..26: mol_type = other DNA
gggactgggtaccgcacgtcacgaag

SEQ ID NO: 219 - DNA, 25 bp, synthetic construct
  source 1..25: mol_type = other DNA
ggactgggtaccgcacgtcacgaag

SEQ ID NO: 220 - DNA, 25 bp, synthetic construct
  source 1..25: mol_type = other DNA
tgggtaccgcacgtcacgaagtcgg

SEQ ID NO: 221 - DNA, 26 bp, synthetic construct
  source 1..26: mol_type = other DNA
gggtaccgcacgtcacgaagtcggcg

SEQ ID NO: 222 - DNA, 25 bp, synthetic construct
  source 1..25: mol_type = other DNA
ggtaccgcacgtcacgaagtcggcg

SEQ ID NO: 223 - DNA, 25 bp, synthetic construct
  source 1..25: mol_type = other DNA
cgcacgtcacgaagtcggcgatggg

SEQ ID NO: 224 - DNA, 26 bp, synthetic construct
  source 1..26: mol_type = other DNA
gccatggcgtgcagtgcttcagccgc

SEQ ID NO: 225 - DNA, 25 bp, synthetic construct
  source 1..25: mol_type = other DNA
gacccatggcgtgcagtgcttcagc

SEQ ID NO: 226 - DNA, 26 bp, synthetic construct
  source 1..26: mol_type = other DNA
gcaccctgacccatggcgtgcagtgc

SEQ ID NO: 227 - DNA, 26 bp, synthetic construct
  source 1..26: mol_type = other DNA
gaccaccctgacccatggcgtgcagt

SEQ ID NO: 228 - DNA, 25 bp, synthetic construct
  source 1..25: mol_type = other DNA
gtgaccaccctgacccatggcgtgc

SEQ ID NO: 229 - DNA, 26 bp, synthetic construct
  source 1..26: mol_type = other DNA
gtcgtgaccaccctgacccatggcgt

SEQ ID NO: 230 - DNA, 26 bp, synthetic construct
  source 1..26: mol_type = other DNA
gcctcgtgaccaccctgacccatggc

SEQ ID NO: 231 - DNA, 26 bp, synthetic construct
  source 1..26: mol_type = other DNA
gccctcgtgaccaccctgacccatgg

SEQ ID NO: 232 - DNA, 40 bp, synthetic construct
  source 1..40: mol_type = other DNA
agctctgtgactttggggtcagcgggcagctcatcgactc

SEQ ID NO: 233 - DNA, 40 bp, synthetic construct; identical to SEQ ID NO: 232
  source 1..40: mol_type = other DNA

SEQ ID NO: 234 - DNA, 40 bp, synthetic construct
  source 1..40: mol_type = other DNA
agctctgtgactttggggtcagctgcccgctcatcgactc

SEQ ID NO: 235 - DNA, 40 bp, synthetic construct
  source 1..40: mol_type = other DNA
agctctgtgactttgggcacagcgggcagctcatcgactc

SEQ ID NO: 236 - DNA, 40 bp, synthetic construct; identical to SEQ ID NO: 232
  source 1..40: mol_type = other DNA

SEQ ID NO: 237 - DNA, 40 bp, synthetic construct; identical to SEQ ID NO: 232
  source 1..40: mol_type = other DNA

SEQ ID NO: 238 - DNA, 40 bp, synthetic construct; identical to SEQ ID NO: 234
  source 1..40: mol_type = other DNA

SEQ ID NO: 239 - protein, 18 aa, synthetic construct
WPTLVTTLTHGVQCFSRY

SEQ ID NO: 240 - DNA, 54 bp, synthetic construct; identical to SEQ ID NO: 194
  source 1..54: mol_type = other DNA

SEQ ID NO: 241 - protein, 18 aa, synthetic construct
WPTLVTTLTHGVQCFSRY

SEQ ID NO: 242 - DNA, 54 bp, synthetic construct; identical to SEQ ID NO: 194
  source 1..54: mol_type = other DNA

SEQ ID NO: 243 - protein, 18 aa, synthetic construct
WPTLVTTLTHGVQCFSRY

SEQ ID NO: 244 - DNA, 54 bp, synthetic construct; identical to SEQ ID NO: 194
  source 1..54: mol_type = other DNA

SEQ ID NO: 245 - DNA, 18 bp, synthetic construct
  source 1..18: mol_type = other DNA
gggagcactggtgggact

SEQ ID NO: 246 - DNA, 19 bp, synthetic construct
  source 1..19: mol_type = other DNA
gggagcactggtgggactg

SEQ ID NO: 247 - DNA, 20 bp, synthetic construct
  source 1..20: mol_type = other DNA
gggagcactggtgggactgg

SEQ ID NO: 248 - DNA, 21 bp, synthetic construct
  source 1..21: mol_type = other DNA
gggagcactggtgggactggg

SEQ ID NO: 249 - DNA, 18 bp, synthetic construct
  source 1..18: mol_type = other DNA
ggagcactggtgggactg

SEQ ID NO: 250 - DNA, 19 bp, synthetic construct
  source 1..19: mol_type = other DNA
ggagcactggtgggactgg

SEQ ID NO: 251 - DNA, 20 bp, synthetic construct
  source 1..20: mol_type = other DNA
ggagcactggtgggactggg

SEQ ID NO: 252 - DNA, 21 bp, synthetic construct
  source 1..21: mol_type = other DNA
ggagcactggtgggactgggt

SEQ ID NO: 253 - DNA, 22 bp, synthetic construct
  source 1..22: mol_type = other DNA
ggagcactggtgggactgggtg

SEQ ID NO: 254 - DNA, 18 bp, synthetic construct
  source 1..18: mol_type = other DNA
gcactggtgggactgggt

SEQ ID NO: 255 - DNA, 19 bp, synthetic construct
  source 1..19: mol_type = other DNA
gcactggtgggactgggtg

SEQ ID NO: 256 - DNA, 19 bp, synthetic construct
  source 1..19: mol_type = other DNA
gcactggtgggactgggta

SEQ ID NO: 257 - DNA, 20 bp, synthetic construct
  source 1..20: mol_type = other DNA
gcactggtgggactgggtac

SEQ ID NO: 258 - DNA, 21 bp, synthetic construct
  source 1..21: mol_type = other DNA
gcactggtgggactgggtacg

SEQ ID NO: 259 - DNA, 21 bp, synthetic construct
  source 1..21: mol_type = other DNA
gcactggtgggactgggtacc

SEQ ID NO: 260 - DNA, 22 bp, synthetic construct
  source 1..22: mol_type = other DNA
gcactggtgggactgggtaccg

SEQ ID NO: 261 - DNA, 18 bp, synthetic construct
  source 1..18: mol_type = other DNA
ggactgggtaccgcacgt

SEQ ID NO: 262 - DNA, 19 bp, synthetic construct
  source 1..19: mol_type = other DNA
ggactgggtaccgcacgtg

SEQ ID NO: 263 - DNA, 19 bp, synthetic construct
  source 1..19: mol_type = other DNA
ggactgggtaccgcacgtc

SEQ ID NO: 264 - DNA, 20 bp, synthetic construct
  source 1..20: mol_type = other DNA
ggactgggtaccgcacgtcg

SEQ ID NO: 265 - DNA, 20 bp, synthetic construct
  source 1..20: mol_type = other DNA
ggactgggtaccgcacgtca

SEQ ID NO: 266 - DNA, 21 bp, synthetic construct
  source 1..21: mol_type = other DNA
ggactgggtaccgcacgtcac

SEQ ID NO: 267 - DNA, 22 bp, synthetic construct
  source 1..22: mol_type = other DNA
ggactgggtaccgcacgtcacg

SEQ ID NO: 268 - DNA, 18 bp, synthetic construct
  source 1..18: mol_type = other DNA
ctgggtaccgcacgtcac

SEQ ID NO: 269 - DNA, 19 bp, synthetic construct
  source 1..19: mol_type = other DNA
ctgggtaccgcacgtcacg

SEQ ID NO: 270 - DNA, 20 bp, synthetic construct
  source 1..20: mol_type = other DNA
ctgggtaccgcacgtcacga

SEQ ID NO: 271 - DNA, 21 bp, synthetic construct
  source 1..21: mol_type = other DNA
ctgggtaccgcacgtcacgaa

SEQ ID NO: 272 - DNA, 18 bp, synthetic construct
  source 1..18: mol_type = other DNA
tgggtaccgcacgtcacg

SEQ ID NO: 273 - DNA, 19 bp, synthetic construct
  source 1..19: mol_type = other DNA
tgggtaccgcacgtcacga

SEQ ID NO: 274 - DNA, 20 bp, synthetic construct
  source 1..20: mol_type = other DNA
tgggtaccgcacgtcacgaa

SEQ ID NO: 275 - DNA, 21 bp, synthetic construct
  source 1..21: mol_type = other DNA
tgggtaccgcacgtcacgaag

SEQ ID NO: 276 - DNA, 18 bp, synthetic construct
  source 1..18: mol_type = other DNA
accgcacgtcacgaagtc

SEQ ID NO: 277 - DNA, 19 bp, synthetic construct
  source 1..19: mol_type = other DNA
accgcacgtcacgaagtcg

SEQ ID NO: 278 - DNA, 20 bp, synthetic construct
  source 1..20: mol_type = other DNA
accgcacgtcacgaagtcgg

SEQ ID NO: 279 - DNA, 21 bp, synthetic construct
  source 1..21: mol_type = other DNA
accgcacgtcacgaagtcggc

SEQ ID NO: 280 - DNA, 22 bp, synthetic construct
  source 1..22: mol_type = other DNA
accgcacgtcacgaagtcggcg

SEQ ID NO: 281 - DNA, 18 bp, synthetic construct
  source 1..18: mol_type = other DNA
ccgcacgtcacgaagtcg

SEQ ID NO: 282 - DNA, 19 bp, synthetic construct
  source 1..19: mol_type = other DNA
ccgcacgtcacgaagtcgg

SEQ ID NO: 283 - DNA, 20 bp, synthetic construct
  source 1..20: mol_type = other DNA
ccgcacgtcacgaagtcggc

SEQ ID NO: 284 - DNA, 21 bp, synthetic construct
  source 1..21: mol_type = other DNA
ccgcacgtcacgaagtcggcg

SEQ ID NO: 285 - protein, 18 aa, synthetic construct
WPTLVTTLTHGVQCFSRV

SEQ ID NO: 286 - DNA, 54 bp, synthetic construct; identical to SEQ ID NO: 194
  source 1..54: mol_type = other DNA

SEQ ID NO: 287 - DNA, 21 bp, synthetic construct
  source 1..21: mol_type = other DNA
gggagcactggtgggactggg

SEQ ID NO: 288 - DNA, 21 bp, synthetic construct
  source 1..21: mol_type = other DNA
gggagcactggtgggactgga

SEQ ID NO: 289 - DNA, 21 bp, synthetic construct
  source 1..21: mol_type = other DNA
gcactggtgggactgggtacc

SEQ ID NO: 290 - DNA, 21 bp, synthetic construct
  source 1..21: mol_type = other DNA
gcactggtgggactgggtacg

SEQ ID NO: 291 - DNA, 21 bp, synthetic construct
  source 1..21: mol_type = other DNA
ggactgggtaccgcacgtcac

SEQ ID NO: 292 - DNA, 21 bp, synthetic construct
  source 1..21: mol_type = other DNA
ggactgggtaccgcacgtcag

SEQ ID NO: 293 - DNA, 21 bp, synthetic construct
  source 1..21: mol_type = other DNA
tgggtaccgcacgtcacgaag

SEQ ID NO: 294 - DNA, 21 bp, synthetic construct
  source 1..21: mol_type = other DNA
tgggtaccgcacgtcacgaaa

SEQ ID NO: 295 - protein, 15 aa, synthetic construct
TLVTTLTHGVQCFSR

SEQ ID NO: 296 - DNA, 45 bp, synthetic construct
  source 1..45: mol_type = other DNA
accctcgtgaccaccctgacccatggcgtgcagtgcttcagccgc

SEQ ID NO: 297 - protein, 15 aa, synthetic construct
TLVTTLTYGVQCFSR

SEQ ID NO: 298 - DNA, 45 bp, synthetic construct
  source 1..45: mol_type = other DNA
accctcgtgaccaccctgacctatggcgtgcagtgcttcagccgc